(19) United States
(12) Patent Application Publication
(10) Pub. No.: US 2012/0309746 A1
(43) Pub. Date: Dec. 6, 2012

(54) TRIAZOLOPYRIDINE COMPOUNDS

(76) Inventors: Johannes Cornelius Hermann, Jersey City, NJ (US); Matthew C. Lucas, Verona, NJ (US); Kin-Chun Thomas Luk, North Caldwell, NJ (US); Fernando Padilla, Verona, NJ (US); Jutta Wanner, Montclair, NJ (US); Wenwei Xie, Beijing (CN); Xiaohu Zhang, Beijing (CN)

(21) Appl. No.: 13/479,730

(22) Filed: May 24, 2012

Related U.S. Application Data
(63) Continuation of application No. PCT/CN2011/074985, filed on May 31, 2011.

(51) Int. Cl. C07D 471/04; A61K 31/551; A61P 37/00; A61P 9/00; A61P 3/10; A61P 37/06; A61P 19/02; A61P 35/04; A61P 1/04; A61P 17/00; A61P 25/28; A61P 17/06; A61K 31/437; A61P 35/02

(52) U.S. Cl. 514/218; 546/119; 514/303; 540/597

(57) ABSTRACT

The present invention relates to the use of novel triazolopyridine derivatives of formula I:

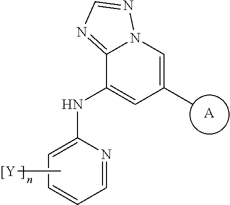

wherein all variable substituents are defined as described herein, which are SYK inhibitors and are useful for the treatment of auto-immune and inflammatory diseases.

### CROSS-REFERENCE TO RELATED APPLICATIONS

This application is entitled to the benefit of PCT patent application PCT/CN2011/074985, filed May 31, 2011, the disclosure of which is incorporated herein by reference.

### FIELD OF THE INVENTION

Protein kinases constitute one of the largest families of human enzymes and regulate many different signaling processes by adding phosphate groups to proteins; particularly tyrosine kinases phosphorylate proteins on the alcohol moiety of tyrosine residues. The tyrosine kinase family includes members that control cell growth, migration, and differentiation. Abnormal kinase activity has been implicated in a variety of human diseases including cancers, autoimmune and inflammatory diseases. Since protein kinases are among the key regulators of cell signaling they provide a means to modulate cellular function with small molecule inhibitors of kinase activity and thus make good drug design targets. In addition to treatment of kinase-mediated disease processes, selective and efficacious inhibitors of kinase activity are also useful for investigation of cell signaling processes and identification of other cellular targets of therapeutic interest.

SYK (Spleen Tyrosine Kinase) is a non-receptor tyrosine kinase that is essential for B-cell activation through BCR signaling. SYK becomes activated upon binding to phosphoryated BCR and thus initiates the early signaling events following BCR activation. Mice deficient in SYK exhibit an early block in B-cell development. Therefore inhibition of SYK enzymatic activity in cells is proposed as a treatment for autoimmune disease through its effects on autoantibody production.

In addition to the role of SYK in BCR signaling and B-cell activation, it also plays a key role in Fc RI mediated mast cell degranulation and eosinophil activation. Thus, SYK is implicated in allergic disorders including asthma. SYK binds to the phosphorylated gamma chain of FcγRI via its SH2 domains and is essential for downstream signaling. SYK deficient mast cells demonstrate defective degranulation, arachidonic acid and cytokine secretion. This also has been shown for pharmacologic agents that inhibit SYK activity in mast cells. Treatment with SYK antisense oligonucleotides inhibits antigen-induced infiltration of eosinophils and neutrophils in an animal model of asthma. SYK deficient eosinophils also show impaired activation in response to Fc R stimulation. Therefore, small molecule inhibitors of SYK will be useful for treatment of allergy-induced inflammatory diseases including asthma.

In view of the numerous conditions that are contemplated to benefit by treatment involving modulation of the SYK pathway it is immediately apparent that new compounds that modulate the SYK pathway and methods of using these compounds should provide substantial therapeutic benefits to a wide variety of patients. Provided herein are novel compounds for use in the therapeutic treatment of auto-immune and inflammatory diseases by targeting the SYK pathway or by inhibition of SYK kinase.

### SUMMARY OF THE INVENTION

The application provides a compound of Formula I

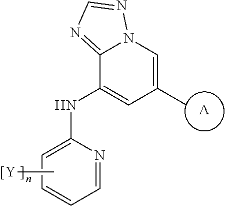

wherein:
A is phenyl or heterocycloalkyl, optionally substituted with one or more A′;
each A′ is independently C(═O)NHR, C(═O)R, C(═O)OR, OR, NHC(═O)R, CH₂NHR, lower alkyl, hydroxy lower alkyl, or hydroxy lower alkyl amino;
   each R is independently H, or R′;
      each R′ is independently lower alkyl, heterocycloalkyl, phenyl, heteroaryl, heteroaryl lower alkyl, or bicyclic heteroaryl, optionally substituted with one or more R″;
         each R″ is independently hydroxy, lower alkyl amido, carboxy, oxo, lower alkoxy, lower alkyl amino, or lower dialkyl amino;
            each Y is independently lower alkyl, lower alkoxy, lower haloalkyl, lower hydroxyalkyl, or heterocycloalkyl, optionally substituted with lower alkyl; and
            n is 0, 1, or 2;
            or a pharmaceutically acceptable salt thereof.

The application provides a method for treating an inflammatory or autoimmune condition comprising administering to a patient in need thereof a therapeutically effective amount of the compound of Formula I.

The application provides a pharmaceutical composition comprising the compound of Formula I, admixed with at least one pharmaceutically acceptable carrier, excipient or diluent.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “ [symbol] ” drawn through a bond each refer to the point of attachment of a functional group or other chemical moiety to the rest of the molecule of which it is a part. Thus, for example:

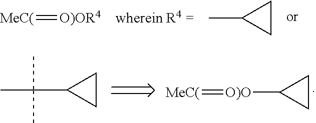

A bond drawn into ring system (as opposed to connected at a distinct vertex) indicates that the bond may be attached to any of the suitable ring atoms

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “spirocycloalkyl”, as used herein, means a spirocyclic cycloalkyl group, such as, for example, spiro[3.3]heptane. The term spiroheterocycloalkyl, as used herein, means a spirocyclic heterocycloalkyl, such as, for example, 2,6-diaza spiro[3.3]heptane.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “ester” as used herein denotes a group of formula —C(═O)OR wherein R is lower alkyl as defined herein.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ)or a branched saturated divalent hydrocarbon radical of 2 to 10 carbon atoms (e.g., —CHMe— or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “PCy₃” refers to a phosphine trisubstituted with three cyclic moieties.

The terms “haloalkoxy” or “halo-lower alkoxy” or “lower haloalkoxy” refers to a lower alkoxy group, wherein one or more carbon atoms are substituted with one or more halogen atoms.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to a cycloalkyl composed of 3 to 7 carbons in the carbocyclic ring.

The term “carboxy-alkyl” as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic or partially unsaturated ring containing four to eight atoms per ring, incorporating one or more N, O, or S heteroatoms, the remaining ring atoms being carbon, with the understanding that the attachment point of the heteroaryl radical will be on an aromatic or partially unsaturated ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, oxazinyl, pyrrolyl, pyrazolyl, imidazolyl, oxazolyl, 4,5-Dihydro-oxazolyl, 5,6-Dihydro-4H-[1,3]oxazolyl, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole, naphthyridinyl, 5,6,7,8-Tetrahydro-[1,6]naphthyridinyl, and benzisothiazole. Bicyclic moieties can be optionally substituted on either ring, however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, and ionic forms thereof, unless otherwise indicated. Examples of heterocyclic radicals include, but are not limited to, morpholinyl, piperazinyl, piperidinyl, azetidinyl, pyrrolidinyl, hexahydroazepinyl, oxetanyl, tetrahydrofuranyl, tetrahydrothiophenyl, oxazolidinyl, thiazolidinyl, isoxazolidinyl, tetrahydropyranyl, thiomorpholinyl, quinuclidinyl and imidazolinyl, and ionic forms thereof. Examples may also be bicyclic, such as, for example, 3,8-diaza-bicyclo[3.2.1]octane, 2,5-diaza-bicyclo[2.2.2]octane, or octahydro-pyrazino[2,1-c][1,4]oxazine.

### Inhibitors of SYK

The application provides a compound of Formula I

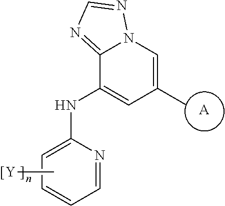

wherein:
A is phenyl or heterocycloalkyl, optionally substituted with one or more A′;
each A′ is independently C(═O)NHR, C(═O)R, C(═O)OR, OR, NHC(═O)R, CH₂NHR, lower alkyl, hydroxy lower alkyl, or hydroxy lower alkyl amino;
   each R is independently H, or R′;
      each R′ is independently lower alkyl, heterocycloalkyl, phenyl, heteroaryl, heteroaryl lower alkyl, or bicyclic heteroaryl, optionally substituted with one or more R″;
         each R″ is independently hydroxy, lower alkyl amido, carboxy, oxo, lower alkoxy, lower alkyl amino, or lower dialkyl amino;
            each Y is independently lower alkyl, lower alkoxy, lower haloalkyl, lower hydroxyalkyl, or heterocycloalkyl, optionally substituted with lower alkyl; and
            n is 0, 1, or 2;
            or a pharmaceutically acceptable salt thereof.

The application provides formula I, wherein A is phenyl.

The application provides formula I, wherein A′ is C(═O)NHR.

The application provides formula I, wherein A is phenyl and A′ is C(═O)NHR.

The application provides formula I, wherein R is H.

The application provides formula I, wherein R is H and A is phenyl.

The application provides formula I, wherein R is H and A′ is C(═O)NHR.

The application provides formula I, wherein R is H, A is phenyl, and A′ is C(═O)NHR.

The application provides formula I, wherein R is phenyl.

The application provides formula I, wherein R is phenyl and A is phenyl.

The application provides formula I, wherein R is phenyl and A′ is C(═O)NHR.

The application provides formula I, wherein R is phenyl, A is phenyl, and A′ is C(═O)NHR.

The application provides formula I, wherein R is phenyl optionally substituted with carboxy.

The application provides formula I, wherein R is phenyl optionally substituted with carboxy, and A is phenyl.

The application provides formula I, wherein R is phenyl optionally substituted with carboxy, and A′ is C(═O)NHR.

The application provides formula I, wherein R is phenyl optionally substituted with carboxy, A is phenyl, and A′ is C(═O)NHR.

The application provides formula I, wherein R is phenyl optionally substituted with carboxy and methoxy.

The application provides formula I, wherein R is phenyl optionally substituted with carboxy and methoxy, and A is phenyl.

The application provides formula I, wherein R is phenyl optionally substituted with carboxy and methoxy, and A′ is C(═O)NHR.

The application provides formula I, wherein R is phenyl optionally substituted with carboxy and methoxy, A is phenyl, and A′ is C(═O)NHR.

The application provides formula I, wherein R is heteroaryl, optionally substituted with one or more R″.

The application provides formula I, wherein R is heteroaryl, optionally substituted with one or more R″ and A is phenyl.

The application provides formula I, wherein R is heteroaryl, optionally substituted with one or more R″ and A′ is C(═O)NHR.

The application provides formula I, wherein R is heteroaryl, optionally substituted with one or more R″, A is phenyl, and A′ is C(═O)NHR.

The application provides formula I, wherein R is bicyclic heteroaryl, optionally substituted with one or more R″.

The application provides formula I, wherein R is bicyclic heteroaryl optionally substituted with one or more R″ and A is phenyl.

The application provides formula I, wherein R is optionally substituted with one or more R″ and A′ is C(═O)NHR.

The application provides formula I, wherein R is optionally substituted with one or more R″, A is phenyl, and A′ is C(═O)NHR.

The application provides formula I, wherein n is 2 and both Y are methoxy.

The application provides formula I, wherein n is 1 and Y is methyl pyrrolidinyl.

The application provides a compound selected from the group consisting of:
[1,4]Diazepan-1-yl-{3-[8-(5,6-dimethoxy-pyridin-2-ylamino)-[1,2,4]triazolo[1,5-a]pyridin-6-yl]-phenyl}-methanone;
3-[8-(5,6-Dimethoxy-pyridin-2-ylamino)-[1,2,4]triazolo[1,5-a]pyridin-6-yl]-N-(4-N-methyl)-benzamide
4-{3-[8-(5,6-Dimethoxy-pyridin-2-ylamino)-[1,2,4]triazolo[1,5-a]pyridin-6-yl]-benzoylamino}-benzoic acid;
4-[8-(5,6-Dimethoxy-pyridin-2-ylamino)-[1,2,4]triazolo[1,5-a]pyridin-6-yl]-N-(2-pyridin-4-yl-ethyl)-benzamide;
4-{3-[8-(5,6-Dimethoxy-pyridin-2-ylamino)-[1,2,4]triazolo[1,5-a]pyridin-6-yl]-benzoylamino}-2-methoxy-benzoic acid;
3-[8-(5,6-Dimethoxy-pyridin-2-ylamino)-[1,2,4]triazolo[1,5-a]pyridin-6-yl]-N-(2-oxo-2,3-dihydro-1H-indol-5-yl)-benzamide;
3-[8-(5,6-Dimethoxy-pyridin-2-ylamino)-[1,2,4]triazolo[1,5-a]pyridin-6-yl]-N-(1H-indazol-5-yl)-benzamide;
4-(3-{8-[6-(2-Methyl-pyrrolidin-1-yl)-pyridin-2-ylamino]-[1,2,4]triazolo[1,5-a]pyridin-6-yl}-benzoylamino)-benzoic acid;
4-{8-[6-(2-Methyl-pyrrolidin-1-yl)-pyridin-2-ylamino]-[1,2,4]triazolo[1,5-a]pyridin-6-yl}-benzoic acid methyl ester;
N-{1-[8-(5,6-Dimethoxy-pyridin-2-ylamino)-[1,2,4]triazolo[1,5-a]pyridin-6-yl]-piperidin-3-yl}-terephthalamic acid;
2-Methoxy-4-(3-{8-[6-(2-methyl-pyrrolidin-1-yl)-pyridin-2-ylamino]-[1,2,4]triazolo[1,5-a]pyridin-6-yl}-benzoylamino)-benzoic acid;
4-{8-[6-(2-Methyl-pyrrolidin-1-yl)-pyridin-2-ylamino]-[1,2,4]triazolo[1,5-a]pyridin-6-yl}-benzoic acid;
3-{8-[6-(2-Methyl-pyrrolidin-1-yl)-pyridin-2-ylamino]-[1,2,4]triazolo[1,5-a]pyridin-6-yl}-benzoic acid methyl ester;
3-{8-[6-(2-Methyl-pyrrolidin-1-yl)-pyridin-2-ylamino]-[1,2,4]triazolo[1,5-a]pyridin-6-yl}-benzoic acid;
4-({1-[8-(5,6-Dimethoxy-pyridin-2-ylamino)-[1,2,4]triazolo[1,5-a]pyridin-6-yl]-piperidine-3-carbonyl}-amino)-benzoic acid;
3-{8-[(3aS,6aS)-6-(Hexahydro-cyclopenta[b]pyrrol-1-yl)-pyridin-2-ylamino]-[1,2,4]triazolo[1,5-a]pyridin-6-yl}-benzamide;
4-[8-(5,6-Dimethoxy-pyridin-2-ylamino)-[1,2,4]triazolo[1,5-a]pyridin-6-yl]-N-(2-dimethylamino-ethyl)-benzamide;
4-[8-(5,6-Dimethoxy-pyridin-2-ylamino)-[1,2,4]triazolo[1,5-a]pyridin-6-yl]-benzamide;
4-{8-[6-((S)-2-Methyl-pyrrolidin-1-yl)-pyridin-2-ylamino]-[1,2,4]triazolo[1,5-a]pyridin-6-yl}-benzamide;
{3-[8-(5,6-Dimethoxy-pyridin-2-ylamino)-[1,2,4]triazolo[1,5-a]pyridin-6-yl]-phenyl}-methanol;
N-(2-Dimethylamino-ethyl)-4-{8-[6-((S)-2-methyl-pyrrolidin-1-yl)-pyridin-2-ylamino]-[1,2,4]triazolo[1,5-a]pyridin-6-yl}-benzamide; compound with trifluoro-acetic acid;
(3-{8-[6-((S)-2-Methyl-pyrrolidin-1-yl)-pyridin-2-ylamino]-[1,2,4]triazolo[1,5-a]pyridin-6-yl}-phenyl)-methanol;
2-(3-{8-[6-((S)-2-Methyl-pyrrolidin-1-yl)-pyridin-2-ylamino]-[1,2,4]triazolo[1,5-a]pyridin-6-yl}-benzylamino)-ethanol;
3-{8-[6-((S)-2-Methyl-pyrrolidin-1-yl)-pyridin-2-ylamino]-[1,2,4]triazolo[1,5-a]pyridin-6-yl}-benzamide; and
[6-((S)-2-Methyl-pyrrolidin-1-yl)-pyridin-2-yl]-{6-[3-(piperidin-4-ylaminomethyl)-phenyl]-[1,2,4]triazolo[1,5-a]pyridin-8-yl}-amine.

The application provides a method for treating an inflammatory or autoimmune condition comprising administering to a patient in need thereof a therapeutically effective amount of the compound of Formula I.

The application provides the above method, further comprising administering an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, or an agent for treating immunodeficiency disorders.

The application provides a method for treating an inflammatory condition comprising administering to a patient in need thereof a therapeutically effective amount of the compound of Formula I.

The application provides a method for treating rheumatoid arthritis comprising administering to a patient in need thereof a therapeutically effective amount of the compound of Formula I.

The application provides a method for treating asthma comprising administering to a patient in need thereof a therapeutically effective amount of the compound of Formula I.

The application provides a method for treating an immune disorder including lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease, and Leukemia, comprising administering to a patient in need thereof a therapeutically effective amount of the compound of Formula I.

The application provides a method for treating an inflammatory condition comprising co-administering to a patient in need thereof a therapeutically effective amount of an anti-inflammatory compound in combination with the compound of Formula I.

The application provides a method for treating an immune disorder comprising co-administering to a patient in need thereof a therapeutically effective amount of an immunosuppressant compound in combination with the compound of Formula I.

The application provides a pharmaceutical composition comprising the compound of Formula I, admixed with at least one pharmaceutically acceptable carrier, excipient or diluent.

The application provides the above pharmaceutical composition, further comprising an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, and an agent for treating immunodeficiency disorders.

The application provides the use of the compound of Formula I for the manufacture of a medicament useful for the treatment of disorders associated with Syk.

The application provides the use of the compound of Formula I for the manufacture of a medicament useful for the treatment of rheumatoid arthritis.

A compound, method, or composition as described herein.

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system or Struct=Name, a CambridgeSoft® application, for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of triazolopyridine compounds according to generic Formula I.

TABLE I

| Compound | Nomenclature | Structure |
|---|---|---|
| I-1 | [1,4]Diazepan-1-yl-{3-[8-(5,6- dimethoxy-pyridin-2-ylamino)- [1,2,4]triazolo[1,5-a]pyridin-6-yl]- phenyl}-methanone | 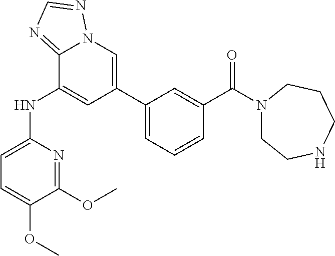 |
| I-2 | 3-[8-(5,6-Dimethoxy-pyridin-2- ylamino)-[1,2,4]triazolo [1,5-a]pyridin-6-yl]-N-(4-N- methyl)-benzamide | 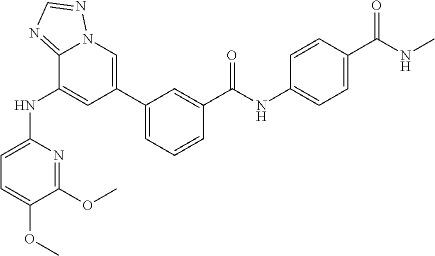 |
| I-3 | 4-{3-[8-(5,6-Dimethoxy-pyridin-2- ylamino)-[1,2,4]triazolo[1,5- a]pyridin-6-yl]-benzoylamino}- benzoic acid | 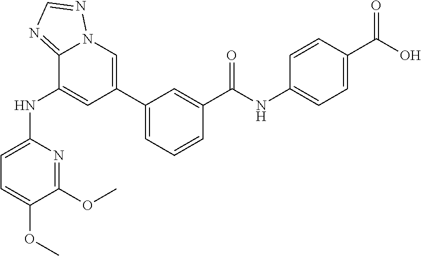 |
| I-4 | 4-[8-(5,6-Dimethoxy-pyridin-2- ylamino)-[1,2,4]triazolo[1,5- a]pyridin-6-yl]-N-(2-pyridin-4-yl- ethyl)-benzamide | 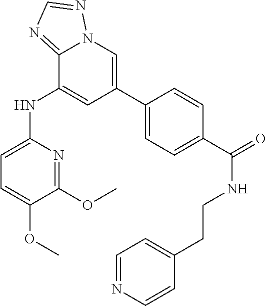 |
| I-5 | 4-{3-[8-(5,6-Dimethoxy-pyridin-2- ylamino)-[1,2,4]triazolo[1,5- a]pyridin-6-yl]-benzoylamino}-2- methoxy-benzoic acid | 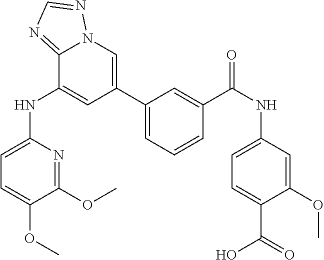 |
| I-6 | 3-[8-(5,6-Dimethoxy-pyridin-2- ylamino)-[1,2,4]triazolo[1,5- a]pyridin-6-yl]-N-(2-oxo-2,3-dihydro- 1H-indol-5-yl)-benzamide | 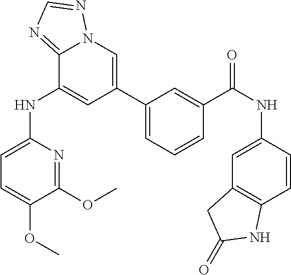 |
| I-7 | 3-[8-(5,6-Dimethoxy-pyridin-2- ylamino)-[1,2,4]triazolo[1,5- a]pyridin-6-yl]-N-(1H-indazol-5-yl)- benzamide | 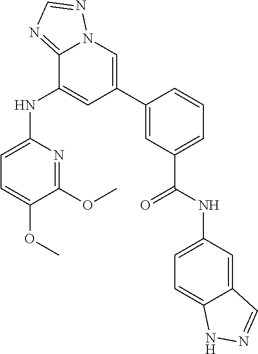 |
| I-8 | 4-(3-{8-[6-(2-Methyl-pyrrolidin-1- yl)-pyridin-2-ylamino]- [1,2,4]triazolo[1,5-a]pyridin-6-yl}- benzoylamino)-benzoic acid | 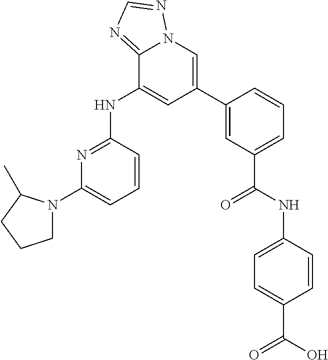 |
| I-9 | 4-{8-[6-(2-Methyl-pyrrolidin-1-yl)- pyridin-2-ylamino]- [1,2,4]triazolo[1,5-a]pyridin-6-yl}- benzoic acid methyl ester | 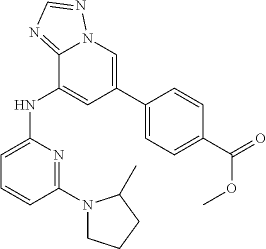 |
| I-10 | N-{1-[8-(5,6-Dimethoxy-pyridin-2- ylamino)-[1,2,4]triazolo[1,5- a]pyridin-6-yl]-piperidin-3-yl}- terephthalamic acid | 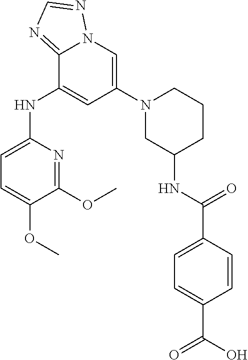 |
| I-11 | 2-Methoxy-4-(3-{8-[6-(2-methyl- pyrrolidin-1-yl)-pyridin-2-ylamino]- [1,2,4]triazolo[1,5-a]pyridin-6-yl}- benzoylamino)-benzoic acid | 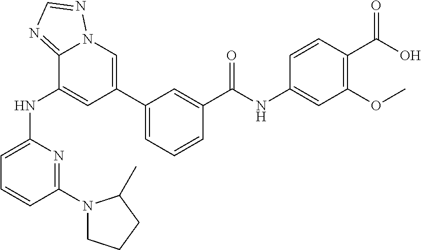 |
| I-12 | 4-{8-[6-(2-Methyl-pyrrolidin-1-yl)- pyridin-2-ylamino]- [1,2,4]triazolo[1,5-a]pyridin-6-yl}- benzoic acid | 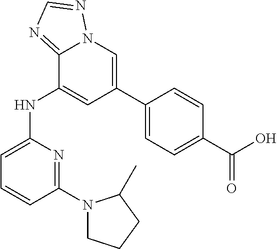 |
| I-13 | 3-{8-[6-(2-Methyl-pyrrolidin-1-yl)- pyridin-2-ylamino]- [1,2,4]triazolo[1,5-a]pyridin-6-yl}- benzoic acid methyl ester | 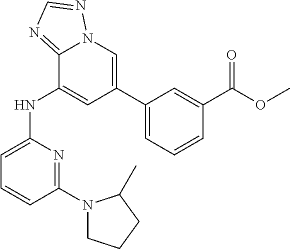 |
| I-14 | 3-{8-[6-(2-Methyl-pyrrolidin-1-yl)- pyridin-2-ylamino]- [1,2,4]triazolo[1,5-a]pyridin-6-yl}- benzoic acid | 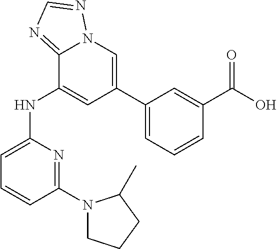 |
| I-15 | 4-({1-[8-(5,6-Dimethoxy-pyridin-2- ylamino)-[1,2,4]triazolo[1,5- a]pyridin-6-yl]-piperidine-3- carbonyl}-amino)-benzoic acid | 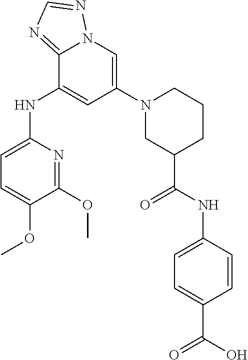 |
| I-16 | 3-{8-[(3aS,6aS)-6-(Hexahydro- cyclopenta[b]pyrrol-1-yl)-pyridin-2- ylamino]-[1,2,4]triazolo[1,5- a]pyridin-6-yl}-benzamide | 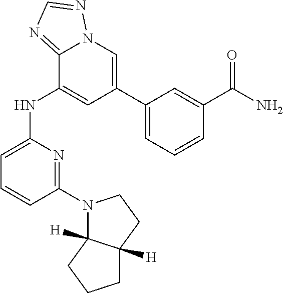 |
| I-17 | 4-[8-(5,6-Dimethoxy-pyridin-2- ylamino)-[1,2,4]triazolo[1,5- a]pyridin-6-yl]-N-(2-dimethylamino- ethyl)-benzamide | 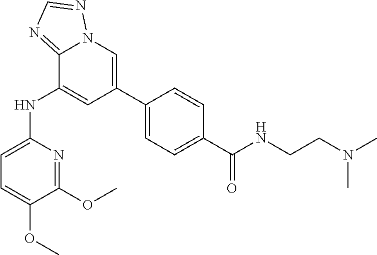 |
| I-18 | 4-[8-(5,6-Dimethoxy-pyridin-2- ylamino)-[1,2,4]triazolo[1,5- a]pyridin-6-yl]-benzamide | 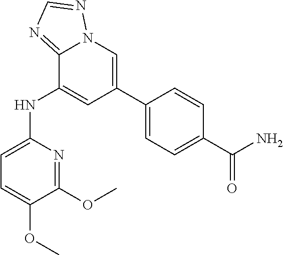 |
| I-19 | 4-{8-[6-((S)-2-Methyl-pyrrolidin-1- yl)-pyridin-2-ylamino]- [1,2,4]triazolo[1,5-a]pyridin-6-yl}- benzamide | 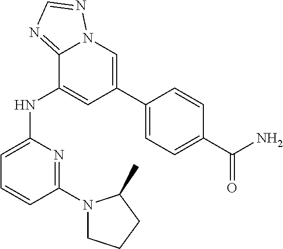 |
| I-20 | {3-[8-(5,6-Dimethoxy-pyridin-2- ylamino)-[1,2,4]triazolo[1,5- a]pyridin-6-yl]-phenyl}-methanol | 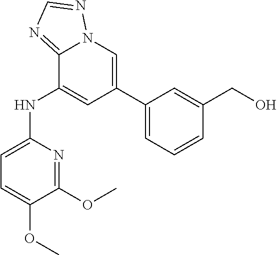 |
| I-21 | N-(2-Dimethylamino-ethyl)-4-{8-[6- ((S)-2-methyl-pyrrolidin-1-yl)- pyridin-2-ylamino]- [1,2,4]triazolo[1,5-a]pyridin-6-yl}- benzamide; compound with trifluoro- acetic acid | 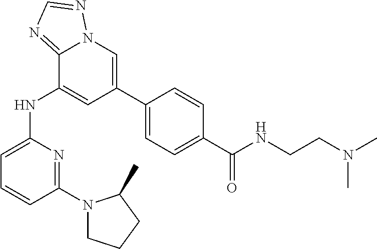 |
| I-22 | (3-{8-[6-((S)-2-Methyl-pyrrolidin-1- yl)-pyridin-2-ylamino]- [1,2,4]triazolo[1,5-a]pyridin-6-yl}- phenyl)-methanol | 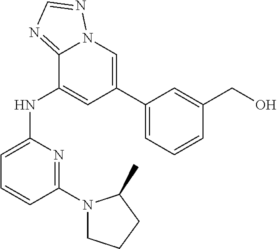 |
| I-23 | 2-(3-{8-[6-((S)-2-Methyl-pyrrolidin- 1-yl)-pyridin-2-ylamino]- [1,2,4]triazolo[1,5-a]pyridin-6-yl}- benzylamino)-ethanol | 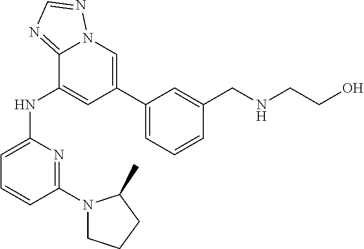 |
| I-24 | 3-{8-[6-((S)-2-Methyl-pyrrolidin-1- yl)-pyridin-2-ylamino]- [1,2,4]triazolo[1,5-a]pyridin-6-yl}- benzamide | 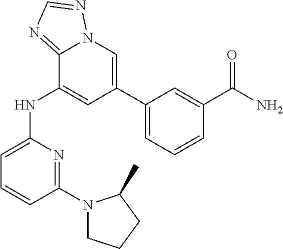 |
| I-25 | [6-((S)-2-Methyl-pyrrolidin-1-yl)- pyridin-2-yl]-{6-[3-(piperidin-4- ylaminomethyl)-phenyl]- [1,2,4]triazolo[1,5-a]pyridin-8-yl}- amine | 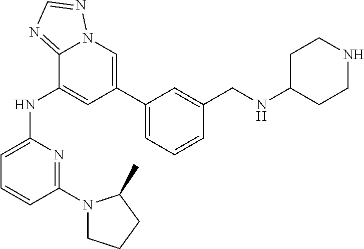 |

### Synthesis

### General Schemes

In the general schemes presented below, each X—Y—R2 can be independently C(═O)NHR, C(═O)R, C(═O)OR, OR, NHC(═O)R, CH₂NHR, lower alkyl, hydroxy lower alkyl, or hydroxy lower alkyl amino, wherein each R can be independently H, or R′, wherein each R′ can be independently lower alkyl, heterocycloalkyl, phenyl, heteroaryl, heteroaryl lower alkyl, or bicyclic heteroaryl, optionally substituted with one or more R″, wherein each R″ can be independently hydroxy, lower alkyl amido, carboxy, oxo, lower alkoxy, lower alkyl amino, or lower dialkyl amino, each R1 can be pyridinyl optionally substituted with one or more Y, wherein each Y can be independently lower alkyl, lower alkoxy, lower haloalkyl, lower hydroxyalkyl, heterocycloalkyl, optionally substituted with lower alkyl.

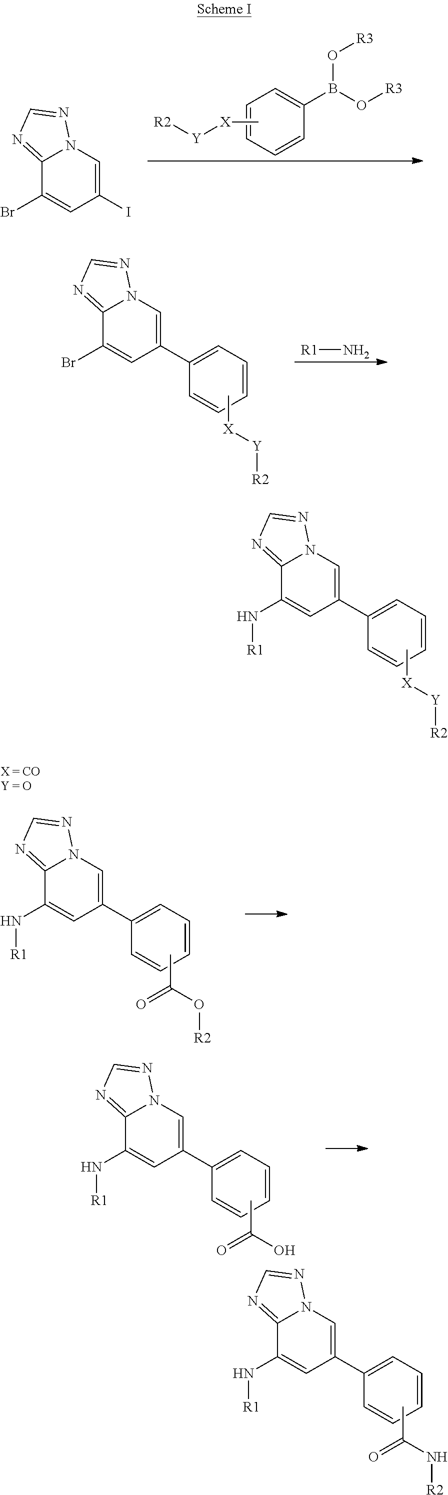

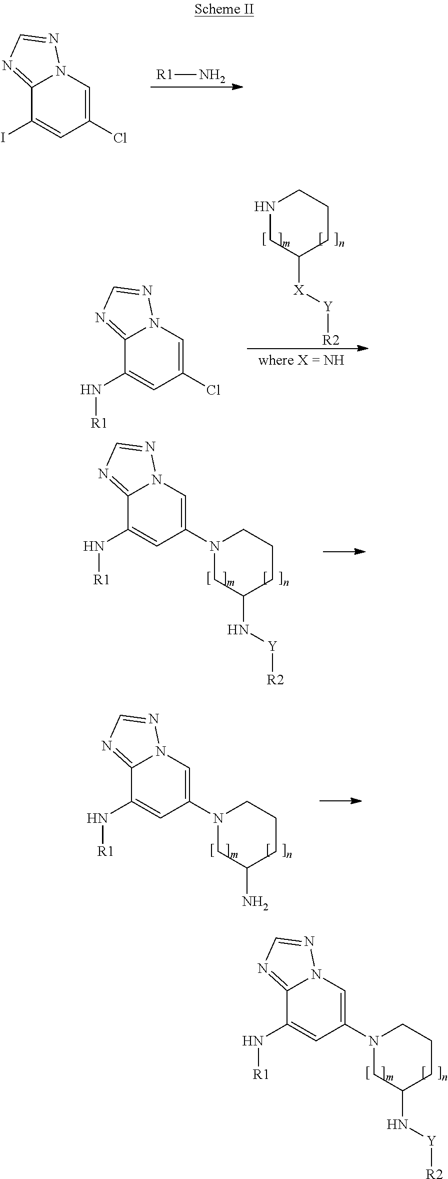

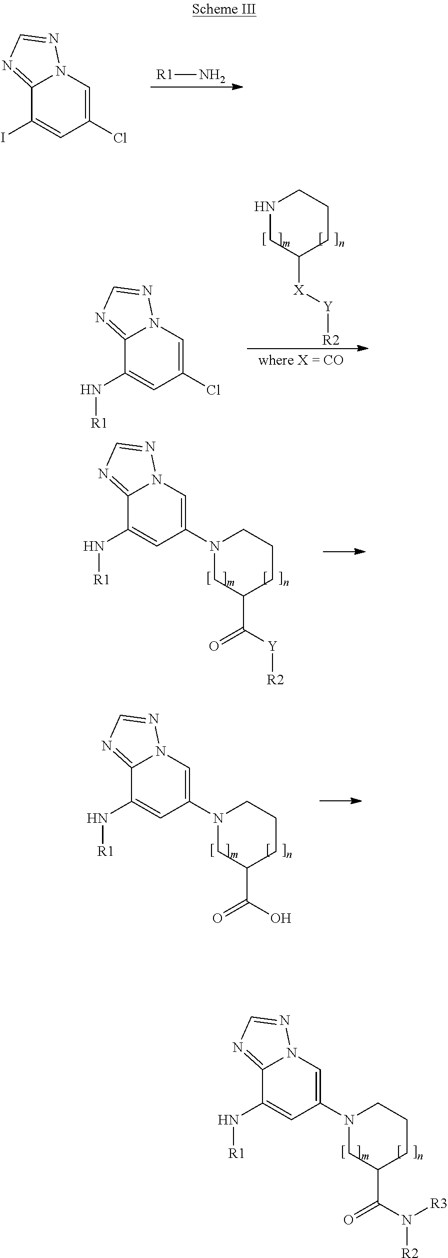

### Pharmaceutical Compositions and Administration

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Formulations

Pharmaceutical preparations for delivery by various routes are formulated as shown in the following Tables. “Active ingredient” or “Active compound” as used in the Tables means one or more of the Compounds of Formula I.

### Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

### Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

### Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

### Parenteral Formulation

| Ingredient | % wt./wt. | |
|---|---|---|
| Active ingredient | 0.25 | g |
| Sodium Chloride | qs to make isotonic | |
| Water for injection | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### Suppository Formulation

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### Topical Formulation

| Ingredients | Grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 h.

Indications and Methods of Treatment The compounds described herein are kinase inhibitors, in particular SYK inhibitors. These inhibitors can be useful for treating one or more diseases responsive to kinase inhibition, including diseases responsive to SYK inhibition and/or inhibition of B-cell proliferation, in mammals. Without wishing to be bound to any particular theory, it is believed that the interaction of the compounds of the invention with SYK results in the inhibition of SYK activity and thus in the pharmaceutical utility of these compounds. Accordingly, the invention includes a method of treating a mammal, for instance a human, having a disease responsive to inhibition of SYK activity, and/or inhibiting B-cell proliferation, comprising administrating to the mammal having such a disease, an effective amount of at least one chemical entity provided herein. An effective concentration may be ascertained experimentally, for example by assaying blood concentration of the compound, or theoretically, by calculating bioavailability. Other kinases that may be affected in addition to SYK include, but are not limited to, other tyrosine kinases and serine/threonine kinases.

Kinases play notable roles in signaling pathways controlling fundamental cellular processes such as proliferation, differentiation, and death (apoptosis). Abnormal kinase activity has been implicated in a wide range of diseases, including multiple cancers, autoimmune and/or inflammatory diseases, and acute inflammatory reactions. The multifaceted role of kinases in key cell signaling pathways provides a significant opportunity to identify novel drugs targeting kinases and signaling pathways.

The application provides a method for treating an inflammatory or autoimmune condition comprising administering to a patient in need thereof a therapeutically effective amount of the compound of Formula I.

The application provides the above method, further comprising administering an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, or an agent for treating immunodeficiency disorders.

The application provides a method for treating an inflammatory condition comprising administering to a patient in need thereof a therapeutically effective amount of the compound of Formula I.

The application provides a method for treating rheumatoid arthritis comprising administering to a patient in need thereof a therapeutically effective amount of the compound of Formula I.

The application provides a method for treating asthma comprising administering to a patient in need thereof a therapeutically effective amount of the compound of Formula I.

The application provides a method for treating an immune disorder including lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease, and Leukemia, comprising administering to a patient in need thereof a therapeutically effective amount of the compound of Formula I.

The application provides a method for treating an inflammatory condition comprising co-administering to a patient in need thereof a therapeutically effective amount of an anti-inflammatory compound in combination with the compound of Formula I.

The application provides a method for treating an immune disorder comprising co-administering to a patient in need thereof a therapeutically effective amount of an immunosuppressant compound in combination with the compound of Formula I.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), 2,2′-bis(diphenylphosphino)-1,1′-binaphthyl (BINAP), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), 2,3-Dichloro-5,6-dicyano-1,4-benzoquinone (DDQ), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), 2-ethoxy-1-ethoxycarbonyl-1,2-dihydroquinoline (EEDQ), ethyl (Et), ethyl acetate (EtOAc), ethanol (EtOH), 2-ethoxy-2H-quinoline-1-carboxylic acid ethyl ester (EEDQ), diethyl ether (Et₂O), ethyl isopropyl ether (EtOiPr), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), isopropylmagnesium chloride (iPrMgCl), hexamethyl disilazane (HMDS), liquid chromatography mass spectrometry (LCMS), lithium hexamethyl disilazane (LiHMDS), meta-chloroperoxybenzoic acid (m-CPBA), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), methyl tetrahydrofuran (MeTHF), N-bromosuccinimide (NBS), n-Butyllithium (nBuLi), N-carboxyanhydride (NCA), N-chlorosuccinimide (NCS), N-methylmorpholine (NMM), N-methylpyrrolidone (NMP), pyridinium chlorochromate (PCC), Dichloro-((bis-diphenylphosphino)ferrocenyl) palladium(II) (Pd(dppf)Cl₂), palladium(II) acetate (Pd(OAc)₂), tris(dibenzylideneacetone)dipalladium(0) (Pd₂(dba)₃), pyridinium dichromate (PDC), phenyl (Ph), propyl (Pr), iso-propyl (i-Pr), pounds per square inch (psi), pyridine (pyr), 1,2,3,4,5-Pentaphenyl-1′-(di-tert-butylphosphino)ferrocene (Q-Phos), room temperature (ambient temperature, rt or RT), sec-Butyllithium (sBuLi), tert-butyldimethylsilyl or t-BuMe₂Si (TBDMS), tetra-n-butylammonium fluoride (TBAF), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), and N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Conditions.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. The preceding abbreviations may be used in the Preparations and Examples. All names were generated using Autonom or ChemDraw.

The following preparations and examples are given to enable those skilled in the art to more clearly understand and to practice the present invention. They should not be considered as limiting the scope of the invention, but merely as being illustrative and representative thereof.

### Preparative Examples

### Example 1

### 4-(3-(8-(5,6-Dimethoxypyridin-2-ylamino)-[1,2,4]triazolo[1,5-a]pyridin-6-yl)benzamido)-2-methoxybenzoic acid

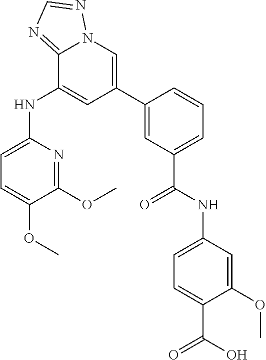

### Step 1

### Methyl-2-methoxy-4-(3-(4,4,5,5-tetramethyl-1,3,2-dioxaborolan-2-yl)benzamido)benzoate

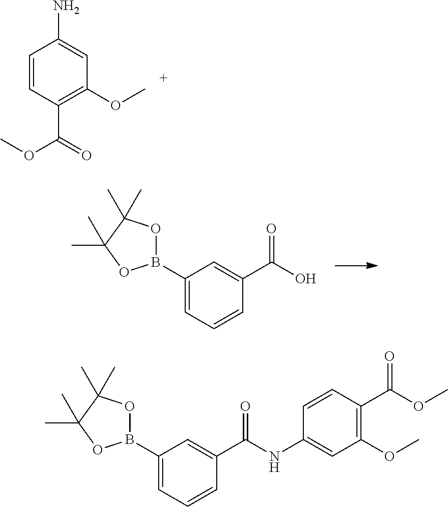

### Procedure:

A mixture of methyl 4-amino-2-methoxybenzoate (453 mg, 2.5 mmol), 3-(4,4,5,5-tetramethyl-1,3,2-dioxaborolan-2-yl)benzoic acid (1.24 g, 5 mmol), EDCI (0.96 g, 5 mmol) and DMAP (0.61 g, 5 mmol) in 10 mL of DMF was stirred at room temperature for 36 h. The mixture was poured into water and extracted with EtOAc (3×10 mL). The combined organic layers were washed with brine and dried over Na₂SO₄. After filtration and concentration, the residue was purified by column chromatography (silica gel, 200-300 mesh, petroleum ether/EtOAc 5:1, v/v) to give methyl 2-methoxy-4-(3-(4,4,5,5-tetramethyl-1,3,2-dioxaborolan-2-yl)benzamido)benzoate (240 mg, 18.5%). ¹H NMR (300 MHz, CD₃OD): δ 8.31 (s, 1H), 8.05-8.02 (m, 1H), 7.97-7.94 (m, 1H), 7.82 (d, 1H, J=6.7 Hz), 7.72 (d, 1H, J=2.1 Hz), 7.54 (t, 1H, J=7.7 Hz), 7.37 (dd, 1H, J₁=8.7 Hz, J₂=2.1 Hz), 3.32 (s, 3H), 3.31 (s, 3H), 1.38 (s, 12H). LC/MS: 412 [M+H]⁺, 844.9 [2M+Na]⁺.

### Step 2

### Methyl-4-(3-(8-bromo-[1,2,4]-triazolo[1,5-a]pyridin-6-yl)benzamido)-2-methoxybenzoate

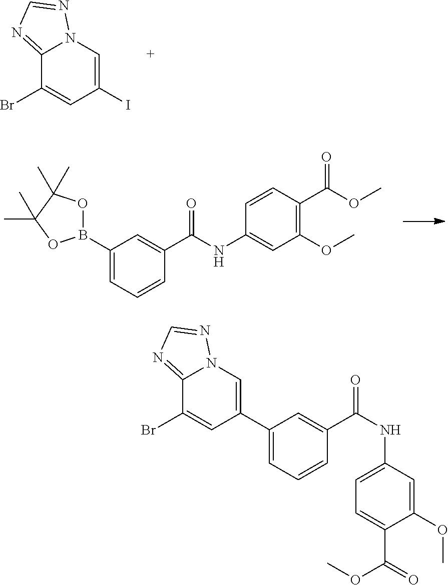

### Procedure:

To a stirred solution of 8-bromo-6-iodo-[1,2,4]triazolo[1,5-a]pyridine (172 mg, 0.53 mmol), methyl 2-methoxy-4-(3-(4,4,5,5-tetramethyl-1,3,2-dioxaborolan-2-yl)-benzamido)benzoate (240 mg, 0.584 mmol) and sodium carbonate (170 mg, 1.6 mmol) in water (2 mL) and dioxane (20 mL) at room temperature and under nitrogen was added Pd(PPh₃)₄ (32 mg, 0.0266 mmol) in one portion. The reaction mixture was degassed with bubbling nitrogen for 5 minutes. The final mixture was heated at reflux for 16 h then cooled to room temperature. The solvent was evaporated under reduced pressure then the residue was diluted with EtOAc (20 mL) and water (20 mL). The organic layers were separtated and dried over Na₂SO₄. After filtration and concentration, the residue was purified by column chromatography (silica gel, 200-300 mesh, petroleum ether/EtOAc 5:1, v/v) to give methyl 4-(3-(8-bromo-[1,2,4]triazolo[1,5-a]pyridin-6-yl)benzamido)-2-methoxybenzoate (188 mg, 39%) as a white solid. ¹H NMR (300 MHz, CDCl₃): δ 8.78 (d, 1H, J=1.5 Hz), 8.40 (s, 1H), 8.28 (brs, 1H), 8.13 (t, 1H, J=1.8 Hz), 8.04 (d, 1H, J=1.5 Hz), 7.95-7.92 (m, 1H), 7.86 (d, 1H, J=8.4 Hz), 7.82 (d, 1H, J=1.8 Hz), 7.78-7.75 (m, 1H), 7.04 (dd, 1H, J₁=8.7 Hz, J₂=1.8 Hz), 3.96 (s, 3H), 3.88 (s, 3H).

### Step 3

### Methyl-4-(3-(8-(5,6-dimethoxypyridin-2-ylamino)-[1,2,4]-triazolo[1,5-a]pyridin-6-yl)benzamido)-2-methoxybenzoate

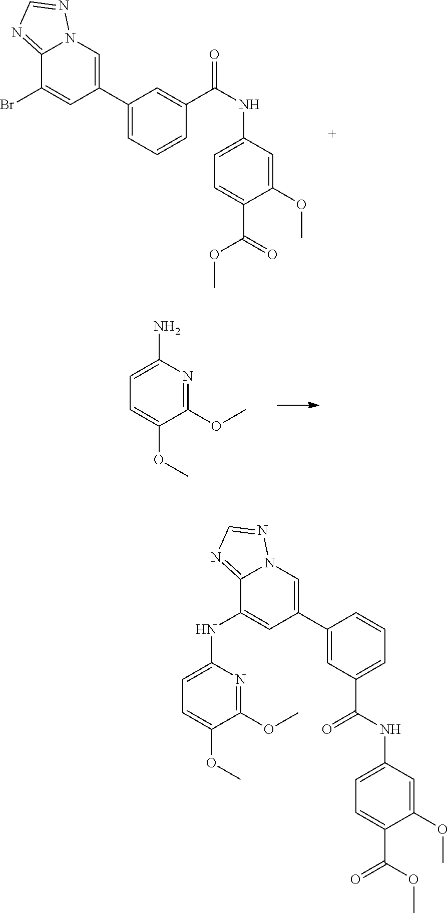

### Procedure:

To a stirred solution of methyl 4-(3-(8-bromo-[1,2,4]triazolo[1,5-a]pyridin-6-yl)benzamido)-2-methoxybenzoate (188 mg, 0.391 mmol), 5,6-dimethoxypyridin-2-amine (73 mg, 0.47 mmol), X-Phos (19 mg, 0.039 mmol) and Cs₂CO₃ (255 mg, 0.78 mmol) in dioxane (10 mL) at room temperature and under nitrogen was added Pd₂(dba)₃ (45 mg, 0.078 mmol) in a single portion. After the addition, the reaction mixture was degassed by bubbling nitrogen for 5 minutes. The final mixture was heated at 100° C. for 16 h then cooled and the solvent evaporated under reduced pressure. The residue was diluted with EtOAc (10 mL) and water (10 mL). The organic layer was separated and dried over Na₂SO₄. After filtration and concentration, the residue was purified by column chromatography (silica gel, 200-300 mesh, petroleum ether/EtOAc 5:1, v/v) to give methyl 4-(3-(8-(5,6-dimethoxypyridin-2-ylamino)-[1,2,4]triazolo[1,5-a]pyridin-6-yl)benzamido)-2-methoxybenzoate (140 mg, 65%). ¹H NMR (300 MHz, CDCl₃): δ 8.73 (d, 1H, J=1.5 Hz), 8.35 (d, 1H, J=1.8 Hz), 8.35 (s, 1H), 8.18 (t, 1H, J=1.8 Hz), 8.08 (brs, 1H), 7.88 (s, 1H), 7.86 (s, 1H), 7.82-7.80 (m, 2H), 7.63 (d, 1H, J=7.8 Hz), 7.56 (s, 1H), 7.14 (d, 1H, J=8.4 Hz), 7.01 (dd, 1H, J₁=8.7 Hz, J₂=1.8 Hz), 6.48 (d, 1H, J=8.4 Hz), 4.10 (s, 3H), 3.97 (s, 3H), 3.89 (s, 3H), 3.87 (s, 3H). LC/MS: 555.2 [M+H]⁺.

### Step 4

### 4-(3-(8-(5,6-Dimethoxypyridin-2-ylamino)-[1,2,4]-triazolo[1,5-a]pyridin-6-yl)benzamido)-2-methoxybenzoic acid

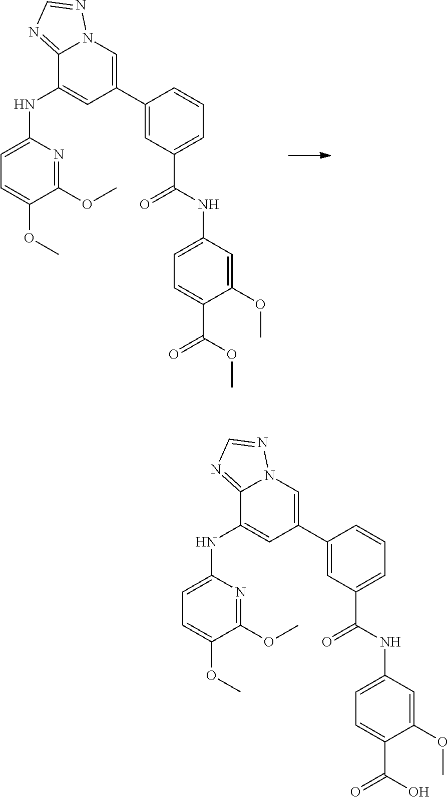

### Procedure:

To a stirred solution of methyl 4-(3-(8-(5,6-dimethoxypyridin-2-ylamino)-[1,2,4]triazolo[1,5-a]pyridin-6-yl)benzamido)-2-methoxybenzoate (140 mg, 0.25 mmol) in methanol (10 mL) and THF (10 mL) was added a solution of 1 N NaOH (10 mL) at room temperature. After 15 h, the solvent was evaporated under reduced pressure. The residue was diluted with water and adjusted to pH to 2-3 with 1N HCl). The suspension formed was filtered, washed with MeOH (3×5 mL) and dried to give 4-(3-(8-(5,6-dimethoxypyridin-2-ylamino)-[1,2,4]triazolo[1,5-a]pyridin-6-yl)benzamido)-2-methoxybenzoic acid (45 mg, 33%) as white solid. ¹H NMR (300 MHz, DMSO): δ 12.35 (s, 1H), 10.56 (s, 1H), 9.35 (s, 1H), 8.95 (s, 1H), 8.90 (s, 1H), 8.55 (s, 1H), 8.36 (s, 1H), 8.05-7.99 (m, 2H), 7.77-7.68 (m, 3H), 7.51 (d, 1H, J=6.9 Hz), 7.38 (d, 1H, J=8.7 Hz), 7.00 (d, 1H, J=8.4 Hz), 3.97 (s, 3H), 3.85 (s, 3H), 3.75 (s, 3H). LC/MS: 541 [M+H]⁺, t_{R}=1.58 min. HPLC: 95.63% at 214 nm, 95.18% at 254 nm.

### Example 2

### 4-(1-(8-(5,6-Dimethoxypyridin-2-ylamino)-[1,2,4]triazolo[1,5-a]pyridin-6-yl)piperidin-3-ylcarbamoyl)benzoic acid

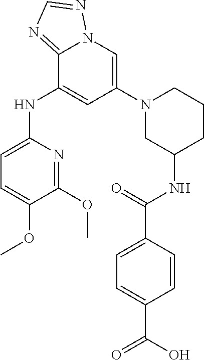

### Step 1

### 5-Chloro-3-iodopyridin-2-amine

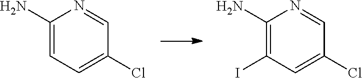

### Procedure:

To a solution of 5-chloropyridin-2-amine (10.0 g, 78.1 mmol) and iodine (39.7 g, 156.2 mmol) in EtOH (500 mL), Ag₂SO₄ (48.7 g, 156.2 mmol) was added portion-wise at ambient temperature. The final mixture was then stirred overnight at ambient temperature. The formed solid (AgI) was removed by filtration; the filtrate was evaporated to dryness. The residue was dissolved in DCM (1 L), washed by saturated Na₂S₂O₃ (aq., 2×50 ml), brine (2×50 mL), dried over Na₂SO₄ and concentrated. The crude product was purified by column chromatography (silica gel, 200-300 mesh, dichloromethane as eluent) to give 5-chloro-3-iodopyridin-2-amine (13.5 g, 68%) as yellow solid. ¹H NMR (300 MHz, CDCl₃): δ 7.99 (brs, 1H), 7.83 (s, 1H), 5.00-4.94 (m, 2H). LC/MS: 254.9 [M+H]⁺.

### Step 2

### (Z)—N′-(5-Chloro-3-iodopyridin-2-yl)-N-hydroxyformimidamide

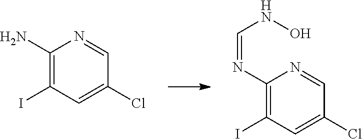

### Procedure:

To a solution of 5-chloro-3-iodopyridin-2-amine (800 mg, 3.4 mmol) in isopropanol (20 mL), N,N-dimethylformamide dimethyl acetal (747 mg, 6.28 mmol) was added dropwise at ambient temperature. The resulting mixture was stirred at 100° C. for 5 h then the mixture was cooled to 50-60° C. and hydroxylamine hydrochloride (650 mg, 9.42 mmol) and NaHCO₃ (932 mg, 11 mmol) was added in a single portion. The final mixture was stirred at 55° C. for 16 h. After cooling to 30° C., the excess solvent was removed under reduced pressure and the crude residue purified by column chromatography (silica gel, 200-300 mesh, THF) to give the desired (Z)—N′-(5-chloro-3-iodopyridin-2-yl)-N-hydroxyformimidamide (900 mg, 88%) as yellow solid. ¹H NMR (300 MHz, CDCl₃): δ 8.22-8.12 (brs, 3H), 8.04 (d, 1H, J=9.9 Hz), 7.98 (d, 1H, J=2.4 Hz). LC/MS: 297.9 [M+H]⁺.

### Step 3

### 6-Chloro-8-iodo-[1,2,4]-triazolo[1,5-a]pyridine

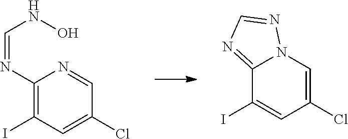

### Procedure:

To a solution of (Z)—N′-(5-chloro-3-iodopyridin-2-yl)-N-hydroxyformimidamide (900 mg, 3.0 mmol) in THF (20 mL), 2,2,2-trifluoroacetic anhydride (5 mL) was added dropwise at 0° C. (ice-water bath). The final mixture was stirred at ambient temperature for 16 h. The excess solvent was removed under reduced pressure then the crude residue was dissolved in THF (50 mL), washed by saturated NaHCO₃ (aq., 2×5 mL) and dried over Na₂SO₄. The crude product was purified by column chromatography (silica gel, 200-300 mesh, THF/petroleum ether 1:4, v/v) to give the desired 6-chloro-8-iodo-[1,2,4]triazolo[1,5-a]pyridine (720 mg, 85.2%) as yellow solid. ¹H NMR (300 MHz, CDCl₃): δ 8.62 (d, 1H, J=1.8 Hz), 8.36 (s, 1H), 7.98 (d, 1H, J=1.8 Hz). LC/MS: 279.9 [M+H]⁺.

### Step 4

### 6-Chloro-N-(5,6-dimethoxypyridin-2-yl)-[1,2,4]-triazolo[1,5-a]-yridine-8-amine

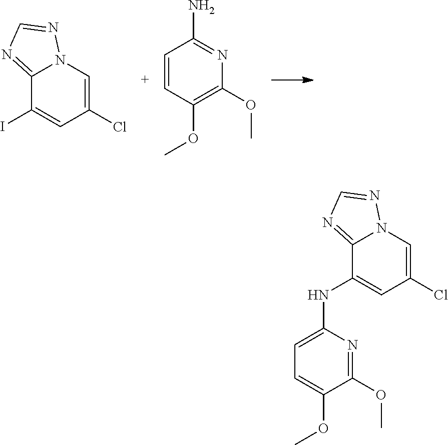

### Procedure:

A mixture of 6-chloro-8-iodo-[1,2,4]triazolo[1,5-a]pyridine (500 mg, 1.8 mmol), 5,6-dimethoxypyridin-2-amine (400 mg, 2.6 mmol), Pd(dba)₂ (100 mg, 0.17 mmol), X-Phos (100 mg, 0.21 mmol) and Cs₂CO₃ (1200 mg, 3.6 mmol) in xylene (50 mL) were stirred at reflux for 8 h and then at 95° C. for 36 h. The solvent was removed under reduced pressure and then the residue was purified by chromatography (silica, EtOAc/petroleum ether 1:4, v/v) to give 6-chloro-N-(5,6-dimethoxypyridin-2-yl)-[1,2,4]triazolo[1,5-a]-yridine-8-amine (380 mg, 69%) as a yellow solid. ¹H NMR (300 MHz, DMSO): δ 9.54 (s, 1H), 8.70 (d, 1H, J=2.1 Hz), 8.56 (d, 1H, J=1.8 Hz), 8.51 (s, 1H), 7.37 (d, 1H, J=8.7 Hz), 6.97 (d, 1H, J=8.1 Hz), 3.95 (s, 3H), 3.74 (s, 3H). LC/MS: 306 [M+H]⁺.

### Step 5

### tert-Butyl 1-(8-(5,6-dimethoxypyridin-2-ylamino)-[1,2,4]triazolo[1,5-a]pyridin-6-yl)piperidin-3-ylcarbamate

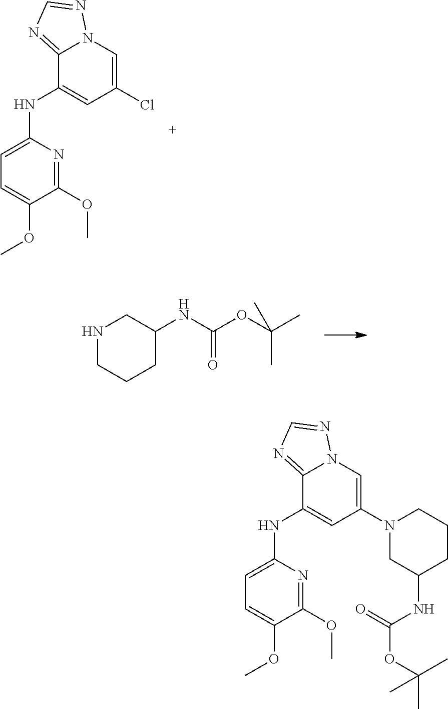

### Procedure:

A mixture of 6-chloro-N-(5,6-dimethoxypyridin-2-yl)-[1,2,4]triazolo[1,5-a]pyridin-8-amine (120 mg, 0.39 mmol), tert-butyl piperidin-3-ylcarbamate (156 mg, 0.78 mmol), Pd(dba)₂ (30 mg, 0.05 mmol), X-Phos (30 mg, 0.06 mmol) and Cs₂CO₃ (254 mg, 0.78 mmol) in xylene (30 mL) was stirred at 140° C. for 5 h. The solvent was removed under reduced pressure and then the residue was filtered through a plug of silica gel. The filter cake was washed with EtOAc/petroleum ether (1:3 to 1:1, v/v) to give crude tert-butyl 1-(8-(5,6-dimethoxypyridin-2-ylamino)-[1,2,4]triazolo[1,5-a]pyridin-6-yl)piperidin-3-ylcarbamate (32 mg) as a yellow solid that was used directly without further purification. LC/MS: 470 [M+H]⁺.

### Step 6

### 6-(3-Aminopiperidin-1-yl)-N-(5,6-dimethoxypyridin-2-yl)-[1,2,4]triazolo[1,5-a]pyridin-8-amine hydrochloride

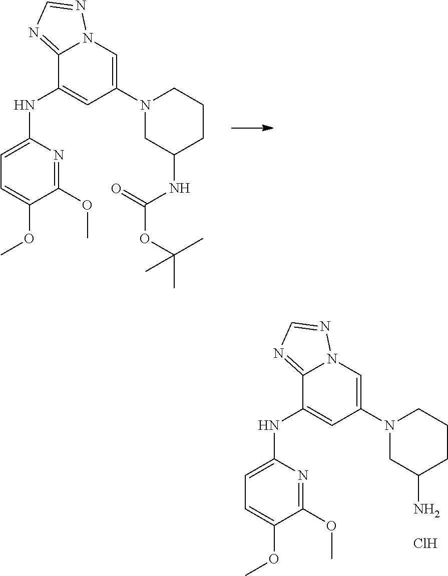

### Procedure:

A solution of tert-butyl 1-(8-(5,6-dimethoxypyridin-2-ylamino)-[1,2,4]triazolo[1,5-a]pyridin-6-yl)piperidin-3-ylcarbamate (30 mg) in 4M HCl in dioxane (30 mL) was stirred at room temperature for 1 h. The excess solvent was removed under reduced pressure to give crude 6-(3-aminopiperidin-1-yl)-N-(5,6-dimethoxypyridin-2-yl)-[1,2,4]triazolo[1,5-a]pyridin-8-amine as hydrochloride salt (38 mg), which was used directly in the next step without further purification. LC/MS: 370 [M+H]⁺.

### Step 7

### tert-Butyl 4-(1-(8-(5,6-dimethoxypyridin-2-ylamino)-[1,2,4]triazolo[1,5-a]pyridin-6-yl)piperidin-3-ylcarbamoyl)benzoate

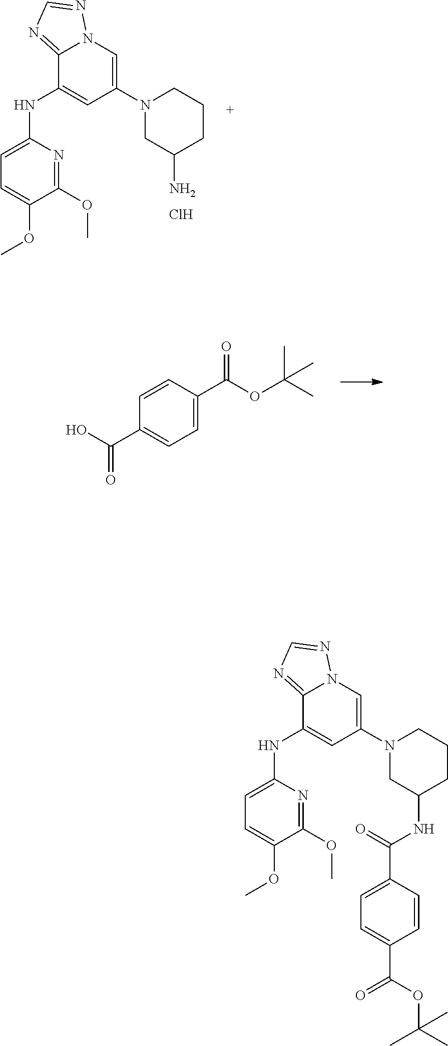

### Procedure:

A mixture of 6-(3-aminopiperidin-1-yl)-N-(5,6-dimethoxypyridin-2-yl)-[1,2,4]triazolo[1,5-a]pyridin-8-amine hydrochloride (38 mg, crude), 4-(tert-butoxycarbonyl)benzoic acid (38 mg, 0.17 mmol), EDCI (65 mg, 0.34 mmol) and 1-methyl-1H-imidazole (70 mg, 0.85 mmol) in DCM (30 mL) were stirred at room temperature for 18 h. The excess solvent was removed under reduced pressure then the residue was filtered through a plug of silica gel and the filter cake washed with EtOAc/petroleum ether (1:3, v/v) to give crude tert-butyl 4-(1-(8-(5,6-dimethoxypyridin-2-ylamino)-[1,2,4]triazolo[1,5-a]pyridin-6-yl)piperidin-3-ylcarbamoyl)benzoate (20 mg) as a yellow solid. This was used directly in the next step without further purifiacation. LC/MS: 574 [M+H]⁺.

### Step 8

### 4-(1-(8-(5,6-Dimethoxypyridin-2-ylamino)-[1,2,4]-triazolo[1,5-a]pyridin-6-yl)piperidin-3-ylcarbamoyl)benzoic acid

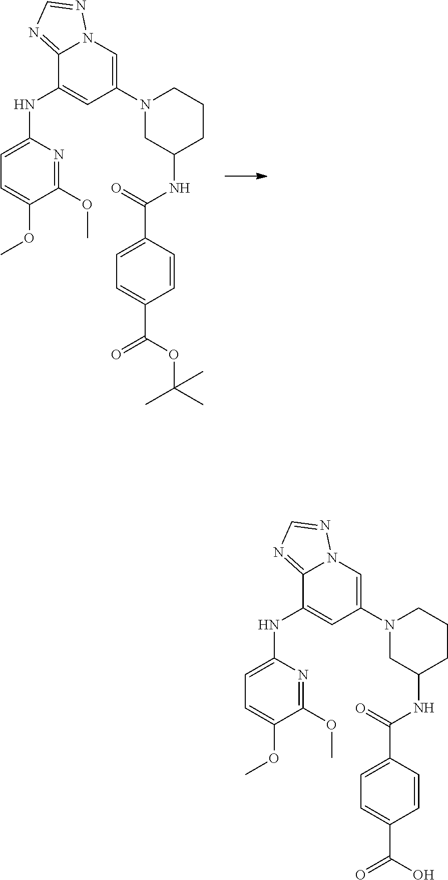

### Procedure:

A solution of tert-butyl 4-(1-(8-(5,6-dimethoxypyridin-2-ylamino)-[1,2,4]triazolo[1,5-a]pyridin-6-yl)piperidin-3-ylcarbamoyl)benzoate (20 mg, 0.035 mmol) in TFA/DCM (2 mL/4 mL) was stirred at room temperature for 4 h. The excess solvent was removed under reduced pressure. The residue was purified by preparative-HPLC (Gemini 5u C18 150×21.2 mm; inject volume: 3 ml/inj, flow rate: 20 mL/min; wavelength: 214 nm and 254 nm; gradient conditions: 15% acetonitrile/75% water (0.1% TFA, v/v) initially, proceeding to 70% acetonitrile/30% water (0.1% TFA, v/v) in a linear fashion after just 9 min) to give 4-(1-(8-(5,6-dimethoxypyridin-2-ylamino)-[1,2,4]triazolo[1,5-a]pyridin-6-yl)piperidin-3-ylcarbamoyl)benzoic acid 2,2,2-trifluoroacetate (5 mg, 28%) as a yellow solid. ¹H NMR (300 MHz, DMSO): δ 13.20 (brs, 1H), 9.05 (s, 1H), 8.54 (d, 1H, J=7.5 Hz), 8.44 (s, 1H), 8.32 (s, 1H), 8.03-7.91 (m, 5H), 7.34 (d, 1H, J=8.7 Hz), 6.93 (d, 1H, J=8.7 Hz), 4.10 (brs, 1H), 3.98 (s, 3H), 3.92 (brs, 4H), 3.79-3.75 (m, 1H), 2.75-2.61 (m, 2H), 2.00-1.84 (m, 2H), 1.67-1.56 (m, 2H). LC/MS: 518 [M+H]⁺, 516 [M−H]⁻; HPLC: 100% at 214 nm, 100% at 254 nm, t_{R}=5.51 min.

### Example 3

### 4-(1-(8-(5,6-Dimethoxypyridin-2-ylamino)-[1,2,4]triazolo[1,5-a]pyridin-6-yl)piperidine-3-carboxamido)benzoic acid hydrochloride

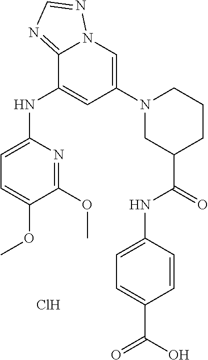

### Step 1

### Methyl 1-(8-(5,6-dimethoxypyridin-2-ylamino)-[1,2,4]triazolo[1,5-a]pyridin-6-yl)piperidine-3-carboxylate

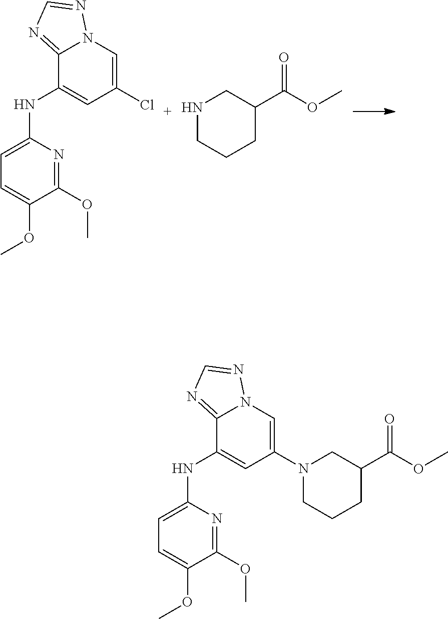

### Procedure:

A mixture of 6-chloro-N-(5,6-dimethoxypyridin-2-yl)-[1,2,4]triazolo[1,5-a]pyridin-8-amine (400 mg, 1.3 mmol), methyl piperidine-3-carboxylate (400 mg, 2.8 mmol), Pd(dba)₂ (100 mg, 0.17 mmol), X-Phos (100 mg, 0.21 mmol) and Cs₂CO₃ (800 mg, 2.5 mmol) in xylene (100 mL) was stirred at 135° C. for 8 h. The solvent was removed under reduced pressure then the residue was filtered through a plug of silica gel and the filter cake washed with EtOAc/petroleum ether (1:3 to 1:1, v/v) to give crude methyl 1-(8-(5,6-dimethoxypyridin-2-ylamino)-[1,2,4]triazolo[1,5-a]pyridin-6-yl)piperidine-3-carboxylate (160 mg, 30%) as a yellow solid. This was used directly without further purification LC/MS: 413 [M+H]⁺.

### Step 2

### 1-(8-(5,6-Dimethoxypyridin-2-ylamino)-[1,2,4]triazolo[1,5-a]pyridin-6-yl)piperidine-3-carboxylic acid

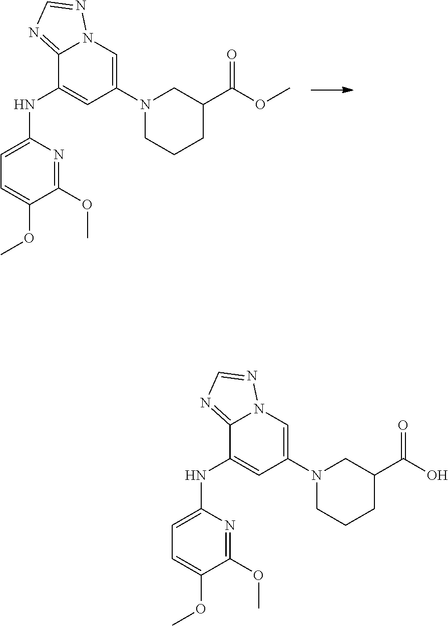

### Procedure:

A suspension of methyl 1-(8-(5,6-dimethoxypyridin-2-ylamino)-[1,2,4]triazolo[1,5-a]pyridin-6-yl)piperidine-3-carboxylate (80 mg, 0.19 mmol) and NaOH (80 mg, 2 mmol) in dioxane/H₂O (2.5 mL/2.5 mL) was stirred at room temperature for 1.5 h. The pH was adjusted to 3-4 by adding concentrated HCl. The excess solvent was removed under reduced pressure, the obtained solid residue was suspended in THF (30 mL), stirred for 10 minutes and decanted. This decanting step was repeated (2×30 mL). The combined organic solutions were concentrated under reduced pressure to give crude 1-(8-(5,6-dimethoxypyridin-2-ylamino)-[1,2,4]triazolo[1,5-a]pyridin-6-yl)piperidine-3-carboxylic acid (60 mg, 79%) as a solid residue that was used directly in the next step without further purification. LC/MS: 399 [M+H]⁺.

### Step 3

### tert-Butyl 4-(1-(8-(5,6-dimethoxypyridin-2-ylamino)-[1,2,4]triazolo[1,5-a]pyridin-6-yl)piperidine-3-carboxamido)benzoate

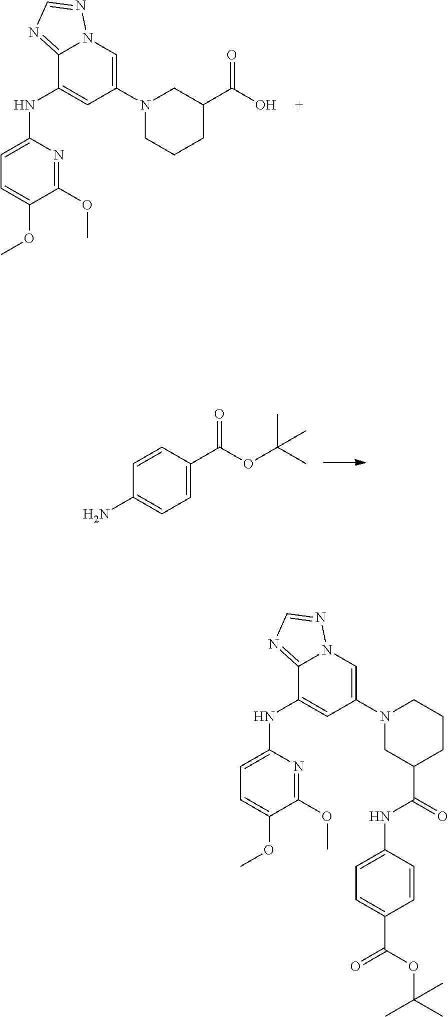

### Procedure:

A mixture of 1-(8-(5,6-dimethoxypyridin-2-ylamino)-[1,2,4]triazolo[1,5-a]pyridin-6-yl)piperidine-3-carboxylic acid (60 mg, 0.15 mmol), tert-butyl 4-aminobenzoate (60 mg, 0.31 mmol), EDCI (86 mg, 0.45 mmol) and 1-methyl-1H-imidazole (50 mg, 0.6 mmol) in DCM (25 mL) was stirred at room temperature for 15 h. The solvent was removed under reduced pressure then the residue was filtered through a plug of silica gel. The filter cake was washed with EtOAc and the combined filtrates concentrated to give crude tert-butyl 4-(1-(8-(5,6-dimethoxypyridin-2-ylamino)-[1,2,4]triazolo[1,5-a]pyridin-6-yl)piperidine-3-carboxamido)benzoate (60 mg, 67%) as a yellow oil. This was used directly without further purification. LC/MS: 574 [M+H]⁺.

### Step 4

### 4-(1-(8-(5,6-Dimethoxypyridin-2-ylamino)-[1,2,4]-triazolo[1,5-a]pyridin-6-yl)piperidine-3-carboxamido)benzoic acid hydrochloride

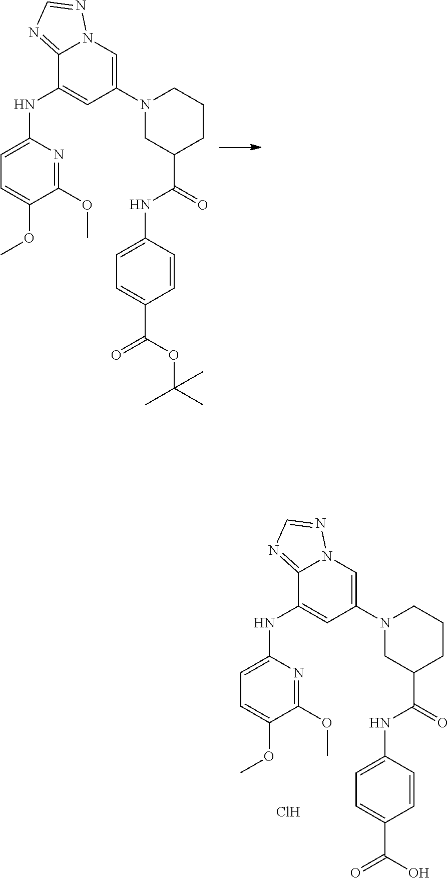

### Procedure:

A solution of tert-butyl 4-(1-(8-(5,6-dimethoxypyridin-2-ylamino)-[1,2,4]triazolo[1,5-a]pyridin-6-yl)piperidine-3-carboxamido)benzoate (60 mg, 0.1 mmol) in TFA/DCM (5 mL/10 mL) was stirred at room temperature for 18 h. The solvent was removed under reduced pressure then the residue was purified by preparative-HPLC (Gemini 5u C18 150×21.2 mm; inject volume: 3 ml/inj, flow rate: 20 mL/min; wavelength: 214 nm and 254 nm; gradient conditions: 37% acetonitrile/63% water (0.1% TFA, v/v) initially, proceeding to 50% acetonitrile/50% water (0.1% TFA, v/v) in a linear fashion after 9 min). The solution of the purified compound was adjusted to pH=2 with 1N HCl then concentrated under reduced pressure to give 4-(1-(8-(5,6-dimethoxypyridin-2-ylamino)-[1,2,4]triazolo[1,5-a]pyridin-6-yl)piperidine-3-carboxamido)benzoic acid hydrochloride (30 mg, 56%) as a white solid. ¹H NMR (300 MHz, DMSO): δ 10.32 (s, 1H, D₂O exchangeable), 9.07 (s, 1H, D₂O exchangeable), 8.44 (s, 1H), 8.37 (s, 1H), 7.99 (s, 1H, D₂O exchangeable), 7.92-7.89 (m, 2H), 7.76-7.73 (m, 2H), 7.35 (d, 1H, J=8.1 Hz), 7.17-7.00 (m, 1H, D₂O exchangeable), 6.92 (d, 1H, J=8.1 Hz), 3.91 (s, 3H), 3.81-3.61 (m, 5H), 2.92-2.74 (m, 3H), 2.28 (brs, 1H), 2.03-1.68 (m, 3H). LC/MS: 518 [M+H]⁺. HPLC: 100% at 214 nm, 100% at 254 nm, t_{R}=5.63 min.

### Example 4

### 4-(3-(8-(5,6-Dimethoxypyridin-2-ylamino)-[1,2,4]-triazolo[1,5-a]pyridin-6-yl)benzamido)benzoic acid

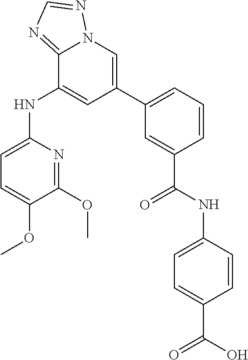

### Step 1

### tert-Butyl 4-(3-(4,4,5,5-tetramethyl-1,3,2-dioxaborolan-2-yl)benzamido)benzoate

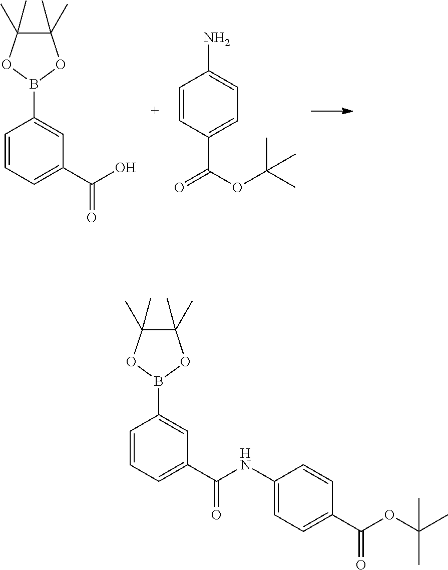

### Procedure:

A mixture of 3-(4,4,5,5-tetramethyl-1,3,2-dioxaborolan-2-yl)benzoic acid (0.8 g, 3.2 mmol), tert-butyl 4-aminobenzoate (0.74 g, 3.8 mmol), HATU (1.47 g, 3.82 mmol) and DIEA (1 g, 7.7 mmol) in DMF (10 mL) was stirred at room temperature for 24 h. The solvent was evaporated under reduced pressure and the residue was purified by column chromatography (silica gel, 200-300 mesh, ethyl acetate/petroleum ether 1:8, v/v) to give tert-butyl 4-(3-(4,4,5,5-tetramethyl-1,3,2-dioxaborolan-2-yl)benzamido)benzoate (1.04 g, 76%) as a white solid. ¹H NMR (300 MHz, DMSO): δ 10.64 (s, 1H), 8.24 (s, 1H), 8.07 (dd, 1H, J₁=7.8 Hz, J₂=1.5 Hz), 7.93-7.86 (m, 5H), 7.55 (t, 1H, J=15 Hz), 1.54 (s, 9H), 1.32 (s, 12H). LC/MS: 424.2 [M+H]⁺.

### Step 2

### tert-Butyl 4-(3-(8-bromo-[1,2,4]triazolo[1,5-a]pyridin-6-yl)benzamido)benzoate

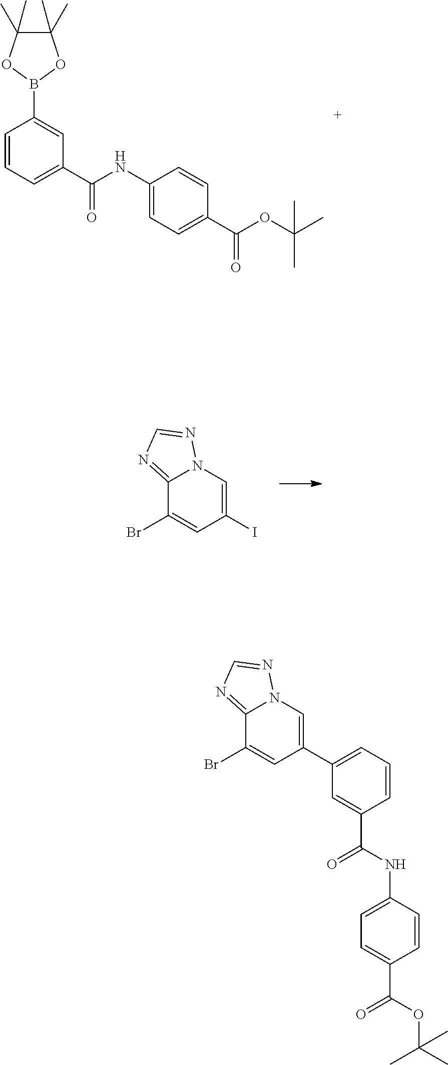

### Procedure:

To a stirred solution of tert-butyl 4-(3-(4,4,5,5-tetramethyl-1,3,2-dioxaborolan-2-yl)benzamido)benzoate (0.6 g, 1.4 mmol), 8-bromo-6-iodo-[1,2,4]triazolo[1,5-a]pyridine (0.504 g, 1.55 mmol), Na₂CO₃ (0.59 g, 5.5 mmol) and H₂O (6 mL) in dioxane (60 mL) was added Pd(PPh₃)₄ (0.176 g, 0.14 mmol) in a single portion at room temperature under nitrogen, then the reaction mixture was degassed with bubbling nitrogen for 10 minutes and then heated at 96° C. for 24 h. The excess solvent was evaporated at 40° C. under reduced pressure and the residue was filtered through a plug of silica gel. The filter cake was washed with ethyl acetate and petroleum ether (1:1, v/v) and the combined filtrates concentrated to give crude tert-butyl 4-(3-(8-bromo-[1,2,4]triazolo[1,5-a]pyridin-6-yl)benzamido)benzoate (0.3 g, 43.6%) as a solid that was used directly without further purification. LC/MS: 492.9 [M+H]⁺.

### Step 3

### tert-Butyl 4-(3-(8-(5,6-dimethoxypyridin-2-ylamino)-[1,2,4]triazolo[1,5-a]pyridin-6-yl)benzamido)benzoate

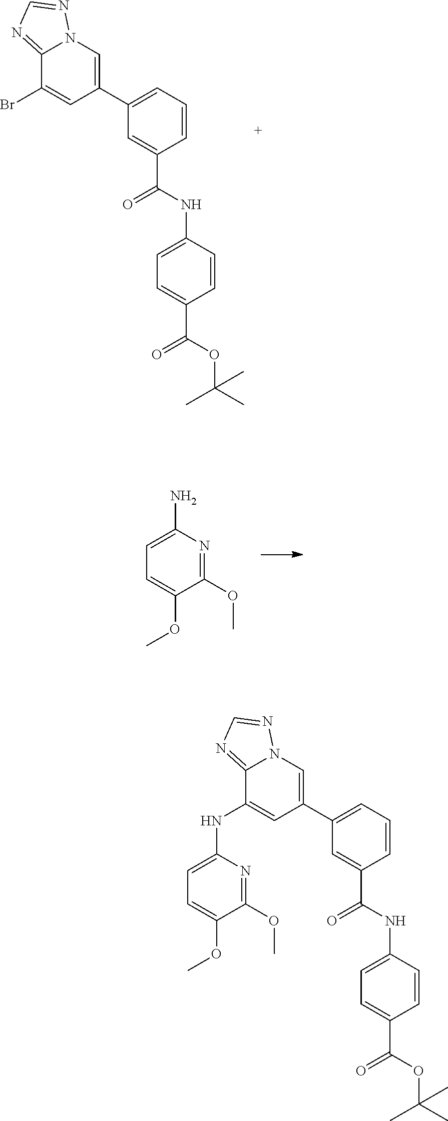

### Procedure:

A mixture of tert-butyl 4-(3-(8-bromo-[1,2,4]triazolo[1,5-a]pyridin-6-yl)benzamido)benzoate (300 mg, 0.60 mmol), 5,6-dimethoxypyridin-2-amine (140 mg, 0.91 mmol), Cs₂CO₃ (754 mg, 2.3 mmol) and X-Phos (150 mg, 0.3 mmol) in dioxane (45 mL) was added Pd₂(dba)₃ (77 mg, 0.084 mmol). The reaction mixture was degassed by bubbling nitrogen through it for 15 minutes, then was heated to 95° C. for 24 h. The mixture was cooled and excess solvent was evaporated under reduced pressure then the residue was filtered through a plug of silica gel. The filter cake was washed with ethyl acetate and petroleum ether (2:1, v/v) and the combined filtrates concentrated to give tert-butyl 4-(3-(8-(5,6-dimethoxypyridin-2-ylamino)-[1,2,4]triazolo[1,5-a]pyridin-6-yl)benzamido)benzoate (150 mg, 44.1%) as a solid. LC/MS: 567.2 [M+H]⁺.

### Step 4

### 4-(3-(8-(5,6-Dimethoxypyridin-2-ylamino)-[1,2,4]-triazolo[1,5-a]pyridin-6-yl)benzamido)benzoic acid

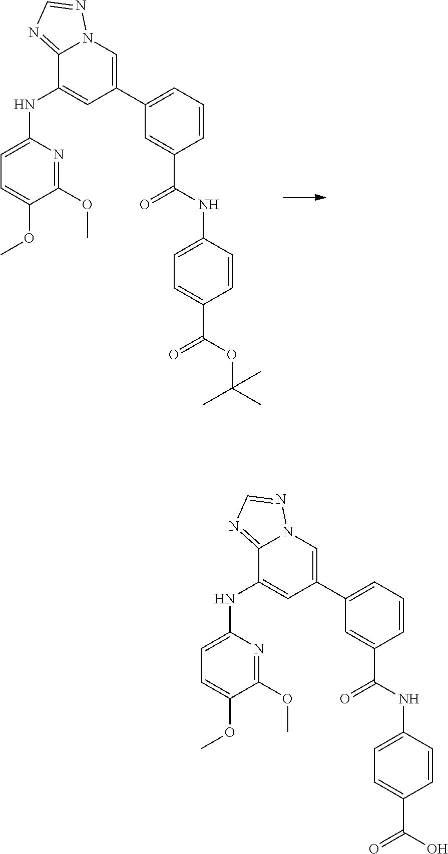

### Procedure:

To a stirred solution of tert-butyl 4-(3-(8-(5,6-dimethoxypyridin-2-ylamino)-[1,2,4]triazolo[1,5-a]pyridin-6-yl)benzamido)benzoate (76 mg, 0.134 mmol) in dichloromethane (35 mL) was added trifluoroacetic acid (5 mL) dropwise at room temperature. After 24 h the solvent was evaporated at 40° C. under reduced pressure and the residue was treated with 5 mL of methanol, and then evaporated again. The residue was suspended in 5 mL of methanol and filtered to give the desired 4-(3-(8-(5,6-dimethoxypyridin-2-ylamino)-[1,2,4]triazolo[1,5-a]pyridin-6-yl)benzamido)benzoic acid 2,2,2-trifluoroacetate (40 mg, 58.5%) as an off-white solid. ¹H NMR (300 MHz, DMSO): δ 12.77 (s, 1H), 10.62 (s, 1H), 9.36 (s, 1H), 8.96 (s, 1H), 8.90 (s, 1H), 8.55 (s, 1H), 8.36 (s, 1H), 8.05-7.93 (m, 6H), 7.40 (t, 1H, J=7.8 Hz), 7.39 (d, 1H, J=8.4 Hz), 7.01 (d, 1H, J=8.4 Hz), 3.96 (s, 1H), 3.75 (s, 1H). LC/MS: 511 [M+H]⁺, 509 [M−H]⁻. HPLC: 99.14% at 214 nm, 97.53% at 254 nm, t_{R}=6.34 min.

### Example 5

### 3-(8-(5,6-Dimethoxypyridin-2-ylamino)-[1,2,4]triazolo[1,5-a]pyridin-6-yl)-N-(4-(methylcarbamoyl)phenyl)benzamide

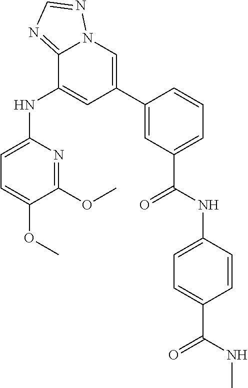

### Step 1

### N-(4-(Methylcarbamoyl)phenyl)-3-(4,4,5,5-tetramethyl-1,3,2-dioxaborolan-2-yl)benzamide

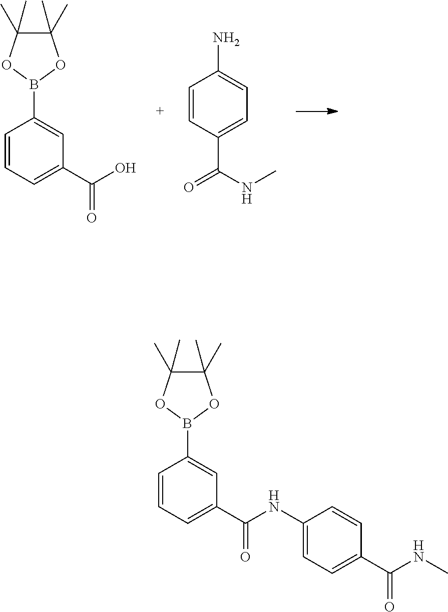

### Procedure:

A mixture of 3-(4,4,5,5-tetramethyl-1,3,2-dioxaborolan-2-yl)benzoic acid (0.8 g, 3.22 mmol), 4-amino-N-methylbenzamide (0.58 g, 3.86 mmol), HATU (1.47 g, 3.8 mmol) and DIEA (1 g, 7.7 mmol) in DMF (10 mL) was stirred at room temperature for 24 h. The solvent was evaporated at 70° C. under reduced pressure to give a crude product. It was filtered through a plug of silica gel and the filter cake washed with ethyl acetate/petroleum ether (1:1, v/v). The combined filtrates were concentrated to give crude N-(4-(methylcarbamoyl)phenyl)-3-(4,4,5,5-tetramethyl-1,3,2-dioxaborolan-2-yl)benzamide (0.56 g, 45.9%) as a white solid that was used directly without further purification. LC/MS: 381.1 [M+H]⁺.

### Step 2

### 3-(8-Bromo-[1,2,4]-triazolo[1,5-a]pyridin-6-yl)-N-(4-(methylcarbamoyl)phenyl)benzamide

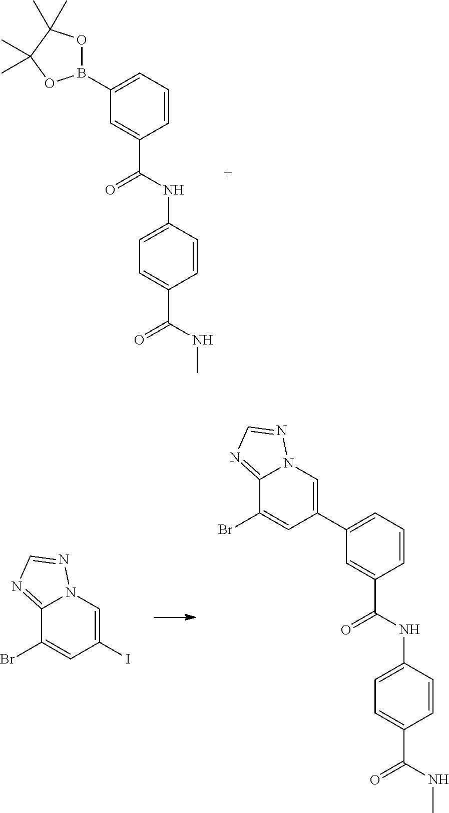

### Procedure:

To a stirred solution of N-(4-(methylcarbamoyl)phenyl)-3-(4,4,5,5-tetramethyl-1,3,2-dioxaborolan-2-yl)benzamide (0.56 g, 1.47 mmol), 8-bromo-6-iodo-[1,2,4]triazolo[1,5-a]pyridine (0.523 g, 1.62 mmol), Na₂CO₃ (0.619 g, 5.8 mmol) and H₂O (5 mL) in dioxane (50 mL) at room temperature under nitrogen was added Pd(PPh₃)₄ (0.185 g, 0.15 mmol). The reaction mixture was degassed by bubbling nitrogen for 10 minutes, and then stirred at 95° C. for 24 h. The excess solvent was evaporated and the residue was filtered through a plug of silica gel. The filter cake was washed with 5% methanol in ethyl acetate and the combined filtrates concentrated to give crude 3-(8-bromo-[1,2,4]triazolo[1,5-a]pyridin-6-yl)-N-(4-(methylcarbamoyl)phenyl)benzamide (0.53 g, 80%) as a white solid that was used directly without further purification. LC/MS: 450.0 [M+H]⁺.

### Step 3

### 3-(8-(5,6-Dimethoxypyridin-2-ylamino)-[1,2,4]triazolo[1,5-a]pyridin-6-yl)-N-(4-(methylcarbamoyl)phenyl)benzamide

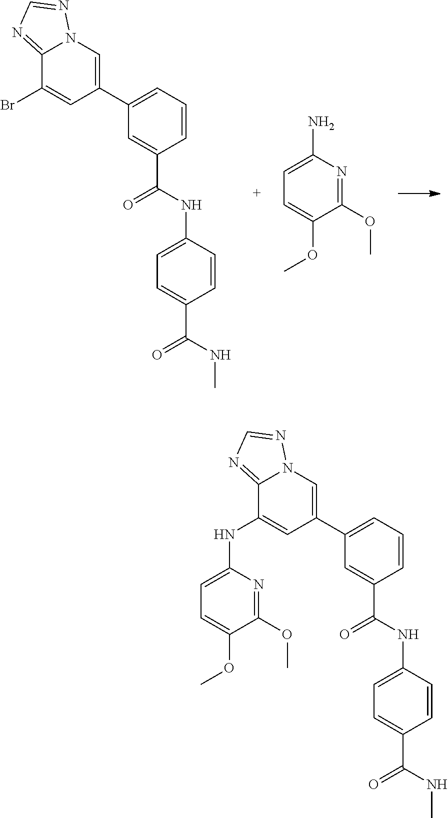

### Procedure:

To a stirred solution of 3-(8-bromo-[1,2,4]triazolo[1,5-a]pyridin-6-yl)-N-(4-(methylcarbamoyl)phenyl)benzamide (180 mg, 0.4 mmol), 5,6-dimethoxypyridin-2-amine (92 mg, 0.59 mmol), X-Phos (100 mg, 0.21 mmol) and Cs₂CO₃ (504 mg, 1.54 mmol) in dioxane (50 mL) was added Pd₂(dba)₃ (52 mg, 0.056 mmol) under nitrogen at room temperature. The reaction mixture was degassed by bubbling nitrogen for 10 minutes and then stirred at 95° C. under nitrogen for 24 h. The solvent was evaporated and the residue was purified by column chromatography (silica gel, 200-300 mesh, methanol in ethyl acetate 1:20, v/v) to give 3-(8-(5,6-dimethoxypyridin-2-ylamino)-[1,2,4]triazolo[1,5-a]pyridin-6-yl)-N-(4-(methylcarbamoyl)phenyl)benzamide (18 mg, 8.5%) as a solid. ¹H NMR (300 MHz, DMSO): δ 10.53 (s, 1H), 9.36 (s, 1H), 8.95 (s, 1H), 8.90 (s, 1H), 8.55 (s, 1H), 8.36-8.36 (m, 2H), 8.03-7.99 (m, 2H), 7.88 (s, 4H), 7.71 (t, 1H, J=7.8 Hz), 7.39 (d, 1H, J=8.4 Hz), 7.01 (d, 1H, J=8.4 Hz), 3.96 (s, 1H), 3.75 (s, 1H), 2.80 (d, 3H, J=4.5 Hz). LC/MS: 524 [M+H]⁺. HPLC: 96.83% at 214 nm, 95.23% at 254 nm, t_{R}=4.28 min.

### Example 6

### (1,4-Diazepan-1-yl)(3-(8-(5,6-dimethoxypyridin-2-ylamino)-[1,2,4]-triazolo[1,5-a]pyridin-6-yl)phenyl)methanone hydrochloride

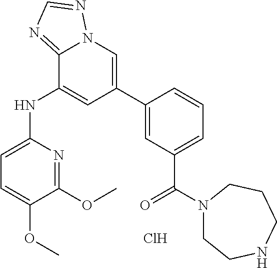

### Step 1

### tert-Butyl 4-(3-(4,4,5,5-tetramethyl-1,3,2-dioxaborolan-2-yl)benzoyl)-1,4-diazepane-1-carboxylate

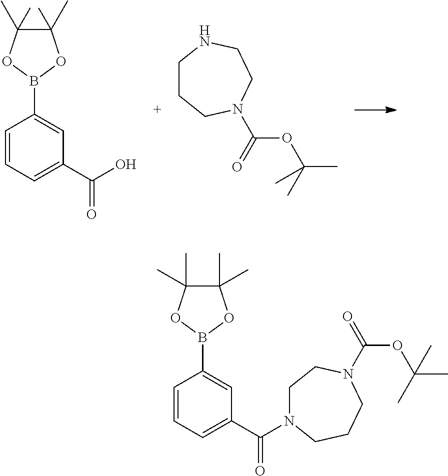

### Procedure:

A solution of 3-(4,4,5,5-tetramethyl-1,3,2-dioxaborolan-2-yl)benzoic acid (0.8 g, 3.2 mmol), tert-butyl 1,4-diazepane-1-carboxylate (0.77 g, 3.8 mmol), HATU (1.47 g, 3.8 mmol) and DIEA (1 g, 7.25 mmol) in DMF (10 mL) was stirred at room temperature for 24 h. The excess solvent was evaporated at 70° C. under reduced pressure then the residue was purified by column chromatography (silica gel, 200-300 mesh, ethyl acetate/petroleum ether 2:4, v/v) to give 4-(3-(4,4,5,5-tetramethyl-1,3,2-dioxaborolan-2-yl)benzoyl)-1,4-diazepane-1-carboxylate (0.73 g, 52.8%) as a white solid. LC/MS: 883.4 [2M+Na]⁺.

### Step 2

### tert-Butyl 4-(3-(8-bromo-[1,2,4]triazolo[1,5-a]pyridin-6-yl)benzoyl)-1,4-diazepane-1-carboxylate

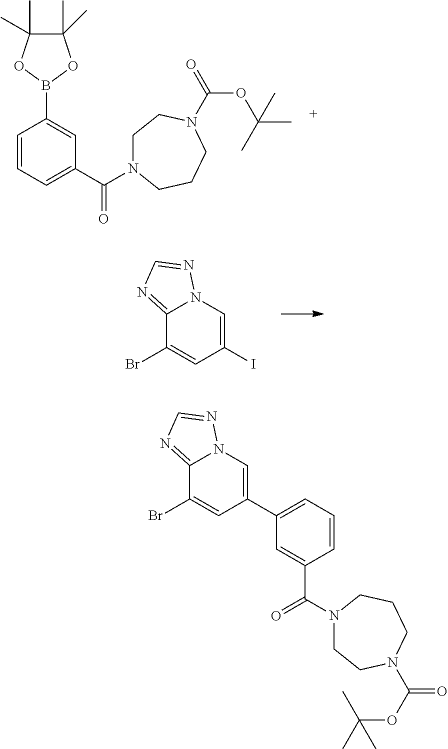

### Procedure:

To a stirred solution of tert-butyl 4-(3-(4,4,5,5-tetramethyl-1,3,2-dioxaborolan-2-yl)benzoyl)-1,4-diazepane-1-carboxylate (0.5 g, 1.16 mmol), 8-bromo-6-iodo-[1,2,4]triazolo[1,5-a]pyridine (0.413 g, 1.27 mmol), Na₂CO₃ (0.479 g, 4.5 mmol) and H₂O (5 mL) in dioxane (50 mL) at room temperature and under nitrogen was added Pd(PPh₃)₄ (0.144 g, 0.11 mmol), then the reaction mixture was degassed by bubbling nitrogen for 10 minutes. The reaction mixture was heated at 95° C. for 24 h then cooled. The solvent was evaporated and the residue filtered through a plug of silica gel. The filter cake was washed with ethyl acetate/petroleum ether (2:1, v/v) and the filtrates combined and concentrated to give crude tert-butyl 4-(3-(8-bromo-[1,2,4]triazolo[1,5-a]pyridin-6-yl)benzoyl)-1,4-diazepane-1-carboxylate (0.365 g, 62.7%) as a white foam. This was used directly in the next step without further purification. LC/MS: 500.0 [M+H]⁺.

### Step 3

### tert-Butyl 4-(3-(8-(5,6-dimethoxypyridin-2-ylamino)-[1,2,4]triazolo[1,5-a]pyridin-6-yl)benzoyl)-1,4-diazepane-1-carboxylate

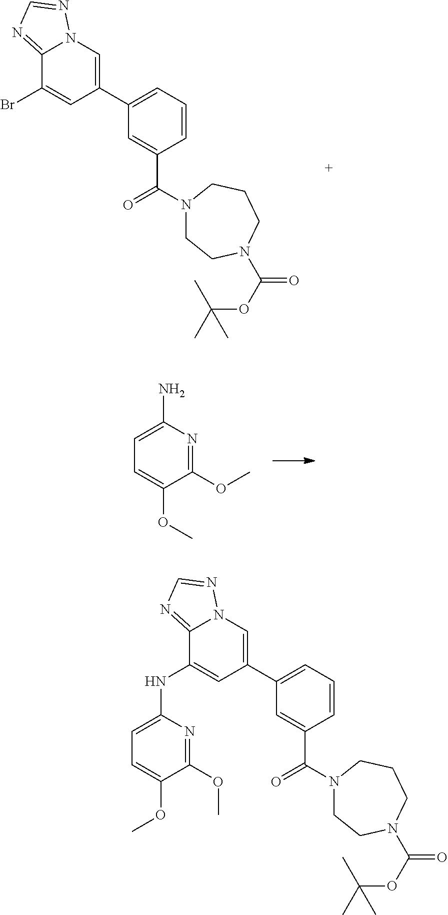

### Procedure:

A mixture of tert-butyl 4-(3-(8-bromo-[1,2,4]triazolo[1,5-a]pyridin-6-yl)benzoyl)-1,4-diazepane-1-carboxylate (365 mg, 0.73 mmol), 5,6-dimethoxypyridin-2-amine (169 mg, 1.09 mmol), X-Phos (182 mg, 0.38 mmol), Pd₂(dba)₃ (94 mg, 0.10 mmol) and Cs₂CO₃ (917 mg, 2.8 mmol) in dioxane (50 mL) was degassed by bubbling nitrogen for 10 minutes. The reaction mixture was heated at 95° C. under nitrogen for 24 h. The solvent was evaporated and the residue was filtered through a plug of silica gel. The filter cake was washed with ethyl acetate/petroleum ether (2:1, v/v) and the combined filtrates concentrated to give crude tert-butyl 4-(3-(8-(5,6-dimethoxypyridin-2-ylamino)-[1,2,4]triazolo[1,5-a]pyridin-6-yl)benzoyl)-1,4-diazepane-1-carboxylate (160 mg, 38.2%) as a solid. This was used directly without further purification. LC/MS: 574.3 [M+H]⁺.

### Step 4

### (1,4-Diazepan-1-yl)(3-(8-(5,6-dimethoxypyridin-2-ylamino)-[1,2,4]triazolo[1,5-a]pyridin-6-yl)phenyl)methanone hydrochloride

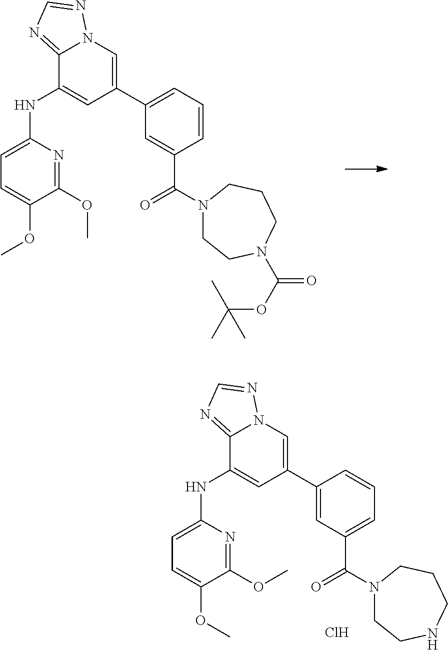

### Procedure:

To a stirred solution of tert-butyl 4-(3-(8-(5,6-dimethoxypyridin-2-ylamino)-[1,2,4]triazolo[1,5-a]pyridin-6-yl)benzoyl)-1,4-diazepane-1-carboxylate (155 mg, 0.27 mmol) in dichloromethane (30 mL) was added trifluoroacetic acid (5 mL) at room temperature. After 24 h the excess solvent was evaporated and the residue was purified by preparative-HPLC (Gemini 5u C18 150×21.2 mm; inject volume: 3 ml/inj, flow rate: 20 mL/min; wavelength: 214 nm and 254 nm; gradient conditions: 20% acetonitrile/80% water (0.1% TFA, v/v) proceeding to 40% acetonitrile/60% water (0.1% TFA, v/v) in a linear fashion after 9 min.) to give a solid residue. This was dissolved in 5 mL of methanol and 1 mL of water the acidified with 1N HCl to pH=1. The solvent was evaporated at 40° C. at reduced pressure to give (1,4-diazepan-1-yl)(3-(8-(5,6-dimethoxypyridin-2-ylamino)-[1,2,4]triazolo[1,5-a]pyridin-6-yl)phenyl)methanone hydrochloride (40 mg, 29%) as a solid. ¹H NMR (300 MHz, DMSO): δ 9.34 (s, 1H), 9.08 (brs, 2H), 8.92 (s, 1H), 8.79 (s, 1H), 8.54 (s, 1H), 7.88-7.84 (m, 2H), 7.59 (t, 1H, J=7.6 Hz), 7.50 (d, 1H, J=7.5 Hz), 7.39 (d, 1H, J=8.4 Hz), 7.00 (d, 1H, J=8.4 Hz), 3.96 (s, 3H), 3.88 (brs, 2H), 3.75 (s, 3H), 3.57 (brs, 2H), 3.47-3.18 (m, 4H), 1.99 (brs, 2H). LC/MS: 474 [M+H]⁺. HPLC: 99.86% at 214 nm, 99.85% at 254 nm, t_{R}=4.51 min.

### Example 7

### 3-(8-(5,6-Dimethoxypyridin-2-ylamino)-[1,2,4]triazolo[1,5-a]pyridin-6-yl)-N-(2-oxoindolin-5-yl)benzamide

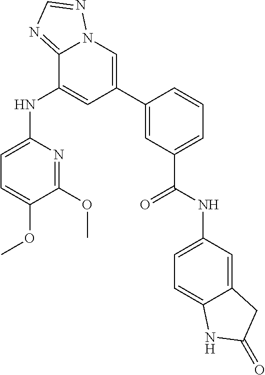

### Step 1

### 3-Bromo-5-iodopyridin-2-amine

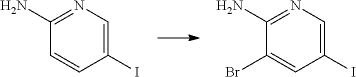

### Procedure:

To a stirred solution of 5-iodopyridin-2-amine (25.0 g, 113 mmol) in acetonitrile (500 mL) was added NBS (20.2 g, 113 mmol) slowly at room temperature. After the addition, the reaction mixture was stirred at room temperature for a further 72 h. The solvent was evaporated at 40° C. at reduced pressure and the residue was purified by column chromatography (silica gel, 200-300 mesh, ethyl acetate/petroleum ether 3:1, v/v) to give 3-bromo-5-iodopyridin-2-amine (15.9 g, 47%) as a yellow solid. ¹H NMR (300 MHz, DMSO): δ 8.07 (s, 1H), 7.98-7.97 (m, 1H), 6.43 (brs, 1H). LC/MS: 298.9 [M+H]⁺.

### Step 2

### (E)-N′-(3-Bromo-5-iodopyridin-2-yl)-N-hydroxyformimidamide

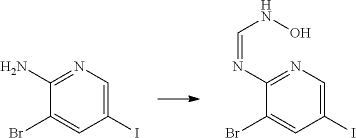

### Procedure:

To a stirred solution of 3-bromo-5-iodopyridin-2-amine (2 g, 6.69 mmol) in 40 mL of isopropanol under nitrogen was added N,N-dimethylformamide dimethyl acetal (1.3 g, 10.9 mmol) in one portion, then the mixture was stirred at reflux for 5 h. The reaction was cooled to 50° C., and sodium bicarbonate (1.5 g, 17.8 mmol), hydroxylamine hydrochloride (1 g, 14.5 mmol) were added successively and slowly. Then the reaction mixture was stirred 50° C. for 16 h. The solvent was evaporated at 40° C. under reduced pressure and the residue was purified by column chromatography (silica gel, 200-300 mesh, dichloromethane/methanol 100:5, v/v) to give (E)-N′-(3-bromo-5-iodopyridin-2-yl)-N-hydroxyformimidamide (2.2 g, 96%) as an off-white solid. ¹H NMR (300 MHz, DMSO+D₂O): δ 8.38-8.31 (m, 2H), 7.85 (d, 1H, J=2.1 Hz). LC/MS: 341.9 [M+H]⁺.

### Step 3

### 8-Bromo-6-iodo-[1,2,4]-triazolo[1,5-a]pyridine

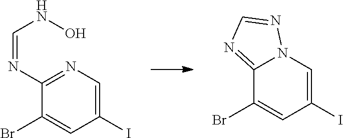

### Procedure:

To a stirred solution of (E)-N-(3-bromo-5-iodopyridin-2-yl)-N-hydroxyformimidamide (4.2 g, 12.2 mmol) in THF (120 mL) at 0° C. was added dropwise 2,2,2-trifluoroacetic anhydride (10 mmol) slowly. After the addition, the reaction mixture was stirred at room temperature for 24 h. Then the reaction mixture was treated by saturated sodium bicarbonate solution until it reached pH=8. The solvent was evaporated at 40° C. at reduced pressure to give a crude product. This was purified by column chromatography (silica gel, 200-300 mesh, dichloromethane and methanol 100:1.5, v/v) to give 8-bromo-6-iodo-[1,2,4]triazolo[1,5-a]pyridine (2.45 g, 62%) as a pale yellow solid. ¹H NMR (300 MHz, CD₃OD): δ 9.16 (d, 1H, J=1.2 Hz), 8.38 (s, 1H), 8.20 (d, 1H, J=0.9 Hz). LC/MS: 323.8 [M+H]⁺.

### Step 4

### Methyl 3-(8-bromo-[1,2,4]-triazolo[1,5-a]pyridin-6-yl)benzoate

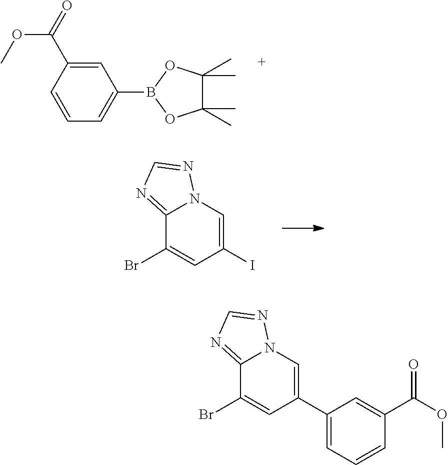

### Procedure:

To a stirred solution of 8-bromo-6-iodo-[1,2,4]triazolo[1,5-a]pyridine (0.945 g, 2.9 mmol), methyl 3-(4,4,5,5-tetramethyl-1,3,2-dioxaborolan-2-yl)benzoate (0.72 g, 2.74 mmol) and sodium carbonate (0.9 g, 8.4 mmol) in water (9 mL) and dioxane (90 mL) at room temperature under nitrogen was added Pd(PPh₃)₄ (0.168 g, 0.14 mmol) in one portion. Then the reaction mixture was degassed by bubbling nitrogen for 15 minutes. The final mixture was heated at reflux for 24 h under nitrogen. The solvent was evaporated at 40° C. at reduced pressure and the residue was purified by column chromatography (silica gel, 200-300 mesh, ethyl acetate/petroleum ether 1:2, v/v) to give methyl 3-(8-bromo-[1,2,4]triazolo[1,5-a]pyridin-6-yl)benzoate (0.644 g, 66%) as a white solid. ¹H NMR (300 MHz, DMSO): δ 9.45 (s, 1H), 8.62 (s, 1H), 8.44-8.42 (m, 1H), 8.32 (s, 1H), 8.12 (s, 1H), 8.02-8.00 (m, 1H), 7.68-7.57 (m, 1H), 3.95 (s, 1H). LC/MS: 331.9 [M+H]⁺.

### Step 5

### Methyl 3-(8-(5,6-dimethoxypyridin-2-ylamino)-[1,2,4]-triazolo[1,5-a]pyridin-6-yl)benzoate

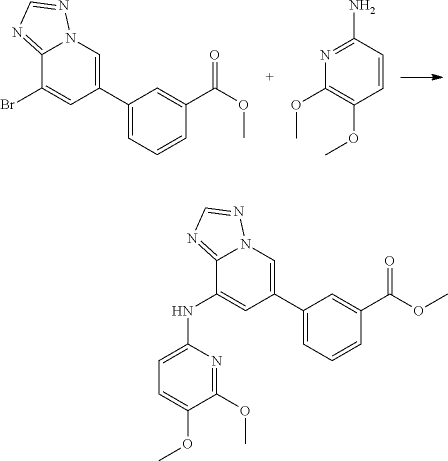

### Procedure:

To a stirred solution of methyl 3-(8-bromo-[1,2,4]triazolo[1,5-a]pyridin-6-yl)benzoate (0.644 g, 1.94 mmol), 5,6-dimethoxypyridin-2-amine (0.358 g, 2.23 mmol), X-Phos (0.452 g, 0.95 mmol) and Cs₂CO₃ (2.2 g, 6.79 mmol) in dry dioxane (50 mL) at room temperature under nitrogen was added Pd₂(dba)₃ (0.211 g, 0.36 mmol) in one portion. After the addition, the reaction mixture was degassed by bubbling nitrogen for 10 minutes and then stirred at 96° C. under nitrogen for 24 h. The solvent was evaporated at 40° C. at reduced pressure and the residue was filtered through a plug of silica gel with ethyl acetate/petroleum ether 1:2, v/v) to give methyl 3-(8-(5,6-dimethoxypyridin-2-ylamino)-[1,2,4]triazolo[1,5-a]pyridin-6-yl)benzoate (0.528 g, 67%) as a solid containing additional unidentified impurities. This was used directly without further purification. LC/MS: 405.9 [M+H]⁺.

### Step 6

### Potassium 4-(8-(5,6-dimethoxypyridin-2-ylamino)-[1,2,4]-triazolo[1,5-a]pyridin-6-yl)benzoate

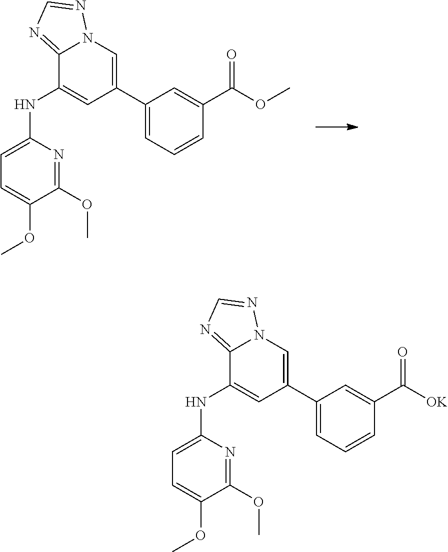

### Procedure:

To a stirred solution of methyl 4-(8-(5,6-dimethoxypyridin-2-ylamino)-[1,2,4]triazolo[1,5-a]pyridin-6-yl)benzoate (0.29 g, 0.71 mmol) in methanol (2 mL) and THF (30 mL) was added a solution of KOH (0.198 g, 3.53 mmol) in water (2 mL) at room temperature. Then the reaction mixture was stirred at room temperature for 16 h. The solvent was evaporated at 40° C. at reduced pressure to give potassium 4-(8-(5,6-dimethoxypyridin-2-ylamino)-[1,2,4]triazolo[1,5-a]pyridin-6-yl)benzoate (505 mg, crude) as a solid, containing an excess of KOH. This was used directly in the next step without further purification. LC/MS: 392.1 [M+H]⁺.

### Step 7

### 3-(8-(5,6-Dimethoxypyridin-2-ylamino)-[1,2,4]-triazolo[1,5-a]pyridin-6-yl)-N-(2-oxoindolin-5-yl)benzamide

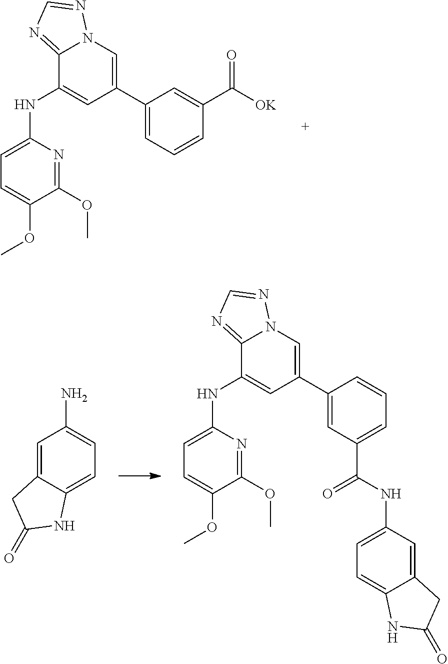

### Procedure:

To a stirred solution of potassium 3-(8-(5,6-dimethoxypyridin-2-ylamino)-[1,2,4]triazolo[1,5-a]pyridin-6-yl)benzoate (0.505 g, 0.71 mmol), 5-aminoindolin-2-one (0.138 g, 0.93 mmol) and HATU (0.353 g, 0.93 mmol) in DMF (12 mL) was added hydrochloride salt of DIEA (0.646 g, 3.9 mmol) in one portion at room temperature. After 16 h the solvent was evaporated at 70° C. at reduced pressure and the residue was purified by column chromatography (silica gel, 200-300 mesh, methanol and ethyl acetate 5:100, v/v) to give 3-(8-(5,6-dimethoxypyridin-2-ylamino)-[1,2,4]triazolo[1,5-a]pyridin-6-yl)-N-(2-oxoindolin-5-yl)benzamide (0.28 g, 75%) as a solid. This was further purified by preparative-HPLC (Gemini 5u C18 150×21.2 mm; inject volume: 3 ml/inj, flow rate: 20 mL/min; wavelength: 214 nm and 254 nm; gradient conditions: 40% acetonitrile/60% water (0.1% TFA, v/v) initially, proceeding to 60% acetonitrile/40% water (0.1% TFA, v/v) in a linear fashion after 9 min.) to give 3-(8-(5,6-dimethoxypyridin-2-ylamino)-[1,2,4]triazolo[1,5-a]pyridin-6-yl)-N-(2-oxoindolin-5-yl)benzamide 2,2,2-trifluoroacetate (6 mg. 0.97%) as a pale yellow solid. ¹H NMR (300 MHz, DMSO): δ 10.35 (s, 1H), 10.22 (s, 1H), 9.34 (s, 1H), 8.96 (s, 1H), 8.88 (s, 1H), 8.54 (s, 1H), 8.33 (s, 1H), 7.98-7.96 (m, 2H), 7.68-7.51 (m, 3H), 7.38 (d, 1H, J=8.4 Hz), 7.00 (d, 1H, J=8.1 Hz), 6.82 (d, 1H, J=8.4 Hz), 3.96 (s, 3H), 3.74 (s, 3H), 3.52 (s, 2H). LC/MS: 522 [M+H]⁺. HPLC: 99.16% at 214 nm, 100% at 254 nm, t_{R}=5.75 min.

### Example 8

### 4-(8-(5,6-Dimethoxypyridin-2-ylamino)-[1,2,4]triazolo[1,5-a]pyridin-6-yl)-N-(2-(pyridin-4-yl)ethyl)benzamide

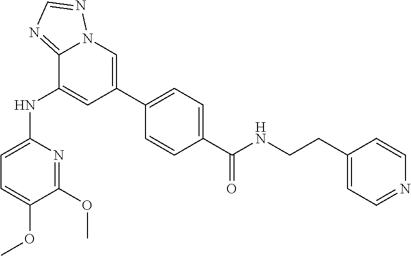

### Step 1

### N-(2-(Pyridin-4-yl)ethyl)-4-(4,4,5,5-tetramethyl-1,3,2-dioxaborolan-2-yl)benzamide

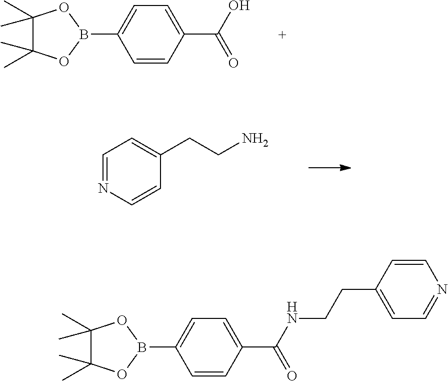

### Procedure:

A solution of 4-(4,4,5,5-tetramethyl-1,3,2-dioxaborolan-2-yl)benzoic acid (0.880 g, 3.54 mmol), 2-(pyridin-4-yl)ethanamine (0.517 g, 4.23 mmol), HATU (1.6 g, 4.23 mmol) and DIEA (1.1 g, 8.52 mmol) in DMF (30 mL) was stirred at room temperature for 16 h. The excess solvent was evaporated at 70° C. at reduced pressure and the residue was filtered through a plug of silica gel. The filter cake was washed with methanol/ethyl acetate (5:100, v/v) and the combined filtrates evaporated to give N-(2-(pyridin-4-yl)ethyl)-4-(4,4,5,5-tetramethyl-1,3,2-dioxaborolan-2-yl)benzamide (1.08 g, 86%). This was used directly without further purification. LC/MS: 353.0 [M+H]⁺.

### Step 2

### 4-(8-Bromo-[1,2,4]-triazolo[1,5-a]pyridin-6-yl)-N-(2-(pyridin-4-yl)ethyl)benzamide

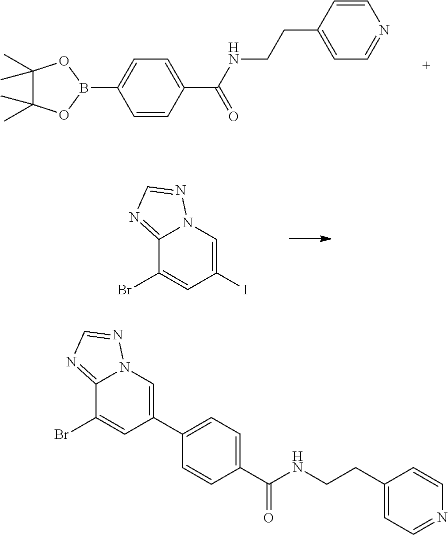

### Procedure:

To a solution of N-(2-(pyridin-4-yl)ethyl)-4-(4,4,5,5-tetramethyl-1,3,2-dioxaborolan-2-yl)benzamide (0.352 g, 1 mmol), 8-bromo-6-iodo-[1,2,4]triazolo[1,5-a]pyridine (0.323 g, 1 mmol), sodium carbonate (0.4126 g, 3.8 mmol) in water (4 mL) and dioxane (50 mL) was added Pd(PPh₃)₄ (0.124 g, 0.1 mmol) in one portion under nitrogen at room temperature. Then the reaction mixture was degassed by bubbling nitrogen for 10 minutes and then heated at 96° C. under nitrogen for 16 h. The solvent was evaporated at 40° C. at reduced pressure and the residue was filtered through a plug of silica gel. The filter cake was washed with methanol/ethyl acetate 5:100, v/v) and the combined filtrates concentrated to give crude 4-(8-bromo-[1,2,4]triazolo[1,5-a]pyridin-6-yl)-N-(2-(pyridin-4-yl)ethyl)benzamide (0.255 g, 60%) as a white solid. This was used directly without further purification. LC/MS: 422.0 [M+H]⁺.

### Step 3

### 4-(8-(5,6-Dimethoxypyridin-2-ylamino)-[1,2,4]triazolo[1,5-a]pyridin-6-yl)-N-(2-(pyridin-4-yl)ethyl)benzamide

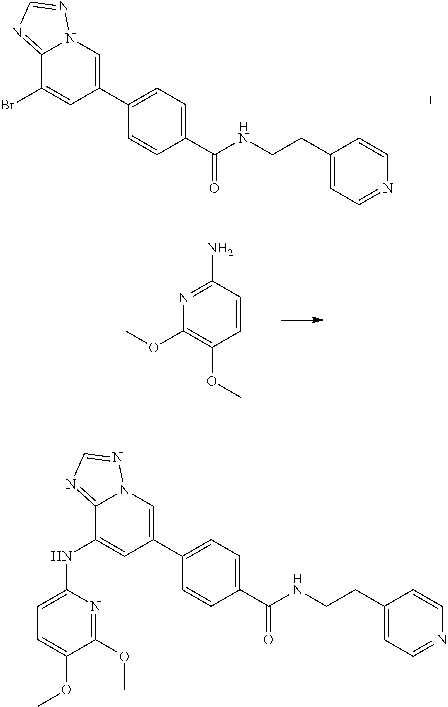

### Procedure:

To a solution of 4-(8-bromo-[1,2,4]triazolo[1,5-a]pyridin-6-yl)-N-(2-(pyridin-4-yl)ethyl)benzamide (0.255 g, 0.6 mmol), 5,6-dimethoxypyridin-2-amine (0.186 g, 1.2 mmol), X-Phos (0.182 g, 0.38 mmol) and Cs₂CO₃ (0.917 g, 2.8 mmol) in 50 mL of dioxane was added Pd₂(dba)₃ (0.094 g, 0.1 mmol) in one portion under nitrogen at room temperature. Then the reaction mixture was degassed by bubbling nitrogen for 10 minutes and then heated at 96° C. under nitrogen for 16 h. The solvent was evaporated at 40° C. at reduced pressure and the residue was purified by column chromatography (silica gel, 200-300 mesh, methanol/ethyl acetate 10:100, v/v) to give 4-(8-(5,6-dimethoxypyridin-2-ylamino)-[1,2,4]triazolo[1,5-a]pyridin-6-yl)-N-(2-(pyridin-4-yl)ethyl)benzamide (0.085 g, 28%) as a pale yellow solid. ¹H NMR (300 MHz, DMSO): δ 9.31 (s, 1H), 8.87 (s, 1H), 8.79 (s, 1H), 8.65 (t, 1H, J=5.4 Hz), 8.50-8.44 (m, 3H), 7.92-7.82 (m, 4H), 7.37-7.26 (m, 3H), 6.97 (d, 1H, J=8.4 Hz), 3.95 (s, 3H), 3.74 (s, 3H), 3.59-3.52 (m, 2H), 2.89 (t, 2H, J=6.9 Hz). LC/MS: 496 [M+H]⁺. HPLC: 95.04% at 214 nm, 97.77% at 254 nm, t_{R}=4.86 min.

### Example 9

### 3-(8-(5,6-Dimethoxypyridin-2-ylamino)-[1,2,4]-triazolo[1,5-a]pyridin-6-yl)-N-(1H-indazol-5-yl)benzamide

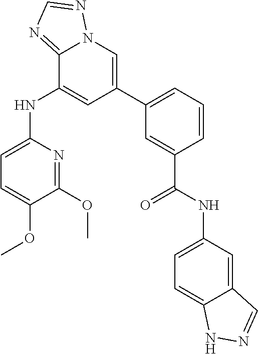

### Step 1

### N-(1H-Indazol-5-yl)-3-(4,4,5,5-tetramethyl-1,3,2-dioxaborolan-2-yl)benzamide

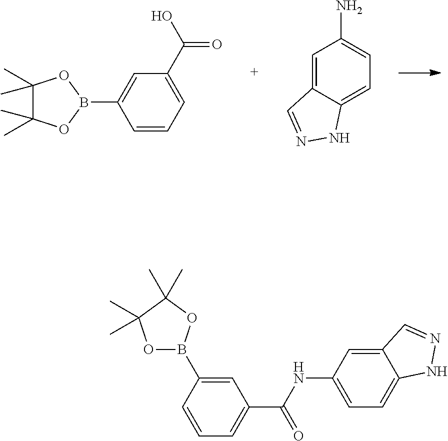

### Procedure:

To a stirred solution of 3-(4,4,5,5-tetramethyl-1,3,2-dioxaborolan-2-yl)benzoic acid (0.516 g, 2.08 mmol) in DMF (10 mL) were added 1H-indazol-5-amine (0.332 g, 2.49 mmol), HATU (0.946 g, 2.49 mmol) and DIEA (0.67 g, 5.19 mmol) in one portion at room temperature. After 24 h the solvents were evaporated at 70° C. at reduced pressure and the residue purified by column chromatography (silica gel, 200-300 mesh, petroleum ether/ethyl acetate 2:4, v/v) to give N-(1H-indazol-5-yl)-3-(4,4,5,5-tetramethyl-1,3,2-dioxaborolan-2-yl)benzamide (0.105 g, 13.9%). ¹H NMR (300 MHz, DMSO): δ 13.01 (s, 1H), 10.36 (s, 1H), 8.72-8.70 (m, 1H), 8.52-8.48 (m, 1H), 8.25-8.23 (m, 1H), 8.07-8.05 (m, 1H), 7.65-7.46 (m, 1H), 1.32 (s, 12H). LC/MS: 364.0 [M+H]⁺.

### Step 2

### 3-(8-(5,6-Dimethoxypyridin-2-ylamino)-[1,2,4]triazolo[1,5-a]pyridin-6-yl)-N-(1H-indazol-5-yl)benzamide

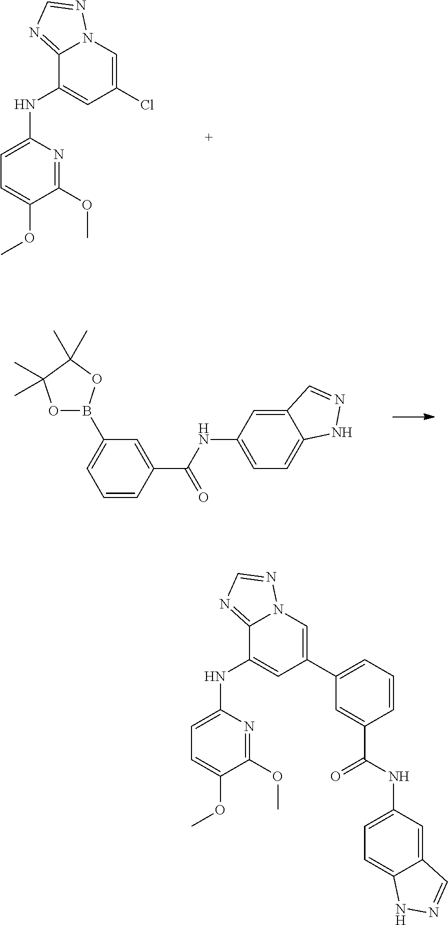

### Procedure:

To a solution of 6-chloro-N-(5,6-dimethoxypyridin-2-yl)-[1,2,4]triazolo[1,5-a]pyridin-8-amine (0.06 g, 0.196 mmol), N-(1H-indazol-5-yl)-3-(4,4,5,5-tetramethyl-1,3,2-dioxaborolan-2-yl)benzamide (0.100 g, 0.275 mmol), sodium carbonate (0.100 g, 0.94 mmol), CsF (0.029 g, 0.191 mmol) in water (0.5 mL) and DMF (6 mL) was added Pd(PPh₃)₄ (0.025 g, 0.02 mmol) in one portion under nitrogen at room temperature. Then the reaction mixture was degassed by bubbling nitrogen for 10 minutes and then heated at 96° C. under nitrogen for 16 h. The solvent was evaporated at 70° C. at reduced pressure and the residue was purified by column chromatography (silica gel, 200-300 mesh, methanol/dichloromethane 1:50, v/v) to give 3-(8-(5,6-dimethoxypyridin-2-ylamino)-[1,2,4]triazolo[1,5-a]pyridin-6-yl)-N-(1H-indazol-5-yl)benzamide (0.013 g, 13%) as an off-white solid. ¹H NMR (300 MHz, DMSO): δ 13.04 (s, 1H), 10.37 (s, 1H), 9.35 (s, 1H), 8.97 (s, 1H), 8.90 (s, 1H), 8.54 (s, 1H), 8.37 (s, 1H), 8.26 (s, 1H), 8.08-8.01 (m, 3H), 7.69-7.54 (m, 3H), 7.38 (d, 1H, J=8.4 Hz), 7.01 (d, 1H, J=8.4 Hz), 3.97 (s, 3H), 3.74 (s, 3H). LC/MS: 507 [M+H]⁺. HPLC: 99.52% at 214 nm, 99.21% at 254 nm, t_{R}=5.99 min.

### Example 10

### 4-(3-(8-(6-(2-Methylpyrrolidin-1-yl)pyridin-2-ylamino)-[1,2,4]triazolo[1,5-a]pyridin-6-yl)benzamido)benzoic acid hydrochloride

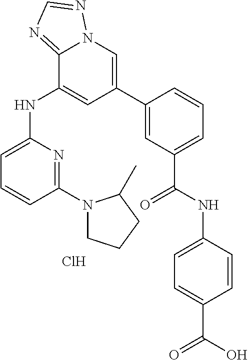

### Step 1

### tert-Butyl 4-(3-(4,4,5,5-tetramethyl-1,3,2-dioxaborolan-2-yl)benzamido)benzoate

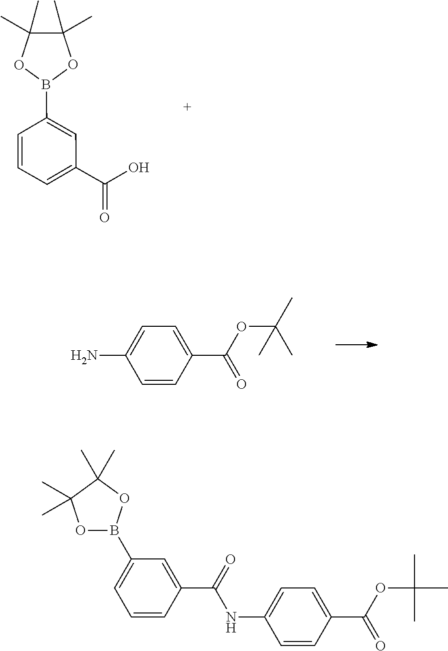

### Procedure:

A solution of 3-(4,4,5,5-tetramethyl-1,3,2-dioxaborolan-2-yl)benzoic acid (0.6 g, 2.4 mmol), tert-butyl 4-aminobenzoate (0.56 g, 2.9 mmol), HATU (1.1 g, 2.9 mmol) and DIEA (0.75 g, 5.8 mmol) in DMF (7 mL) was stirred at room temperature for 16 h. The solvent was evaporated at 70° C. at reduced pressure and the residue was filtered through a plug of silica gel. The filter cake was washed with ethyl acetate/petroleum ether (1:8, v/v) and the combined filtrates concentrated to give crude tert-butyl 4-(3-(4,4,5,5-tetramethyl-1,3,2-dioxaborolan-2-yl)benzamido)benzoate (1.0 g, 97%) as a colourless oil. This was used directly without further purification. LC/MS: 424.2 [M+H]⁺.

### Step 2

### tert-Butyl 4-(3-(8-bromo-[1,2,4]triazolo[1,5-a]pyridin-6-yl)benzamido)benzoate

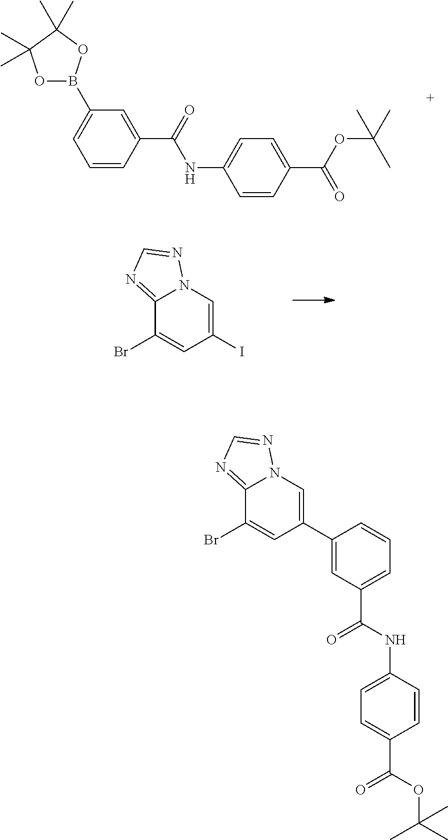

### Procedure:

To a solution of tert-butyl 4-(3-(4,4,5,5-tetramethyl-1,3,2-dioxaborolan-2-yl)benzamido)benzoate (0.3 g, 0.7 mmol), 8-bromo-6-iodo-[1,2,4]triazolo[1,5-a]pyridine (0.23 g, 0.7 mmol), sodium carbonate (0.255 g, 2.4 mmol) in dioxane (50 mL) and water (4 mL) was added Pd(PPh₃)₄ (0.08 g, 0.06 mmol) in one portion at room temperature under nitrogen. Then the reaction mixture was degassed by bubbling nitrogen for 10 minutes and then stirred at 96° C. for 16 h under nitrogen. The solvent was evaporated at 40° C. at reduced pressure and the residue was filtered through a plug of silica gel. The filter cake was washed with ethyl acetate/petroleum ether (1:1, v/v) and the combined filtrates were concentrated to give crude tert-butyl 4-(3-(8-bromo-[1,2,4]triazolo[1,5-a]pyridin-6-yl)benzamido)benzoate (0.150 g, 43%) as a pale yellow solid. LC/MS: 493.0 [M+H]⁺.

### Step 3

### 6-(2-Methylpyrrolidin-1-yl)pyridin-2-amine

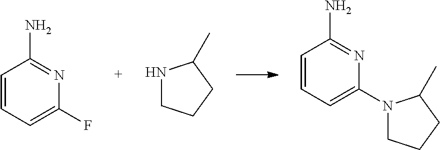

### Procedure:

To a stirred solution of 6-fluoropyridin-2-amine (0.224 g, 1 mmol) and 2-methylpyrrolidine (0.173 g, 2 mmol) in DMSO (10 mL) were added KF (0.116 g, 2 mmol), potassium carbonate (0.276 g, 2 mmol) and copper acetate (0.199 g, 1 mmol) successively at room temperature. Then the mixture was heated at 150° C. for 4 h. The reaction was cooled and poured into water (20 mL) then extracted with ethyl acetate (60 mL). The organic phase was dried over anhydrous sodium sulphate. The solvent was evaporated and the residue was filtered through a plug of silica gel. The filter cake was washed with ethyl acetate/petroleum ether (1:10, v/v) and the combined filtrates concentrated to give crude 6-(2-methylpyrrolidin-1-yl)pyridin-2-amine (0.225 g, 64%) as a colourless oil. This was used directly without further purification. LC/MS: 178.1 [M+H]⁺.

### Step 4

### tert-Butyl 4-(3-(8-(6-(2-methylpyrrolidin-1-yl)pyridin-2-ylamino)-[1,2,4]triazolo[1,5-a]pyridin-6-yl)benzamido)benzoate

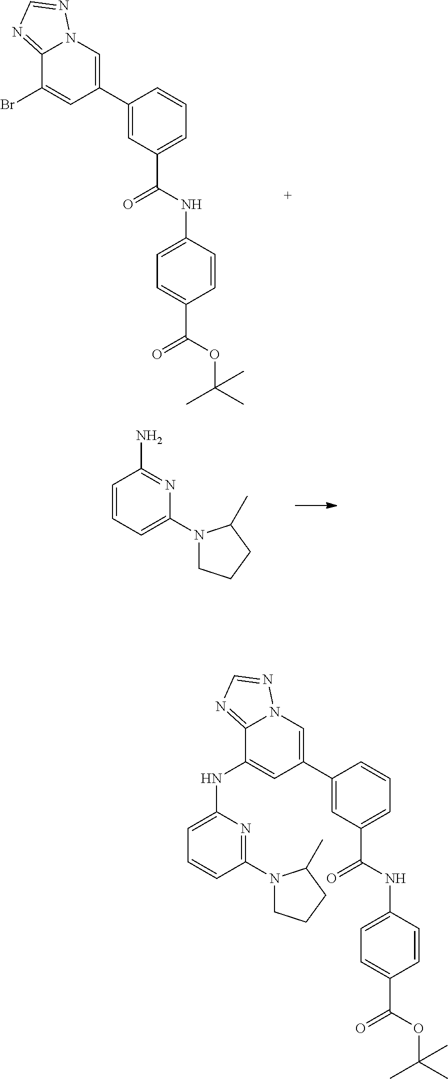

### Procedure:

To a stirred solution of tert-butyl 4-(3-(8-bromo-[1,2,4]triazolo[1,5-a]pyridin-6-yl)benzamido)benzoate (0.143 g, 0.29 mmol), 6-(2-methylpyrrolidin-1-yl)pyridin-2-amine (0.077 g, 0.43 mmol), BINAP (0.072 g, 0.11 mmol) and Cs₂CO₃ (0.283 g, 0.86 mmol) in dioxane (15 mL) was added Pd₂(dba)₃ (0.026 g, 0.028 mmol) in one portion at room temperature under nitrogen. The mixture was degassed by bubbling nitrogen for 10 minutes and then stirred at reflux under nitrogen for 16 h. The solvent was evaporated and the residue was filtered through a plug of silica gel. The filter cake was washed with ethyl acetate/petroleum ether (1:1, v/v), and the combined filtrates concentrated to give crude tert-butyl 4-(3-(8-(6-(2-methylpyrrolidin-1-yl)pyridin-2-ylamino)-[1,2,4]triazolo[1,5-a]pyridin-6-yl)benzamido)benzoate (0.110 g, 64%) as a pale yellow solid. This was used directly without further purification. LC/MS: 590.3 [M+H]⁺. LC/MS: 590.3 [M+H]⁺.

### Step 5

### 4-(3-(8-(6-(2-Methylpyrrolidin-1-yl)pyridin-2-ylamino)-[1,2,4]triazolo[1,5-a]pyridin-6-yl)benzamido)benzoic acid hydrochloride

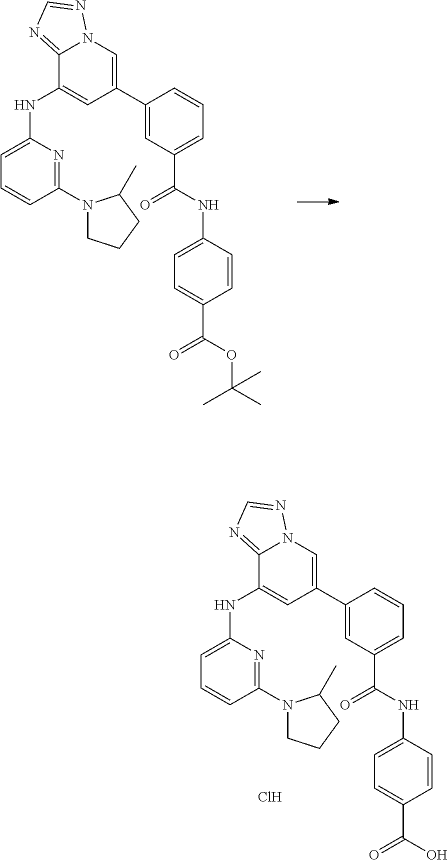

### Procedure:

To a stirred solution of tert-butyl 4-(3-(8-(6-(2-methylpyrrolidin-1-yl)pyridin-2-ylamino)-[1,2,4]triazolo[1,5-a]pyridin-6-yl)benzamido)benzoate (0.110 g, 0.186 mmol) in dichloromethane (10 mL) was added TFA (5 mL) at room temperature. After 16 h the solvent was evaporated at 40° C. at reduced pressure and the residue was purified by preparative-HPLC (Gemini 5u C18 150×21.2 mm; inject volume: 3 ml/inj, flow rate: 20 mL/min; wavelength: 214 nm and 254 nm; gradient conditions: 50% acetonitrile/50% water, 0.1% TFA, v/v) initially, proceeding to 80% acetonitrile/20% water (0.1% TFA, v/v) in a linear fashion after 9 min.). The solution of the purified compound was treated with conc. HCl to pH=1 and the solvents evaporated to give 4-(3-(8-(6-(2-methylpyrrolidin-1-yl)pyridin-2-ylamino)-[1,2,4]triazolo[1,5-a]pyridin-6-yl)benzamido)benzoic acid hydrochloride (8 mg, 7.5%) as a solid. ¹H NMR (300 MHz, DMSO): δ 10.35 (s, 1H), 10.22 (s, 1H), 9.34 (s, 1H), 8.96 (s, 1H), 8.88 (s, 1H), 8.54 (s, 1H), 8.33 (s, 1H), 7.98-7.96 (m, 2H), 7.68-7.51 (m, 3H), 7.38 (d, 1H, J=8.4 Hz), 7.00 (d, 1H, J=8.1 Hz), 6.82 (d, 1H, J=8.4 Hz), 3.96 (s, 3H), 3.74 (s, 3H), 3.52 (s, 2H). LC/MS: 522 [M+H]⁺. HPLC: 99.16% at 214 nm, 100% at 254 nm, t_{R}=5.75 min.

### Example 11

### Methyl 4-(8-(6-(2-methylpyrrolidin-1-yl)pyridin-2-ylamino)-[1,2,4]triazolo[1,5-a]pyridin-6-yl)benzoate

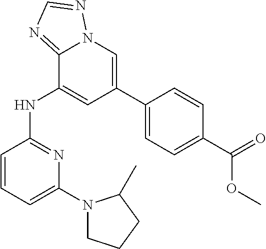

### Step 1

### Methyl 4-(8-bromo-[1,2,4]-triazolo[1,5-a]pyridin-6-yl)benzoate

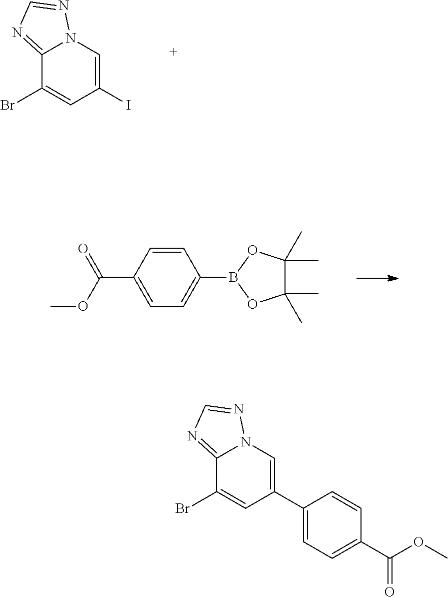

### Procedure:

To a stirred solution of 8-bromo-6-iodo-[1,2,4]triazolo[1,5-a]pyridine (1.29 g, 3.98 mmol), methyl 4-(4,4,5,5-tetramethyl-1,3,2-dioxaborolan-2-yl)benzoate (1.05 g, 4 mmol) and sodium carbonate (1.53 g, 14.4 mmol) in water (10 mL) and dioxane (150 mL) at room temperature under nitrogen was added Pd(PPh₃)₄ (0.46 g, 0.37 mmol) in one portion. Then the reaction mixture was degassed by bubbling nitrogen for six minutes and then stirred at 96° C. for 24 h. The solvent was evaporated at 40° C. at reduced pressure and the residue was filtered through a plug of silica gel. The filter cake was washed with ethyl acetate/petroleum ether (1:2, v/v) and the combined filtrates concentrated to give crude methyl 4-(8-bromo-[1,2,4]triazolo[1,5-a]pyridin-6-yl)benzoate (0.818 g, 61.9%) as a solid. This was used directly without further purification. LC/MS: 331.8 [M+H]⁺.

### Step 2

### Methyl 4-(8-(6-(2-methylpyrrolidin-1-yl)pyridin-2-ylamino)-[1,2,4]triazolo[1,5-a]pyridin-6-yl)benzoate

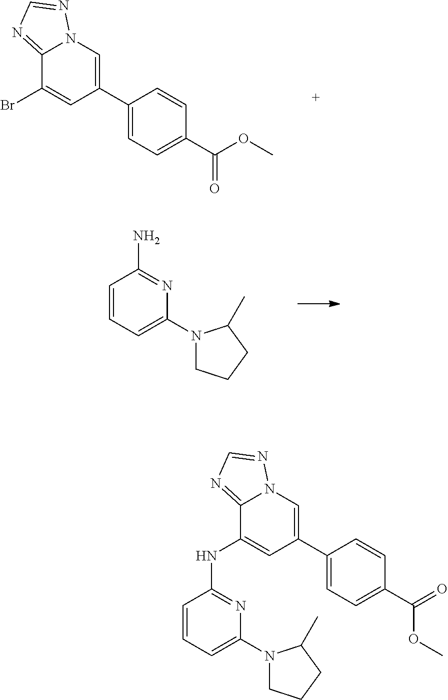

### Procedure:

To a stirred solution of methyl 4-(8-bromo-[1,2,4]triazolo[1,5-a]pyridin-6-yl)benzoate (0.100 g, 0.30 mmol), 6-(2-methylpyrrolidin-1-yl)pyridin-2-amine (0.080 g, 0.45 mmol), BINAP (0.072 g, 0.11 mmol) and Cs₂CO₃ (0.283 g, 0.86 mmol) in dioxane (10 mL) was added Pd₂(dba)₃ (0.026 g, 0.028 mmol) in one portion at room temperature under nitrogen. Then the mixture was degassed by bubbling nitrogen for 10 minutes and then stirred at reflux under nitrogen for 16 h. The solvent was evaporated and the residue was purified by column chromatography (silica gel, 200-300 mesh, ethyl acetate/petroleum ether 1:1, v/v) to give methyl 4-(8-(6-(2-methylpyrrolidin-1-yl)pyridin-2-ylamino)-[1,2,4]triazolo[1,5-a]pyridin-6-yl)benzoate with unidentified impurities. This was further purified by preparative-HPLC (Gemini 5u C18 150×21.2 mm; inject volume: 3 ml/inj, flow rate: 20 mL/min; wavelength: 214 nm and 254 nm; gradient conditions: 35% acetonitrile/65% water (0.1% TFA, v/v) initially, proceeding to 35% acetonitrile/65% water (0.1% TFA, v/v) in a linear fashion after 9 min.) to give methyl 4-(8-(6-(2-methylpyrrolidin-1-yl)pyridin-2-ylamino)-[1,2,4]triazolo[1,5-a]pyridin-6-yl)benzoate 2,2,2-trifluoroacetate (0.015 g, 10.7%) as a yellow solid. ¹H NMR (300 MHz, CD₃OD): δ 9.02 (s, 1H), 8.59 (s, 1H), 8.18-8.15 (m, 2H), 7.88-7.77 (m, 4H), 6.53-6.44 (m, 2H), 4.39-4.35 (m, 1H), 3.95 (s, 3H), 3.85-3.79 (m, 1H), 3.64-3.59 (m, 1H), 2.32-2.21 (m, 3H), 1.96-1.93 (m, 1H), 1.33 (d, 3H, J=6.3 Hz). LC/MS: 429 [M+H]⁺. HPLC: 97.62% at 214 nm, 99.19% at 254 nm, t_{R}=6.545 min.

### Example 12

### 2-Methoxy-4-(3-(8-(6-(2-methylpyrrolidin-1-yl)pyridin-2-ylamino)-[1,2,4]triazolo[1,5-a]pyridin-6-yl)benzamido)benzoic acid hydrochloride

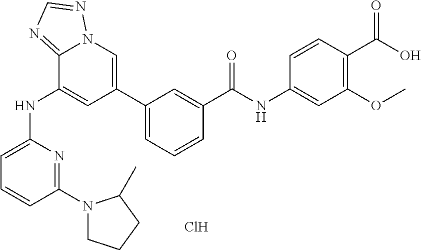

### Step 1

### 6-Chloro-N-(6-(2-methylpyrrolidin-1-yl)pyridin-2-yl)-[1,2,4]triazolo[1,5-a]pyridin-8-amine

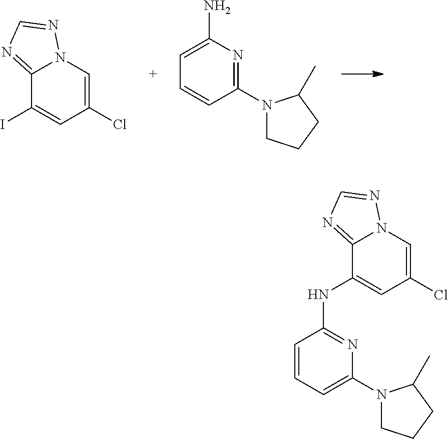

### Procedure:

To a stirred solution of 6-chloro-8-iodo-[1,2,4]triazolo[1,5-a]pyridine (0.8 g, 2.86 mmol), 6-(2-methylpyrrolidin-1-yl)pyridin-2-amine (0.60 g, 3.38 mmol), X-Phos (0.5 g, 1.05 mmol) and Cs₂CO₃ (3.2 g, 9.81 mmol) in xylene (50 mL) was added Pd₂(dba)₃ (0.245 g, 0.26 mmol) in one portion at room temperature under nitrogen. Then the mixture was degassed by bubbling nitrogen for 10 minutes and then heated at 140° C. under nitrogen for 16 h. The solvent was evaporated at 50° C. at reduced pressure and the residue was filtered through a plug of silica gel. The filter cake was washed with ethyl acetate/petroleum ether (1:4, v/v) then the combined filtrates were concentrated to give crude 6-chloro-N-(6-(2-methylpyrrolidin-1-yl)pyridin-2-yl)-[1,2,4]triazolo[1,5-a]pyridin-8-amine (0.55 g, 58%) as a yellow solid. This was used directly without further purification. LC/MS: 328.1 [M+H]⁺.

### Step 2

### Methyl 2-methoxy-4-(3-(4,4,5,5-tetramethyl-1,3,2-dioxaborolan-2-yl)benzamido)benzoate

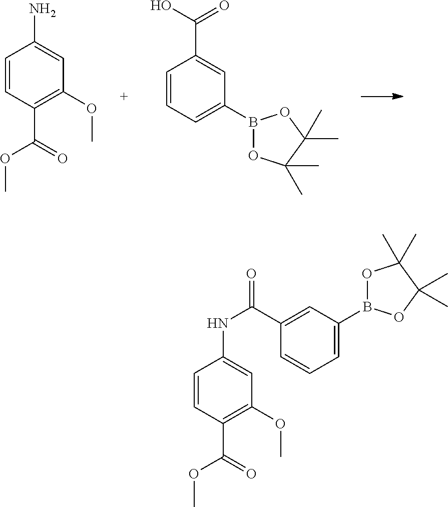

### Procedure:

To a stirred solution of 3-(4,4,5,5-tetramethyl-1,3,2-dioxaborolan-2-yl)benzoic acid (0.3 g, 1.2 mmol) in DMF (8 mL) were added DIEA (0.390 g, 3 mmol) and HATU (0.551 g, 1.45 mmol) successively at room temperature. Then methyl 4-amino-2-methoxybenzoate (0.219 g, 1.2 mmol) was added in one portion. The reaction mixture was stirred at room temperature for 72 h. The solvent was evaporated at 70° C. at reduced pressure and the residue was filtered through a plug of silica gel, washing the filter cake with petroleum ether/ethyl acetate (1:5, v/v). The combined filtrates were concentrated to give crude methyl 2-methoxy-4-(3-(4,4,5,5-tetramethyl-1,3,2-dioxaborolan-2-yl)benzamido)benzoate (0.220 g, 44%) as a white solid. This was used directly without further purification. LC/MS: 412.1 [M+H]⁺.

### Step 3

### Methyl 2-methoxy-4-(3-(8-(6-(2-methylpyrrolidin-1-yl)pyridin-2-ylamino)-[1,2,4]-triazolo[1,5-a]pyridin-6-yl)benzamido)benzoate

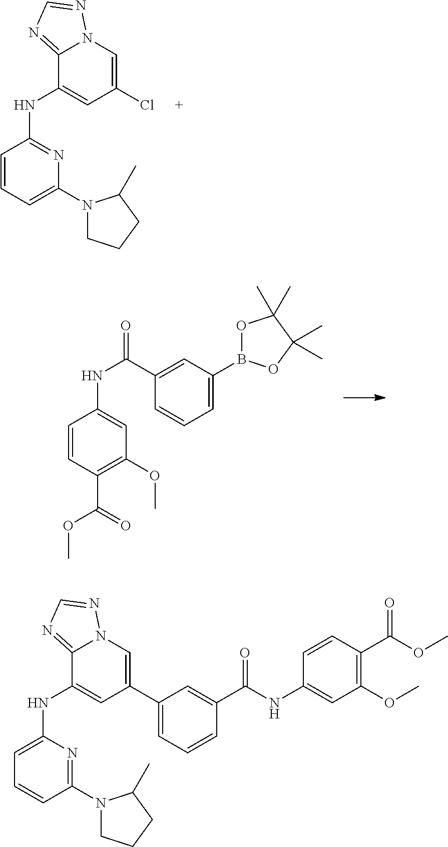

### Procedure:

To a stirred solution of 6-chloro-N-(6-(2-methylpyrrolidin-1-yl)pyridin-2-yl)-[1,2,4]triazolo[1,5-a]pyridin-8-amine (0.156 g, 0.47 mmol), methyl 2-methoxy-4-(3-(4,4,5,5-tetramethyl-1,3,2-dioxaborolan-2-yl)benzamido)benzoate (0.2 g, 0.48 mmol), X-Phos (0.086 g, 0.18 mmol), sodium carbonate (0.151 g, 1.42 mmol) and CsF (0.151 g, 1 mmol) in dioxane (20 mL) was added Pd₂(dba)₃ (0.043 g, 0.047 mmol) in one portion under nitrogen at room temperature. Then the reaction mixture was degassed by bubbling nitrogen and then heated at 98° C. for 16 h. The solvent was evaporated at 40° C. at reduced pressure and the residue was filtered through a plug of silica gel. The filter cake was washed with ethyl acetate/petroleum ether (1:1, v/v) and the combined filtrates concentrated to give crude methyl 2-methoxy-4-(3-(8-(6-(2-methylpyrrolidin-1-yl)pyridin-2-ylamino)-[1,2,4]triazolo[1,5-a]pyridin-6-yl)benzamido)benzoate (0.120 g, 43.7%) as a yellow solid. This was used directly without further purification. LC/MS: 578.1 [M+H]⁺

### Step 4

### 2-Methoxy-4-(3-(8-(6-(2-methylpyrrolidin-1-yl)pyridin-2-ylamino)-[1,2,4]triazolo[1,5-a]pyridin-6-yl)benzamido)benzoic acid hydrochloride

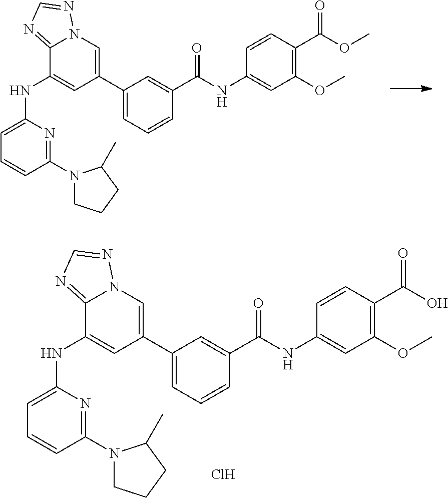

### Procedure:

To a stirred solution of methyl 2-methoxy-4-(3-(8-(6-(2-methylpyrrolidin-1-yl)pyridin-2-ylamino)-[1,2,4]triazolo[1,5-a]pyridin-6-yl)benzamido)benzoate (0.120 g, 0.20 mmol) in THF (20 mL) and methanol (5 mL) was added a solution of NaOH (0.083 g, 2 mmol) in water (2 mL) at room temperature. Then the reaction mixture was stirred at room temperature for 16 h. The solution was acidified by 0.1N HCl to pH=3. The solvent was evaporated at 40° C. at reduced pressure and the residue was purified by preparative-HPLC (Gemini 5u C18 150×21.2 mm; inject volume: 3 ml/inj, flow rate: 20 mL/min; wavelength: 214 nm and 254 nm; gradient conditions: 40% acetonitrile/60% water (0.1% TFA, v/v) initially, proceeding to 65% acetonitrile/35% water (0.1% TFA, v/v) in a linear fashion after 9 min). The desired product in solution was treated with conc. HCl to pH=1. The solvent was evaporated at 40° C. at reduced pressure to give 2-methoxy-4-(3-(8-(6-(2-methylpyrrolidin-1-yl)pyridin-2-ylamino)-[1,2,4]triazolo[1,5-a]pyridin-6-yl)benzamido)benzoic acid hydrochloride (0.025 g, 20%) as a yellow solid. ¹H NMR (300 MHz, CD₃OD): δ 9.05 (s, 1H), 8.61 (s, 1H), 8.32 (s, 1H), 8.06 (d, 1H, J=8.1 Hz), 7.99 (d, 1H, J=7.8 Hz), 7.91-7.68 (m, 6H), 7.68-7.38 (m, 1H), 6.54-6.45 (m, 1H), 4.41-4.38 (m, 1H), 3.96 (s, 3H), 3.95-3.80 (m, 1H), 3.71-3.55 (m, 1H), 2.28-2.24 (m, 3H), 2.03-1.93 (m, 1H), 1.34 (d, 3H, J=6.6 Hz). LC/MS: 564 [M+H]⁺, 562 [M−H]⁻. HPLC: 100% at 214 nm, 100% at 254 nm, t_{R}=6.31 min.

### Example 13

### Methyl 3-(8-(6-(2-methylpyrrolidin-1-yl)pyridin-2-ylamino)-[1,2,4]triazolo[1,5-a]pyridin-6-yl)benzoate

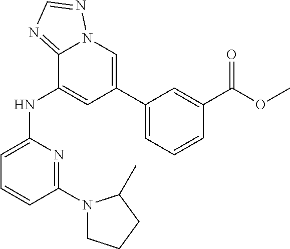

### Step 1

### 3-(8-(6-(2-Methylpyrrolidin-1-yl)pyridin-2-ylamino)-[1,2,4]triazolo[1,5-a]pyridin-6-yl)benzoate

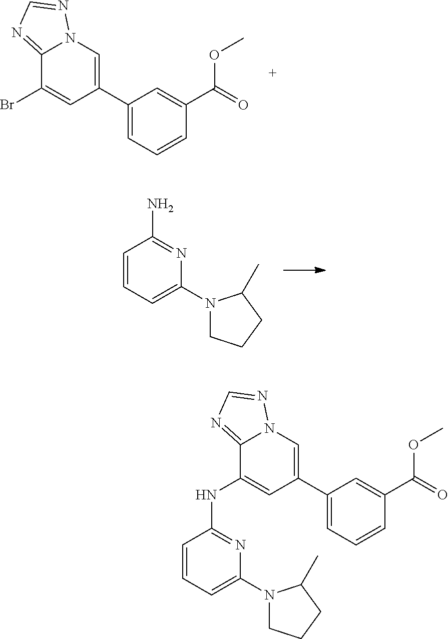

### Procedure:

To a stirred solution of methyl 3-(8-bromo-[1,2,4]triazolo[1,5-a]pyridin-6-yl)benzoate (0.18 g, 0.54 mmol), 6-(2-methylpyrrolidin-1-yl)pyridin-2-amine (0.144 g, 0.81 mmol), BINAP (0.13 g, 0.20 mmol) and Cs₂CO₃ (0.50 g, 1.53 mmol) in dioxane (18 mL) was added Pd₂(dba)₃ (0.05 g, 0.054 mmol) in one portion at room temperature under nitrogen. Then the mixture was degassed by bubbling nitrogen for 10 minutes and then stirred at 96° C. under nitrogen for 16 h. The solvent was evaporated at 40° C. at reduced pressure and the residue was purified by column chromatography (silica gel, 200-300 mesh, ethyl acetate/petroleum ether 1:1, v/v) to give methyl 3-(8-(6-(2-methylpyrrolidin-1-yl)pyridin-2-ylamino)-[1,2,4]triazolo[1,5-a]pyridin-6-yl)benzoate as an impure product. This was further purified by preparative-HPLC (Gemini 5u C18 150×21.2 mm; inject volume: 3 ml/inj, flow rate: 20 mL/min; wavelength: 214 nm and 254 nm; gradient conditions: 53% acetonitrile/47% water (0.1% TFA, v/v) initially, proceed to 61% acetonitrile/39% water (0.1% TFA, v/v) in a linear fashion after 9 min.) to give methyl 3-(8-(6-(2-methylpyrrolidin-1-yl)pyridin-2-ylamino)-[1,2,4]triazolo[1,5-a]pyridin-6-yl)benzoate 222-trifluoroacetate (0.032 g, 13.8%) as a yellow solid. ¹H NMR (300 MHz, CDCl₃): δ 8.82 (s, 1H), 8.43 (s, 1H), 8.31 (s, 1H), 8.16 (d, 1H, J=7.8 Hz), 8.07 (s, 1H), 7.84 (d, 1H, J=7.5 Hz), 7.63 (t, 1H, J=7.8 Hz), 7.60-7.50 (m, 2H), 6.08 (d, 1H, J=8.7 Hz), 6.00 (d, 1H, J=7.8 Hz), 4.32 (brs, 1H), 3.80 (brs, 1H), 3.58-3.54 (m, 1H), 2.21 (brs, 3H), 1.87 (brs, 1H), 1.25 (d, 3H, J=7.5 Hz). LC/MS: 429 [M+H]⁺. HPLC: 100% at 214 nm, 100% at 254 nm, t_{R}=7.26 min.

### Example 14

### 4-(8-(6-(2-Methylpyrrolidin-1-yl)pyridin-2-ylamino)-[1,2,4]triazolo[1,5-a]pyridin-6-yl)benzoic acid

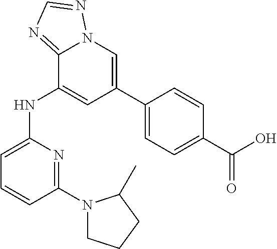

### Step 1

### Methyl 4-(8-(6-(2-methylpyrrolidin-1-yl)pyridin-2-ylamino)[1,2,4]triazolo[1,5-a]pyridin-6-yl)benzoate

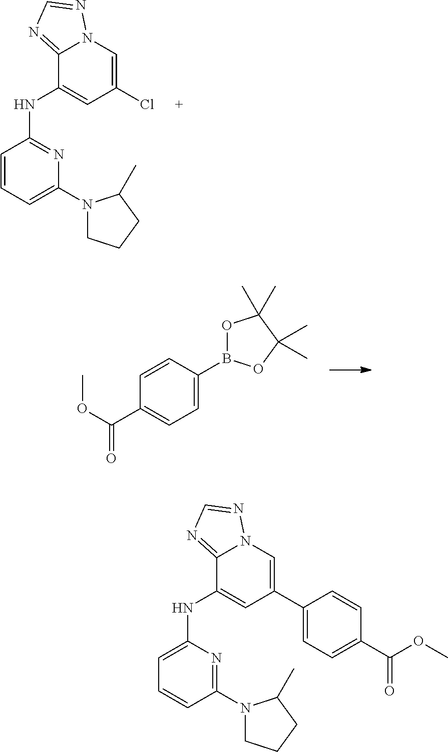

### Procedure:

To a stirred solution of 6-chloro-N-(6-(2-methylpyrrolidin-1-yl)pyridin-2-yl)-[1,2,4]triazolo[1,5-a]pyridin-8-amine (0.126 g, 0.38 mmol), methyl 4-(4,4,5,5-tetramethyl-1,3,2-dioxaborolan-2-yl)benzoate (0.151 g, 0.57 mmol), X-Phos (0.052 g, 0.11 mmol), sodium carbonate (0.120 g, 1.13 mmol) in water (1 mL) and dioxane (15 mL) was added Pd₂(dba)₃ (0.026 g, 0.028 mmol) in one portion under nitrogen at room temperature. Then the reaction mixture was degassed by bubbling nitrogen for 10 minutes and then stirred at 97° C. for 16 h under nitrogen. The solvent was evaporated at 40° C. at reduced pressure and the residue filtered through a plug of silica gel. The cake was washed with ethyl acetate/petroleum ether (1:3, v/v) then the combined filtrates concentrated to give crude methyl 4-(8-(6-(2-methylpyrrolidin-1-yl)pyridin-2-ylamino)-[1,2,4]triazolo[1,5-a]pyridin-6-yl)benzoate (0.150 g, 91.4%) as a yellow solid. This was used directly without further purification. LC/MS: 429.1 [M+H]⁺.

### Step 2

### 4-(8-(6-(2-Methylpyrrolidin-1-yl)pyridin-2-ylamino)-[1,2,4]triazolo[1,5-a]pyridin-6-yl)benzoic acid

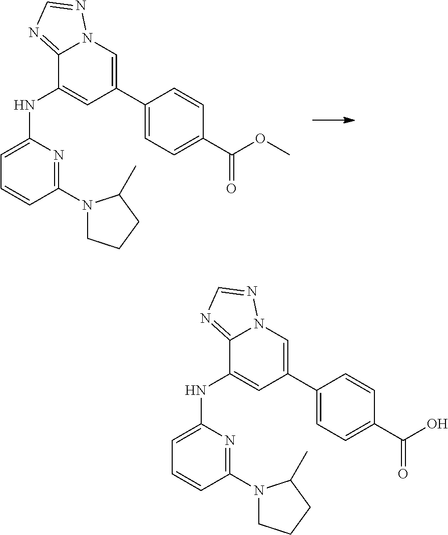

### Procedure:

To a stirred solution of methyl 4-(8-(6-(2-methylpyrrolidin-1-yl)pyridin-2-ylamino)-[1,2,4]triazolo[1,5-a]pyridin-6-yl)benzoate (0.150 g, 0.35 mmol) in methanol (2 mL) and THF (10 mL) was added a solution of NaOH (0.070 g, 1.75 mmol) in water (2 mL) at room temperature. Then the reaction mixture was stirred at room temperature for 16 h. The solution was acidified by 1N HCl to pH=3. The solvent was evaporated at 40° C. at reduced pressure and the residue was purified by column chromatography (silica gel, 200-300 mesh, ethyl acetate/methanol 10:1, v/v) to give 4-(8-(6-(2-methylpyrrolidin-1-yl)pyridin-2-ylamino)-[1,2,4]triazolo[1,5-a]pyridin-6-yl)benzoic acid (0.125 g, 86.2%) as a yellow solid. ¹H NMR (300 MHz, CDCl₃): δ 8.82 (s, 1H), 8.43 (s, 1H), 8.31 (s, 1H), 8.16 (d, 1H, J=7.8 Hz), 8.07 (s, 1H), 7.84 (d, 1H, J=7.5 Hz), 7.63 (t, 1H, J=7.8 Hz), 7.60-7.50 (m, 2H), 6.08 (d, 1H, J=8.7 Hz), 6.00 (d, 1H, J=7.8 Hz), 4.32 (brs, 1H), 3.80 (brs, 1H), 3.58-3.54 (m, 1H), 2.21 (brs, 3H), 1.87 (brs, 1H), 1.25 (d, 3H, J=7.5 Hz) LC/MS: 429 [M+H]⁺. HPLC: 100% at 214 nm, 100% at 254 nm, t_{R}=7.26 min.

### Example 15

### 3-(8-(6-(2-Methylpyrrolidin-1-yl)pyridin-2-ylamino)-[1,2,4]triazolo[1,5-a]pyridin-6-yl)benzoic acid

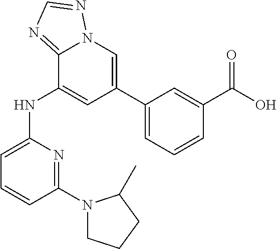

### Step 1

### 4-(8-(6-(2-Methylpyrrolidin-1-yl)pyridin-2-ylamino)-[1,2,4]triazolo[1,5-a]pyridin-6-yl)benzoic acid

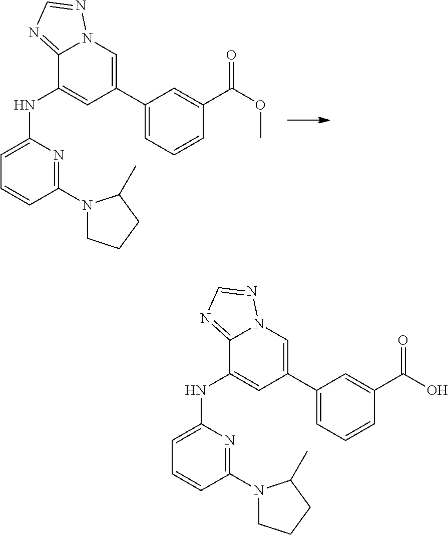

### Procedure:

To a stirred solution of methyl 3-(8-(6-(2-methylpyrrolidin-1-yl)pyridin-2-ylamino)-[1,2,4]triazolo[1,5-a]pyridin-6-yl)benzoate (0.14 g, 0.327 mmol) in methanol (2 mL) and THF (10 mL) was added a solution of NaOH (0.065 g, 1.635 mmol) in water (2 mL) at room temperature. Then the reaction mixture was stirred at room temperature for 16 h. The solution was acidified by 1N HCl to pH=2. The solvent was evaporated at 40° C. at reduced pressure and the residue was purified by column chromatography (silica gel, 200-300 mesh, ethyl acetate/methanol 10:1, v/v) to give 3-(8-(6-(2-methylpyrrolidin-1-yl)pyridin-2-ylamino)-[1,2,4]triazolo[1,5-a]pyridin-6-yl)benzoic acid (0.078 g, 57.6%) as a yellow solid. ¹H NMR (300 MHz, DMSO): δ 9.14 (s, 1H), 9.07 (s, 1H), 8.79 (s, 1H), 8.53 (s, 1H), 8.27 (s, 1H), 8.03-8.00 (m, 1H), 7.64 (t, 1H, J=7.8 Hz), 7.40 (t, 1H, J=7.9 Hz), 6.62 (d, 1H, J=7.8 Hz), 5.99 (d, 1H, J=8.1 Hz), 4.17 (brs, 1H), 3.52 (brs, 1H), 3.40-3.35 (m, 1H), 2.03-1.92 (m, 3H), 1.62 (brs, 1H), 1.04 (d, 3H, J=6.0 Hz). LC/MS: 415 [M+H]⁺, 413 [M−H]⁻. HPLC: 99.35% at 214 nm, 99.22% at 254 nm, t_{R}=5.82 min.

### Example 16

### 4-(8-(5,6-Dimethoxypyridin-2-ylamino)-[1,2,4]triazolo[1,5-a]pyridin-6-yl)-N-(2-(dimethylamino)ethyl)benzamide hydrochloride

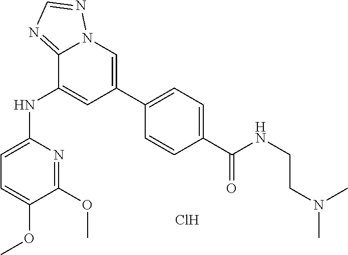

### Step 1

### N-(2-(Dimethylamino)ethyl)-4-(4,4,5,5-tetramethyl-1,3,2-dioxaborolan-2-yl)benzamide

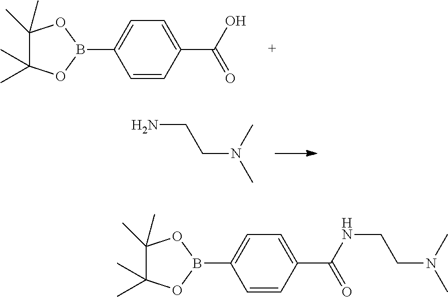

### Procedure

A mixture of 4-(4,4,5,5-tetramethyl-1,3,2-dioxaborolan-2-yl)benzoic acid (300 mg, 1.2 mmol), N,N-dimethylethane-1,2-diamine (128 mg, 1.45 mmol), EDCI (690 mg, 3.6 mmol) and 1-methyl-1H-imidazole (393 mg, 4.8 mmol) in dichloromethane (50 mL) were stirred at room temperature overnight. The solvent was washed with saturated NaHCO₃ (aq., 30 mL), H₂O (30 mL) and brine (30 mL), then dried over Na₂SO₄. The organic phase was concentrated under reduced pressure to give N-(2-(dimethylamino)ethyl)-4-(4,4,5,5-tetramethyl-1,3,2-dioxaborolan-2-yl)benzamide (250 mg) as crude product which was used directly in the next step without further purification. LC/MS: 319 [M+H]⁺.

### Step 2

### 4-(8-(5,6-Dimethoxypyridin-2-ylamino)-[1,2,4]-triazolo[1,5-a]pyridin-6-yl)-N-(2-(dimethylamino)ethyl)benzamide hydrochloride

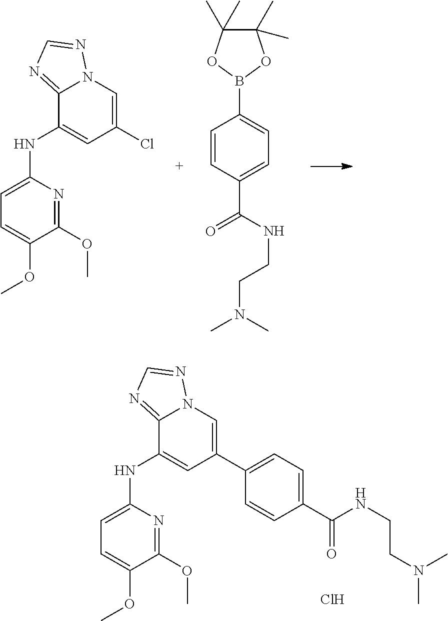

### Procedure:

A mixture of 6-chloro-N-(5,6-dimethoxypyridin-2-yl)-[1,2,4]triazolo[1,5-a]pyridin-8-amine (100 mg, 0.33 mmol), N-(2-(dimethylamino)ethyl)-4-(4,4,5,5-tetramethyl-1,3,2-dioxaborolan-2-yl)benzamide (126 mg, 0.4 mmol), Pd₂(dba)₃ (30 mg, 0.05 mmol), X-Phos (30 mg, 0.06 mmol) and Na₂CO₃ (70 mg, 0.66 mmol) in the mixture of dioxane and H₂O (30 mL/5 mL) were stirred at reflux for 18 h under N₂ atmosphere. The solvent was removed under reduce pressure and the residue was washed with 15 mL of H₂O, and then purified by preparative-HPLC (Gemini 5u C18 150×21.2 mm; inject volume: 3 ml/inj, flow rate: 20 mL/min; wavelength: 214 nm and 254 nm; gradient conditions: 20% acetonitrile/80% water (0.1% TFA, v/v) initially, proceeding to 45% acetonitrile/55% water (0.1% TFA, v/v) in a linear fashion after 9 min.). The solution of the purified compound was adjusted to pH=2 with 1N HCl and then concentrated under reduced pressure to give 4-(8-(5,6-dimethoxypyridin-2-ylamino)-[1,2,4]triazolo[1,5-a]pyridin-6-yl)-N-(2-(dimethylamino)ethyl)benzamide hydrochloride (32 mg, 21%) as a yellow solid. ¹H NMR (300 MHz, DMSO): δ 10.04 (brs, 1H), 9.38 (s, 1H), 8.95-8.86 (m, 3H), 8.57 (s, 1H), 8.07-8.05 (m, 2H), 7.93-7.90 (m, 2H), 7.41 (d, 1H, J=8.4 Hz), 7.02 (d, 1H, J=8.4 Hz), 3.98 (s, 3H), 3.77 (s, 3H), 3.69-3.67 (m, 2H), 3.32 (m, 2H), 2.87 (s, 3H), 2.86 (s, 3H). LC/MS: 462 [M+H]⁺. HPLC: 98.12% at 214 nm, 99.83% at 254 nm, t_{R}=5.20 min.

### Example 17

### 4-(8-(5,6-Dimethoxypyridin-2-ylamino)-[1,2,4]-triazolo[1,5-a]pyridin-6-yl)benzamide

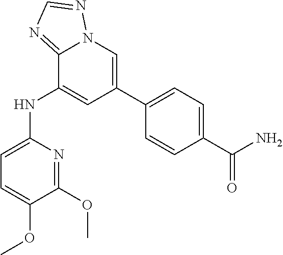

### Step 1

### 4-(8-(5,6-Dimethoxypyridin-2-ylamino)-[1,2,4]-triazolo[1,5-a]pyridin-6-yl)benzamide

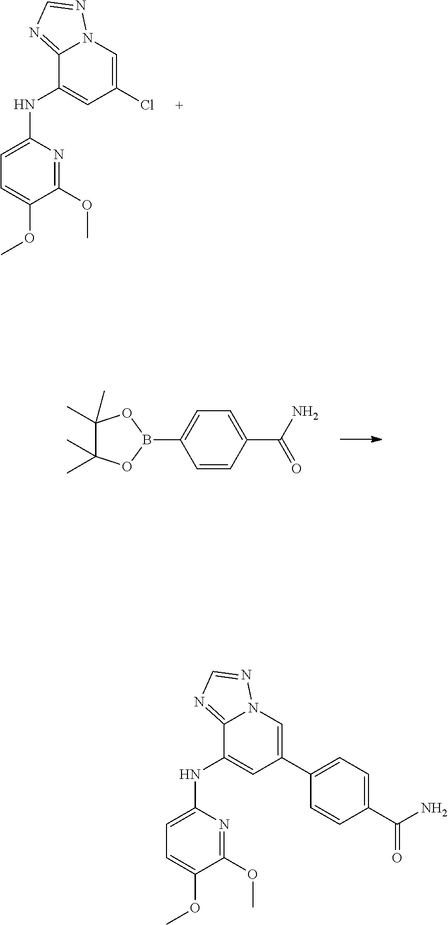

### Procedure:

A mixture of 6-chloro-N-(5,6-dimethoxypyridin-2-yl)-[1,2,4]triazolo[1,5-a]pyridin-8-amine (100 mg, 0.33 mmol), 4-(4,4,5,5-tetramethyl-1,3,2-dioxaborolan-2-yl)benzamide (100 mg, 0.4 mmol), Pd₂(dba)₃ (30 mg, 0.05 mmol), X-Phos (30 mg, 0.06 mmol) and Cs₂CO₃ (215 mg, 0.66 mmol) in dioxane/H₂O (30 mL/5 mL) were stirred at reflux for 18 h under N₂ atmosphere. The solvent was removed under reduced pressure and then the residue purified by preparative-HPLC (Gemini 5u C18 150×21.2 mm; inject volume: 3 ml/inj, flow rate: 20 mL/min; wavelength: 214 nm and 254 nm; gradient conditions: 30% acetonitrile/70% water (0.1% TFA, v/v) initially, proceed to 55% acetonitrile/45% water (0.1% TFA, v/v) in a linear fashion after 9 min.) to give 4-(8-(5,6-dimethoxypyridin-2-ylamino)-[1,2,4]triazolo[1,5-a]pyridin-6-yl)benzamide 222-trifluoroacetate (25 mg, 20%) as a yellow solid. ¹H NMR (300 MHz, DMSO): δ 9.35 (s, 1H), 8.93 (s, 1H), 8.84 (s, 1H), 8.55 (s, 1H), 8.03-8.00 (m, 3H), 7.88-7.86 (m, 2H), 7.42-7.39 (m, 2H), 7.02 (d, 1H, J=8.4 Hz), 3.98 (s, 3H), 3.77 (s, 3H). LC/MS: 391 [M+H]⁺. HPLC: 98.88% at 214 nm, 99.38% at 254 nm, t_{R}=5.13 min.

### Example 18

### (S)-4-(8-(6-(2-Methylpyrrolidin-1-yl)pyridin-2-ylamino)-[1,2,4]-triazolo[1,5-a]pyridin-6-yl)benzamide

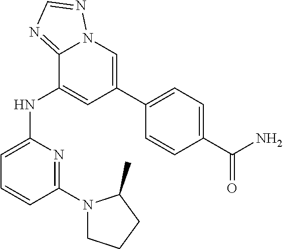

### Step 1

### (S)-6-Chloro-N-(6-(2-methylpyrrolidin-1-yl)pyridin-2-yl)-[1,2,4]-triazolo[1,5-a]pyridin-8-amine

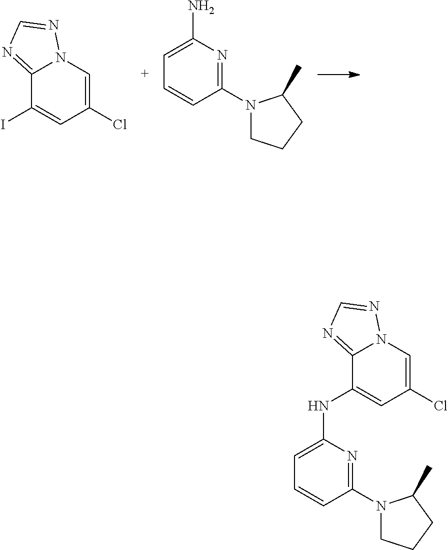

### Procedure:

A mixture of 6-chloro-8-iodo-[1,2,4]triazolo[1,5-a]pyridine (700 mg, 2.5 mmol), (S)-6-(2-methylpyrrolidin-1-yl)pyridin-2-amine (490 mg, 2.75 mmol), Pd₂(dba)₃ (100 mg, 0.17 mmol), X-Phos (100 mg, 0.21 mmol) and Cs₂CO₃ (1.6 g, 5.0 mmol) in xylene (30 mL) was stirred at 140° C. for 8 h under N₂ atmosphere. The solvent was removed under reduced pressure and then the residue was purified by column chromatography (silica gel, 200-300 mesh, EtOAc/petroleum ether 1/1, v/v) to give (S)-6-chloro-N-(6-(2-methylpyrrolidin-1-yl)pyridin-2-yl)-[1,2,4]triazolo[1,5-a]pyridin-8-amine (450 mg, 55%) as a yellow solid. ¹H NMR (300 MHz, DMSO): δ 9.34 (s, 1H), 8.94 (d, 1H, J=1.5 Hz), 8.71 (d, 1H, J=2.1 Hz), 8.51 (s, 1H), 7.37 (t, 1H, J=8.1 Hz), 6.61 (d, 1H, J=8.1 Hz), 5.97 (d, 1H, J=8.1 Hz), 4.23-4.18 (m, 1H), 3.51-3.46 (m, 1H), 3.34-3.25 (m, 1H), 2.13-1.94 (m, 3H), 1.75-1.69 (m, 1H), 1.25 (d, 1H, J=6.3 Hz). LC/MS: 329.1 [M+H]⁺.

### Step 2

### (S)-4-(8-(6-(2-Methylpyrrolidin-1-yl)pyridin-2-ylamino)-[1,2,4]triazolo[1,5-a]pyridin-6-yl)benzamide

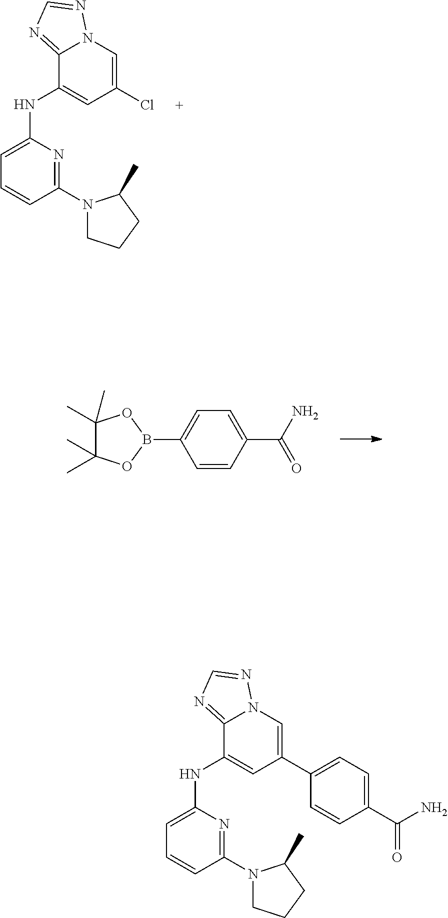

### Procedure:

A mixture of (S)-6-chloro-N-(6-(2-methylpyrrolidin-1-yl)pyridin-2-yl)-[1,2,4]triazolo[1,5-a]pyridin-8-amine (100 mg, 0.3 mmol), 4-(4,4,5,5-tetramethyl-1,3,2-dioxaborolan-2-yl)benzamide (89 mg, 0.36 mmol), Pd(dba)₂ (30 mg, 0.05 mmol), X-Phos (30 mg, 0.06 mmol) and Cs₂CO₃ (196 mg, 0.6 mmol) in dioxane/H₂O (30 mL/5 mL) were stirred at reflux for 18 h under N₂ atmosphere. The solvent was removed under reduced pressure and then the residue purified by preparative-HPLC (Gemini 5u C18 150×21.2 mm; inject volume: 3 ml/inj, flow rate: 20 mL/min; wavelength: 214 nm and 254 nm; gradient conditions: 23% acetonitrile/77% water (0.1% TFA, v/v) initially, proceed to 55% acetonitrile/45% water (0.1% TFA, v/v) in a linear fashion after 9 min.) to give (S)-4-(8-(6-(2-methylpyrrolidin-1-yl)pyridin-2-ylamino)-[1,2,4]triazolo[1,5-a]pyridin-6-yl)benzamide 222-trifluoroacetate (25 mg, 20%) as a yellow solid. ¹H NMR (300 MHz, DMSO): δ 9.50 (brs, 2H, exchangeable by D₂O), 8.94 (s, 1H), 8.91 (s, 1H), 8.61 (s, 1H), 8.12 (brs, 1H, exchangeable by D₂O), 8.05-8.02 (m, 2H), 7.89-7.86 (m, 2H), 7.53-7.48 (m, 2H, 1H exchangeable by D₂O), 6.64 (d, 1H, J=7.8 Hz, exchangeable by D₂O), 6.11 (d, 1H, J=8.4 Hz, exchangeable by D₂O), 4.24 (brs, 1H), 3.59 (brs, 1H), 3.42-3.40 (m, 1H), 2.20-2.01 (m, 2H), 1.78-1.72 (m, 1H), 1.13 (d, 1H, J=6.0 Hz). LC/MS: 411 [M+H]⁺. HPLC: 100% at 214 nm, 100% at 254 nm, t_{R}=5.15 min.

### Example 19

### (3-(8-(5,6-Dimethoxypyridin-2-ylamino)-[1,2,4]-triazolo[1,5-a]pyridin-6-yl)phenyl)methanol

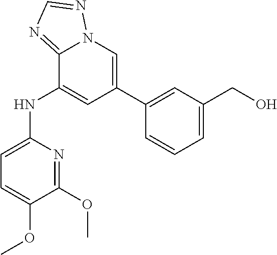

### Step 1

### Methyl 3-(8-(5,6-dimethoxypyridin-2-ylamino)-[1,2,4]-triazolo[1,5-a]pyridin-6-yl)benzoate

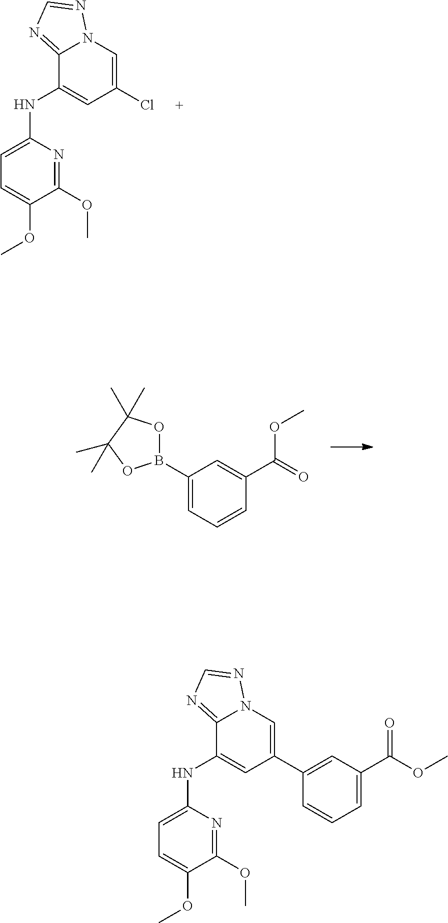

### Procedure:

A mixture of 6-chloro-N-(5,6-dimethoxypyridin-2-yl)-[1,2,4]triazolo[1,5-a]pyridin-8-amine (100 mg, 0.33 mmol), methyl 3-(4,4,5,5-tetramethyl-1,3,2-dioxaborolan-2-yl)benzoate (105 mg, 0.4 mmol), Pd₂(dba)₃ (30 mg, 0.05 mmol), X-Phos (30 mg, 0.06 mmol) and Na₂CO₃ (70 mg, 0.66 mmol) in dioxane/H₂O (30 mL/5 mL) were stirred at reflux for 18 h under N₂ atmosphere. The solvent was removed under reduce pressure and then the residue filtered through a plug of silica gel. The cake was washed with EtOAc and the combined filtrates were concentrated to give crude methyl 3-(8-(5,6-dimethoxypyridin-2-ylamino)-[1,2,4]triazolo[1,5-a]pyridin-6-yl)benzoate (60 mg, 45%) as a white solid. This was used directly without further purification. LC/MS: 406 [M+H]⁺.

### Step 2

### (3-(8-(5,6-Dimethoxypyridin-2-ylamino)-[1,2,4]-triazolo[1,5-a]pyridin-6-yl)phenyl)methanol

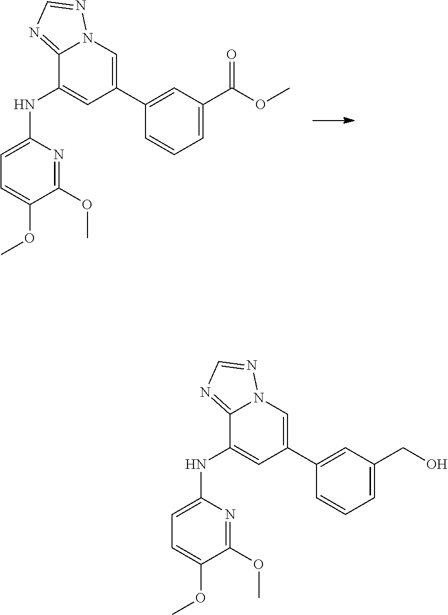

### Procedure:

To a suspension of LiAlH₄ (50 mg, 1.3 mmol) in THF (30 mL), was added in one portion at room temperature methyl 3-(8-(5,6-dimethoxypyridin-2-ylamino)-[1,2,4]triazolo[1,5-a]pyridin-6-yl)benzoate (60 mg, 0.15 mmol) then the mixture was stirred for 4 h. The reaction was quenched by adding H₂O (3 mL) with cooling in an ice-water bath, then the mixture was filtered and concentrated. The crude residue was purified by preparative-HPLC (Gemini 5u C18 150×21.2 mm; inject volume: 3 ml/inj, flow rate: 20 mL/min; wavelength: 214 nm and 254 nm; gradient conditions: 40% acetonitrile/60% water (0.1% TFA, v/v) initially, proceed to 70% acetonitrile/30% water (0.1% TFA, v/v) in a linear fashion after 9 min.) to give (3-(8-(5,6-dimethoxypyridin-2-ylamino)-[1,2,4]triazolo[1,5-a]pyridin-6-yl)phenyl)methanol 2,2,2-trifluoroacetate (25 mg, 43%) as a yellow solid. ¹H NMR (300 MHz, DMSO): δ 9.32 (s, 1H), 8.92 (s, 1H), 8.71 (s, 1H), 8.53 (s, 1H), 7.73 (brs, 1H), 7.66 (d, 1H, J=7.5 Hz), 7.49-7.36 (m, 3H), 7.02 (d, 1H, J=8.4 Hz), 4.60 (s, 2H), 4.00 (s, 3H), 3.77 (s, 3H). LC/MS: 378 [M+H]⁺, 376 [M−H]⁻. HPLC: 98.45% at 214 nm, 99.59% at 254 nm, t_{R}=5.81 min.

### Example 20

### (S)—N-(2-(Dimethylamino)ethyl)-4-(8-(6-(2-methylpyrrolidin-1-yl)pyridin-2-ylamino)-[1,2,4]triazolo[1,5-a]pyridin-6-yl)benzamide 2,2,2-trifluoroacetate

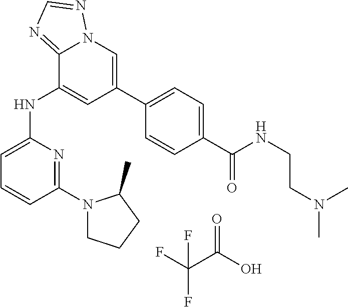

### Step 1

### (S)—N-(2-(Dimethylamino)ethyl)-4-(8-(6-(2-methylpyrrolidin-1-yl)pyridin-2-ylamino)-[1,2,4]triazolo[1,5-a]pyridin-6-yl)benzamide-2,2,2-trifluoroacetate

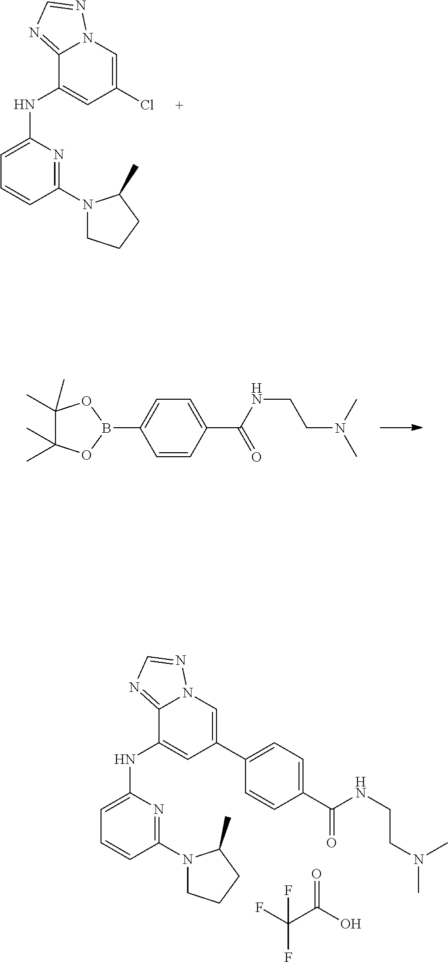

### Procedure:

A mixture of (S)-6-chloro-N-(6-(2-methylpyrrolidin-1-yl)pyridin-2-yl)-[1,2,4]triazolo[1,5-a]pyridin-8-amine (100 mg, 0.3 mmol), N-(2-(dimethylamino)ethyl)-4-(4,4,5,5-tetramethyl-1,3,2-dioxaborolan-2-yl)benzamide (114 mg, 0.36 mmol), Pd₂(dba)₃ (30 mg, 0.05 mmol), X-Phos (30 mg, 0.06 mmol) and Cs₂CO₃ (196 mg, 0.6 mmol) in dioxane/H₂O (30 mL/5 mL) were stirred at reflux for 18 h under N₂ atmosphere. The solvent was removed under reduced pressure and the residue purified by preparative-HPLC (Gemini 5u C18 150×21.2 mm; inject volume: 3 ml/inj, flow rate: 20 mL/min; wavelength: 214 nm and 254 nm; gradient conditions: 20% acetonitrile/80% water (0.1% TFA, v/v) initially, proceed to 60% acetonitrile/40% water (0.1% TFA, v/v) in a linear fashion after 9 min.) to give (S)—N-(2-(dimethylamino)ethyl)-4-(8-(6-(2-methylpyrrolidin-1-yl)pyridin-2-ylamino)-[1,2,4]triazolo[1,5-a]pyridin-6-yl)benzamide 2,2,2-trifluoroacetate (26 mg, 14%) as a yellow solid. ¹H NMR (300 MHz, DMSO): δ 9.25 (brs, 1H, exchangeable by D₂O), 9.16 (s, 1H), 9.15 (s, 1H), 8.59 (s, 1H), 8.81-8.79 (m, 1H, exchangeable by D₂O), 8.57 (s, 1H), 8.04-8.01 (m, 2H), 7.93-7.91 (m, 2H), 7.42 (t, 1H, J=8.4 Hz), 6.64 (d, 1H, J=7.8 Hz, exchangeable by D₂O), 6.00 (d, 1H, J=8.4 Hz, exchangeable by D₂O), 4.22 (brs, 1H), 3.67-3.55 (m, 3H), 3.40-3.31 (m, 3H), 2.91 (s, 3H), 2.89 (s, 3H), 2.07-1.96 (m, 3H), 1.67 (brs, 1H), 1.10 (d, 3H, J=6.0 Hz). LC/MS: 486 [M+H]⁺, 244 [M/2+H]⁺. HPLC: 99.24% at 214 nm, 99.56% at 254 nm, t_{R}=4.76 min.

### Example 21

### (S)-(3-(8-(6-(2-Methylpyrrolidin-1-yl)pyridin-2-ylamino)-1,2,41-triazolo[1,5-a]pyridin-6-yl)phenyl)methanol

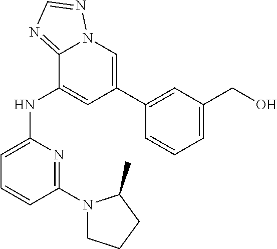

### Step 1

### (3-(4,4,5,5-Tetramethyl-1,3,2-dioxaborolan-2-yl)phenyl)methanol

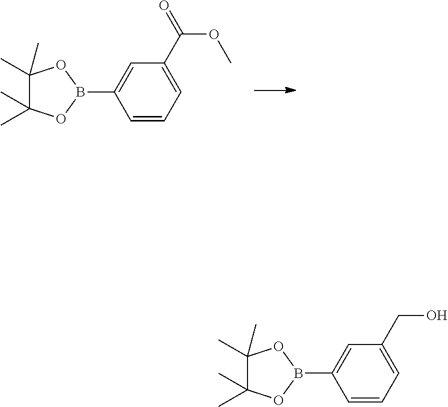

### Procedure:

To a suspension of LiAlH₄ (100 mg, 2.63 mmol) in THF (30 mL) was added portion-wise at room temperature methyl 3-(4,4,5,5-tetramethyl-1,3,2-dioxaborolan-2-yl)benzoate (200 mg, 0.76 mmol). The reaction mixture was stirred at room temperature for additional 3 h. H₂O (3 mL) was added dropwise at 0° C. then the mixture was filtered and concentrated under reduced pressure to give crude (3-(4,4,5,5-tetramethyl-1,3,2-dioxaborolan-2-yl)phenyl)methanol (190 mg) which was used for next step without characterization or purification.

### Step 2

### (S)-(3-(8-(6-(2-Methylpyrrolidin-1-yl)pyridin-2-ylamino)-[1,2,4]triazolo[1,5-a]pyridin-6-yl)phenyl)methanol

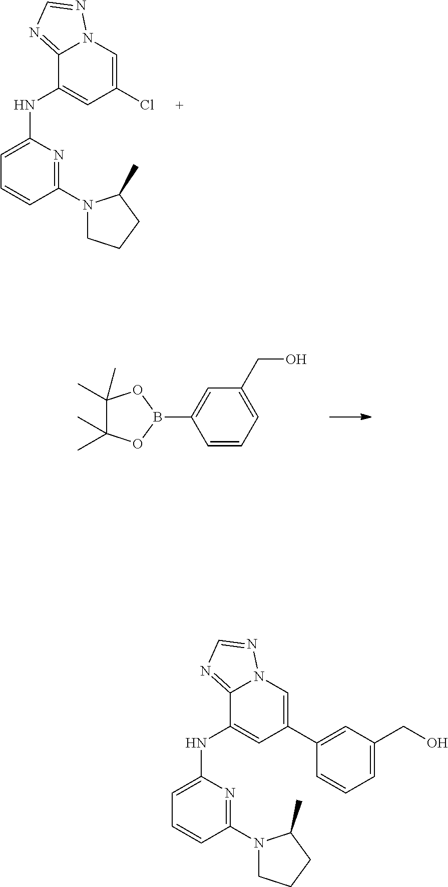

### Procedure:

A mixture of (S)-6-chloro-N-(6-(2-methylpyrrolidin-1-yl)pyridin-2-yl)-[1,2,4]triazolo[1,5-a]pyridin-8-amine (100 mg, 0.3 mmol), (3-(4,4,5,5-tetramethyl-1,3,2-dioxaborolan-2-yl)phenyl)methanol (84 mg, 0.36 mmol), Pd₂(dba)₃ (30 mg, 0.05 mmol), X-Phos (30 mg, 0.06 mmol) and Cs₂CO₃ (196 mg, 0.6 mmol) in dioxane/H₂O (30 mL/5 mL) was stirred at reflux for 18 h under N₂ atmosphere. The solvent was removed under reduced pressure and the residue purified by preparative-HPLC (Gemini 5u C18 150×21.2 mm; inject volume: 3 ml/inj, flow rate: 20 mL/min; wavelength: 214 nm and 254 nm; gradient conditions: 50% acetonitrile/50% water (0.1% TFA, v/v) initially, and proceed to 82% acetonitrile/18% water (0.1% TFA, v/v) in a linear fashion after 9 min.) to give (S)-(3-(8-(6-(2-methylpyrrolidin-1-yl)pyridin-2-ylamino)-[1,2,4]triazolo[1,5-a]pyridin-6-yl)phenyl)methanol 222-trifluoroacetate (25 mg, 21%) as a yellow solid. ¹H NMR (300 MHz, CD₃OD): δ 9.18 (s, 1H), 8.42 (s, 1H), 8.34 (s, 1H), 7.67 (s, 1H), 7.60-7.56 (m, 1H), 7.49-7.35 (m, 3H), 6.23 (d, 1H, J=7.8 Hz), 5.98 (d, 1H, J=8.1 Hz), 4.70 (s, 2H), 4.24-4.20 (m, 1H), 3.59-3.55 (m, 1H), 3.41-3.38 (m, 1H), 2.13-1.98 (m, 3H), 1.72-1.70 (m, 1H), 1.13 (d, 3H, J=6.3 Hz). LC/MS: 401 [M+H]⁺. HPLC: 100% at 214 nm, 100% at 254 nm, t_{R}=5.54 min.

### Example 22

### (S)-2-(3-(8-(6-(2-Methylpyrrolidin-1-yl)pyridin-2-ylamino)-[1,2,4]triazolo[1,5-a]pyridin-6-yl)benzylamino)ethanol hydrochloride

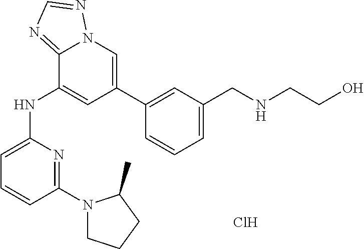

### Step 1

### 2-(3-(4,4,5,5-Tetramethyl-1,3,2-dioxaborolan-2-yl)benzylamino)ethanol

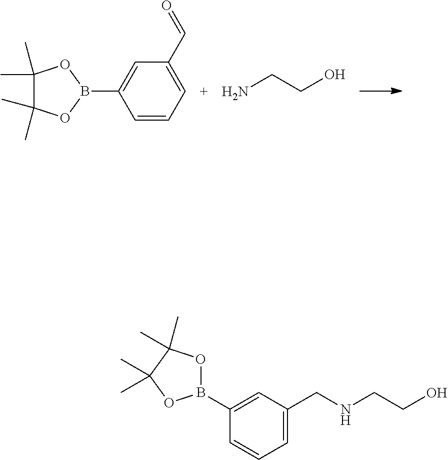

### Procedure:

A mixture of 3-(4,4,5,5-tetramethyl-1,3,2-dioxaborolan-2-yl)benzaldehyde (200 mg, 0.86 mmol), 2-aminoethanol (63 mg, 1.0 mmol), sodium triacetoxyborohydride (547 mg, 2.58 mmol) and HOAc (0.1 mL) in DCM (50 mL) was stirred at room temperature overnight. The mixture was washed with H₂O (3×30 mL) and the organic layer was dried over Na₂SO₄, then concentrated under reduced pressure to give crude 2-(3-(4,4,5,5-tetramethyl-1,3,2-dioxaborolan-2-yl)benzylamino)ethanol (200 mg) which was used directly in the next step without further purification. LC/MS: 278 [M+H]⁺, t_{R}=1.20 min.

### Step 2

### (S)-2-(3-(8-(6-(2-Methylpyrrolidin-1-yl)pyridin-2-ylamino)-[1,2,4]triazolo[1,5-a]pyridin-6-yl)benzylamino)ethanol hydrochloride

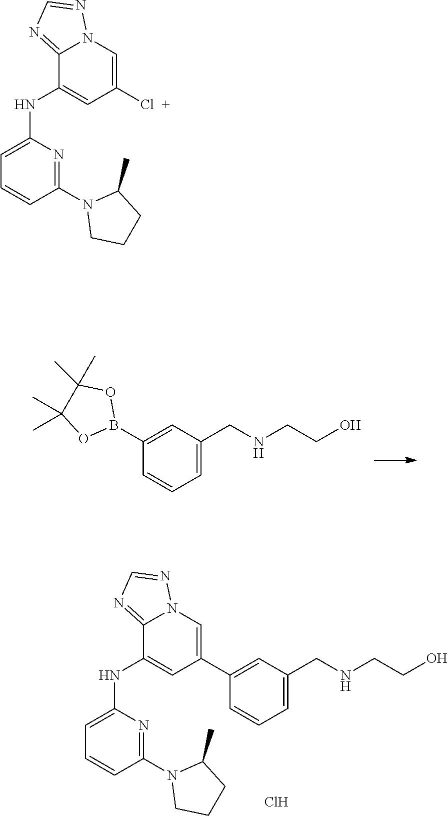

### Procedure:

A mixture of (S)-6-chloro-N-(6-(2-methylpyrrolidin-1-yl)pyridin-2-yl)-[1,2,4]triazolo[1,5-a]pyridin-8-amine (100 mg, 0.3 mmol), 2-(3-(4,4,5,5-tetramethyl-1,3,2-dioxaborolan-2-yl)benzylamino)ethanol (100 mg, 0.36 mmol), Pd₂(dba)₃(30 mg, 0.05 mmol), X-Phos (30 mg, 0.06 mmol) and Cs₂CO₃ (196 mg, 0.6 mmol) in dioxane/H₂O (30 mL/5 mL) was stirred at reflux for 18 h under N₂ atmosphere. The solvent was removed under reduced pressure and the residue purified by preparative-HPLC (Gemini 5u C18 150×21.2 mm; inject volume: 3 ml/inj, flow rate: 20 mL/min; wavelength: 214 nm and 254 nm; gradient conditions: 22% acetonitrile/78% water (0.1% TFA, v/v) initially, proceed to 27% acetonitrile/73% water (0.1% TFA, v/v) in a linear fashion after 9 min.). The solution of the purified compound was adjusted to pH=2 with 1N HCl and then concentrated to give (S)-2-(3-(8-(6-(2-methylpyrrolidin-1-yl)pyridin-2-ylamino)-[1,2,4]triazolo[1,5-a]pyridin-6-yl)benzylamino)ethanol hydrochloride (29 mg, 20%) as a yellow solid. ¹H NMR (300 MHz, DMSO): δ 9.35-9.30 (m, 3H, exchangeable by D₂O), 9.03 (s, 1H), 8.37 (s, 1H), 8.58 (s, 1H), 8.04 (s, 1H), 7.82 (d, 1H, J=7.2 Hz), 7.64-7.57 (m, 2H), 7.49-7.42 (m, 1H), 6.64 (d, 1H, J=7.8 Hz, exchangeable by D₂O), 6.06 (d, 1H, J=8.1 Hz, exchangeable by D₂O), 4.26 (brs, 3H), 3.74-3.71 (m, 2H), 3.58 (brs, 1H), 3.43-3.38 (m, 1H), 3.02 (brs, 2H), 2.09-1.98 (m, 3H), 1.69-1.67 (m, 1H), 1.09 (d, 3H, J=6.0 Hz). LC/MS: 444 [M+H]⁺. HPLC: 100% at 214 nm, 100% at 254 nm, t_{R}=4.53 min.

### Example 23

### (S)-3-(8-(6-(2-Methylpyrrolidin-1-yl)pyridin-2-ylamino)-[1,2,4]triazolo[1,5-a]pyridin-6-yl)benzamide

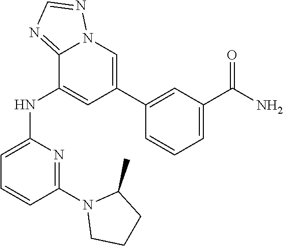

### Step 1

### (S)-3-(8-(6-(2-Methylpyrrolidin-1-yl)pyridin-2-ylamino)[1,2,4]triazolo[1,5-a]pyridin-6-yl)benzamide

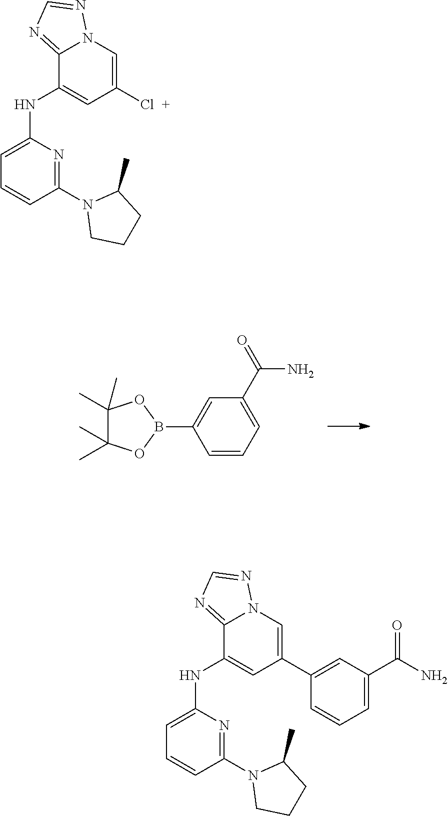

### Procedure:

A mixture of (S)-6-chloro-N-(6-(2-methylpyrrolidin-1-yl)pyridin-2-yl)-[1,2,4]triazolo[1,5-a]pyridin-8-amine (100 mg, 0.3 mmol), 3-(4,4,5,5-tetramethyl-1,3,2-dioxaborolan-2-yl)benzamide (89 mg, 0.36 mmol), Pd₂(dba)₃ (30 mg, 0.05 mmol), X-Phos (30 mg, 0.06 mmol) and Cs₂CO₃ (196 mg, 0.6 mmol) in dioxane/H₂O (30 mL/5 mL) were stirred at reflux for 18 h under N₂ atmosphere. The solvent was removed under reduce pressure and then the residue purified by preparative-HPLC (Gemini 5u C18 150×21.2 mm; inject volume: 3 ml/inj, flow rate: 20 mL/min; wavelength: 214 nm and 254 nm; gradient conditions: 27% acetonitrile/73% water (0.1% TFA, v/v) initially, proceed to 42% acetonitrile/58% water (0.1% TFA, v/v) in a linear fashion after 9 min.) to give (S)-3-(8-(6-(2-methylpyrrolidin-1-yl)pyridin-2-ylamino)-[1,2,4]triazolo[1,5-a]pyridin-6-yl)benzamide 2,2,2-trifluoroacetate (10 mg, 8%) as a yellow solid. ¹H NMR (300 MHz, DMSO): δ 9.21 (s, 1H), 9.12 (s, 1H), 8.82 (s, 1H), 8.55 (s, 1H), 8.28 (s, 1H), 8.13 (s, 1H), 7.95-7.91 (m, 2H), 7.63-7.38 (m, 3H), 6.63 (d, 1H, J=7.8 Hz), 5.99 (d, 1H, J=8.4 Hz), 4.19 (brs, 1H), 3.53 (brs, 1H), 3.37 (brs, 1H), 2.04-1.94 (m, 3H), 1.64 (brs, 1H), 1.04 (d, 3H, J=6.0 Hz). LC/MS: 414 [M+H]⁺. HPLC: 97.01% at 214 nm, 98.78% at 254 nm, t_{R}=5.20 min.

### Example 24

### (S)—N-(6-(2-Methylpyrrolidin-1-yl)pyridin-2-yl)-6-(3-((piperidin-4-ylamino)methyl)phenyl)-[1,2,4]-triazolo[1,5-a]pyridin-8-amine 2,2,2-trifluoroacetate

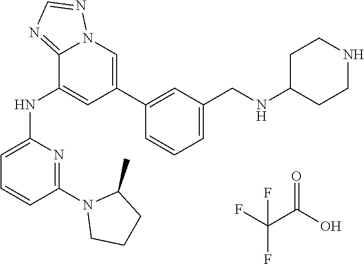

### Step 1

### (S)-tert-Butyl 4-(3-(8-(6-(2-methylpyrrolidin-1-yl)pyridin-2-ylamino)-[1,2,4]triazolo[1,5-a]pyridin-6-yl)benzylamino)piperidine-1-carboxylate

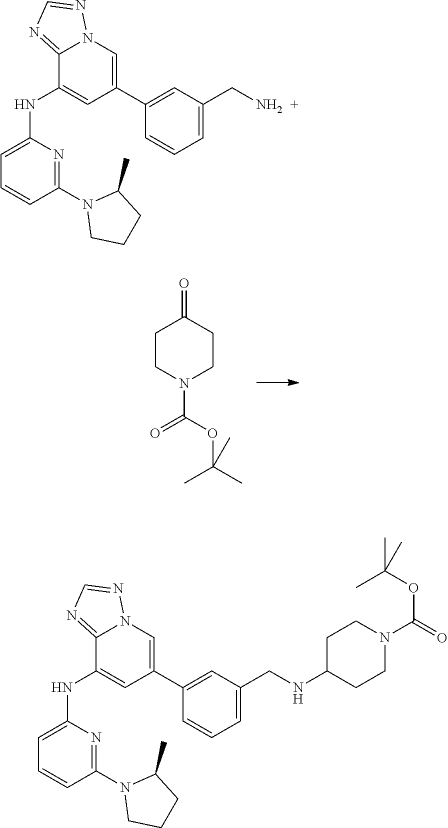

### Procedure:

A mixture of (S)-6-(3-(aminomethyl)phenyl)-N-(6-(2-methylpyrrolidin-1-yl)pyridin-2-yl)-[1,2,4]triazolo[1,5-a]pyridin-8-amine (50 mg, 0.125 mmol), tert-butyl 4-oxopiperidine-1-carboxylate (50 mg, 0.25 mmol), sodium triacetoxyborohydride (80 mg, 0.375 mmol) and HOAc (0.05 mL) in DCM (30 mL) were stirred at room temperature overnight. The solvent was removed under reduced pressure and the residue was filtered through a plug of silica gel. The cake was washed with EtOAc and the combined filtrates concentrated to give crude (S)-tert-butyl 4-(3-(8-(6-(2-methylpyrrolidin-1-yl)pyridin-2-ylamino)-[1,2,4]triazolo[1,5-a]pyridin-6-yl)benzylamino)piperidine-1-carboxylate (43 mg, 59%) as an oil. This was used directly without further purification. LC/MS: 583 [M+H]⁺.

### Step 2

### (S)—N-(6-(2-Methylpyrrolidin-1-yl)pyridin-2-yl)-6-(3-((piperidin-4-ylamino)methyl)phenyl)-[1,2,4]-triazolo[1,5-a]pyridin-8-amine 2,2,2-trifluoroacetate

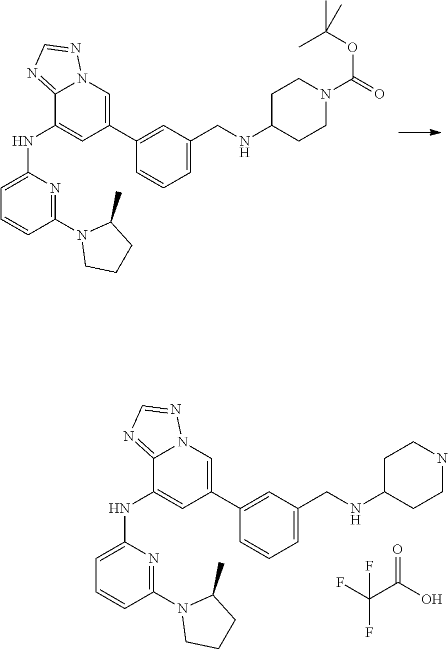

### Procedure:

The solution of (S)-tert-butyl 4-(3-(8-(6-(2-methylpyrrolidin-1-yl)pyridin-2-ylamino)-[1,2,4]triazolo[1,5-a]pyridin-6-yl)benzylamino)piperidine-1-carboxylate (43 mg, 0.074 mmol) in TFA/DCM (20 mL/20 mL) was stirred at room temperature for 1 hour. The solvent was removed under reduced pressure and then the residue purified by preparative-HPLC (Gemini 5u C18 150×21.2 mm; inject volume: 3 ml/inj, flow rate: 20 mL/min; wavelength: 214 nm and 254 nm; gradient conditions: 10% acetonitrile/90% water (0.1% TFA, v/v) initially, proceed to 50% acetonitrile/50% water (0.1% TFA, v/v) in a linear fashion after 9 min.) to give (S)—N-(6-(2-methylpyrrolidin-1-yl)pyridin-2-yl)-6-(3-((piperidin-4-ylamino)methyl)phenyl)-[1,2,4]triazolo[1,5-a]pyridin-8-amine 2,2,2-trifluoroacetate (11 mg, 25%) as a yellow solid. ¹H NMR (300 MHz, CD₃OD): δ 8.85 (s, 1H), 8.56 (s, 1H), 8.27 (s, 1H), 7.94-7.86 (m, 2H), 7.72-7.66 (m, 3H), 6.65 (d, 1H, J=8.1 Hz, partly exchangeable by CD₃OD), 6.36 (d, 1H, J=8.7 Hz, partly exchangeable by CD₃OD), 4.44 (s, 2H), 4.38-4.34 (m, 1H), 3.81-3.56 (m, 5H), 3.21-3.13 (m, 2H), 2.53-2.48 (m, 2H), 2.30-2.22 (m, 3H), 2.10-1.89 (m, 3H), 1.31 (d, 3H, J=6.6 Hz). LC/MS:, 483 [M+H]⁺. HPLC: 98.91% at 214 nm, 98.72% at 254 nm, t_{R}=4.16 min.

### Example 25

### 3-(8-(6-((3aR,6aS)-Hexahydrocyclopenta[c]pyrrol-2(1H)-yl)pyridin-2-ylamino)-[1,2,4]triazolo[1,5-a]pyridin-6-yl)benzamide

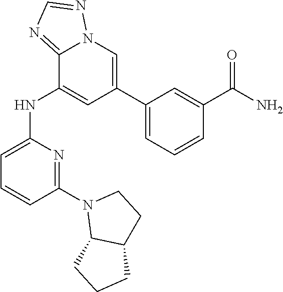

### Step 1

### (3R,6aR,9aS)-3-Phenylhexahydrocyclopenta[2,3]pyrrolo[2,1-b]oxazol-5(6H)-one

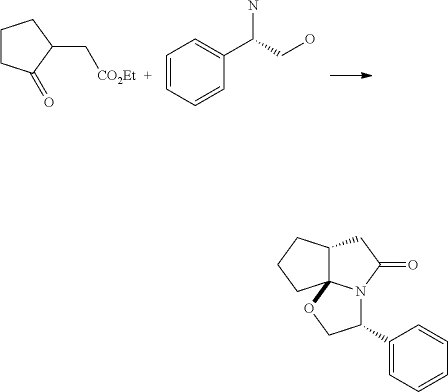

### Procedure:

A mixture of ethyl 2-(2-oxocyclopentyl)acetate (5 g, 29.3 mmol) and (S)-2-amino-2-phenylethanol (6.04 g, 44.0 mmol) was refluxed in 100 mL of toluene in a flask fitted with a Dean-stark trap. After 16 h, the reaction was cooled and concentrated at reduced pressure to give an orange solid. This was filtered through a plug of silica gel and the cake washed with petroleum ether/ethyl acetate (3:1, v/v). The combined filtrates were concentrated to give crude (3R,6aR,9aS)-3-phenylhexahydrocyclopenta[2,3]pyrrolo[2,1-b]oxazol-5(6H)-one (5.2 g, 72.7%) as a pale yellow solid. This was used directly without further purification. LC/MS: 244.1 [M+H]⁺.

### Step 2

### (R)-2-((3aR,6aR)-Hexahydrocyclopenta[b]pyrrol-1(2H)-yl)-2-phenylethanol

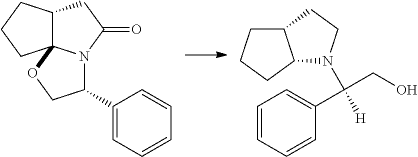

### Procedure:

A solution of (3R,6aR,9aS)-3-phenylhexahydrocyclopenta[2,3]pyrrolo[2,1-b]oxazol-5(6H)-one (5.2 g, 21.4 mmol) in 50 mL of anhydrous THF was cooled to −78° C. under nitrogen and treated drop wise with a 1M solution of BH₃ in THF (64 mL, 64 mmol). Stirring was continued at −78° C. for one hour, at which point the cooling bath was removed and the reaction was stirred at reflux for 2 h. Then the solution was stirred at room temperature for 16 h. The reaction was then placed in an ice bath and treated dropwise with 1N HCl (64 mL), then refluxed for 1.5 h, cooled and poured into 200 mL of brine. The mixture was concentrated at reduced pressure and the resulting aqueous layer was adjusted to pH=10 with 5N NaOH. The aqueous basic layer was extracted with dichloromethane (150 mL), and the organic phase was washed with brine (20 mL), dried over anhydrous sodium sulfate. The solvent was evaporated and the residue was filtered through a plug of silica gel and the cake washed with petroleum ether/ethyl acetate (3:5, v/v). The filtrates were combined and concentrated to give crude (R)-2-((3aR,6aR)-hexahydrocyclopenta[b]pyrrol-1(2H)-yl)-2-phenylethanol (4.5 g, 92%) as an oil. This was used directly without further purification. LC/MS: 232.2 [M+H]⁺.

### Step 3

### (3aR,6aR)-Octahydrocyclopenta[b]pyrrole hydrochloride

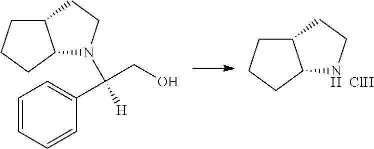

### Procedure:

To a stirred solution of (R)-2-((3aR,6aR)-hexahydrocyclopenta[b]pyrrol-1(2H)-yl)-2-phenylethanol (0.4 g, 1.73 mmol) in 35 mL of methanol was added 20% Pd(OH)₂/C (0.1 g) at room temperature under nitrogen. Then the reaction was evacuated with H₂ three times. After that, the reaction mixture was stirred at room temperature under H₂ for 16 h. The solution was treated with saturated HCl in dioxane to pH=1. The mixture was filtered and the filtrate was evaporated at 40° C. at reduced pressure, then the residue was triturated with 20 mL of ether to give (3aR,6aR)-octahydrocyclopenta[b]pyrrole hydrochloride (0.187 g, 73.3%) as a white solid. ¹H NMR (300 MHz, DMSO): δ 3.93-3.89 (m, 1H), 3.12-3.01 (m, 2H), 2.74-2.67 (m, 1H), 2.01-1.97 (m, 1H), 1.81-1.37 (m, 8H). LC/MS: 112.3 [M+H]⁺.

### Step 4

### (3aS,6aS)-1-(6-Nitropyridin-2-yl)-octahydrocyclopenta[b]pyrrole

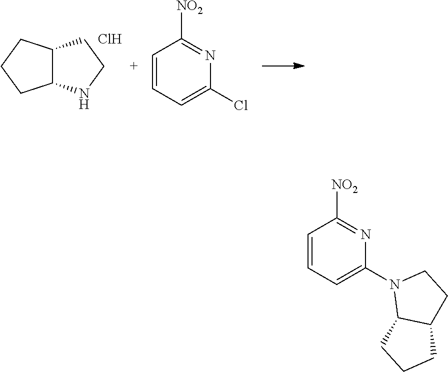

### Procedure:

To a stirred solution of 2-chloro-6-nitropyridine (0.266 g, 1.67 mmol) and (3aR,6aR)-octahydrocyclopenta[b]pyrrole hydrochloride (0.225 g, 1.52 mmol) in DMSO (7 mL) was added NaHCO₃ (0.211 g, 2.51 mmol) in one portion at room temperature. Then the reaction mixture was stirred at 80° C. for 16 h. The mixture was poured into water (20 mL) and extracted with ethyl acetate (90 mL). The organic phase was washed with water (15 mL), brine (10 mL) and dried over anhydrous sodium sulfate. The drying agent was removed by filtration and the filtrate was evaporated at 40° C. at reduced pressure and the residue was purified by column chromatography (silica gel, 200-300 mesh, ethyl acetate/petroleum ether 1:40, v/v) to give (3aS,6aS)-1-(6-nitropyridin-2-yl)-octahydrocyclopenta[b]pyrrole (0.19 g, 53.4%) as a yellow solid. ¹H NMR (300 MHz, CD₃Cl): δ 7.61 (t, 1H, J=7.9 Hz), 7.36 (d, 1H, J=7.5 Hz), 6.68 (d, 1H, J=8.4 Hz), 4.25-4.19 (m, 1H), 3.71-3.54 (m, 2H), 2.85-2.81 (m, 1H), 2.12-1.49 (m, 8H). LC/MS: 234.1 [M+H]⁺.

### Step 5

### 6-((3aS,6aS)-Hexahydrocyclopenta[b]pyrrol-1(2H)-yl)pyridin-2-amine

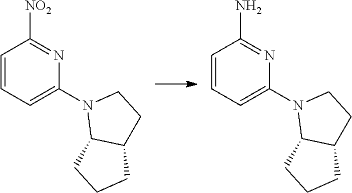

### Procedure:

To a stirred solution of (3aS,6aS)-1-(6-nitropyridin-2-yl)-octahydrocyclopenta[b]pyrrole (0.19 g, 0.81 mmol) in water (5 mL) and dioxane (10 mL) was added zinc dust (0.65 g, 10 mmol) in one portion at room temperature. Then concentrated HCl (1 mL) was added drop wise to the mixture over one hour. After the addition, the solution was treated with saturated NaHCO₃ solution to pH=7, then filtered. The filtrate was evaporated at 40° C. at reduced pressure. The residue was dissolved in ethyl acetate (20 mL), washed with water (5 mL), brine (5 mL) and dried over anhydrous sodium sulfate. The drying agent was removed by filtration and the filtrate was evaporated at 40° C. at reduced pressure to give a crude product. This was filtered through a plug of silica gel and the cake washed with ethyl acetate/petroleum ether (1:1, v/v). The combined filtrates were concentrated to give crude 6-((3aS,6aS)-hexahydrocyclopenta[b]pyrrol-1(2H)-yl)pyridin-2-amine (0.166 g, 100%) as a red oil. This was used directly without further purification. LC/MS: 204.2 [M+H]⁺.

### Step 6

### 3-(8-Bromo-[1,2,4]-triazolo[1,5-a]pyridin-6-yl)benzamide

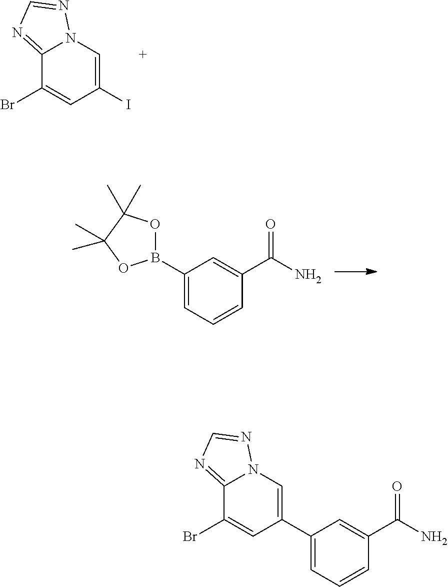

### Procedure:

To a stirred solution of 8-bromo-6-iodo-[1,2,4]triazolo[1,5-a]pyridine (0.391 g, 1.21 mmol), 3-(4,4,5,5-tetramethyl-1,3,2-dioxaborolan-2-yl)benzamide (0.10 g, 0.60 mmol) and Na₂CO₃ (0.33 g, 3.11 mmol) in water (3 mL) and dioxane (70 mL) was added Pd(PPh₃)₄ (0.095 g, 0.082 mmol) in one portion at room temperature under nitrogen. Then the reaction mixture was degassed by bubbling nitrogen for 10 minutes and then stirred at 97° C. for 16 h under nitrogen. The mixture was evaporated at 40° C. at reduced pressure and the residue was filtered through a plug of silica gel and the cake washed with ethyl acetate/methanol (10:1, v/v). The filtrates were combined and concentrated to give crude 3-(8-bromo-[1,2,4]triazolo[1,5-a]pyridin-6-yl)benzamide (0.157 g, 81.7%) as a pale yellow solid. This was used directly without further purification. LC/MS: 316.9 [M+H]⁺.

### Step 7

### 3-(8-(6-((3aS,6aS)-Hexahydrocyclopenta[b]pyrrol-1(2H)-yl)pyridin-2-ylamino)-[1,2,4]-triazolo[1,5-a]pyridin-6-yl)benzamide

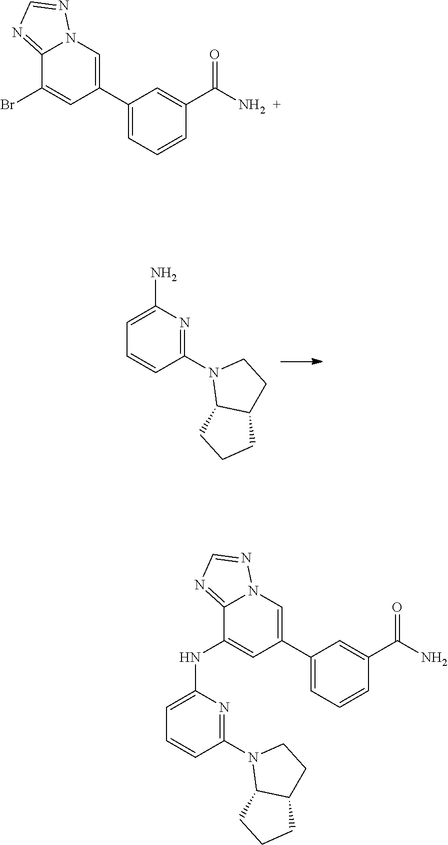

### Procedure:

To a stirred solution of 3-(8-bromo-[1,2,4]triazolo[1,5-a]pyridin-6-yl)benzamide (0.157 g, 0.495 mmol), 6-((3aS,6aS)-hexahydrocyclopenta[b]pyrrol-1(2H)-yl)pyridin-2-amine (0.10 g, 0.195 mmol), BINAP (0.130 g, 0.209 mmol) and Cs₂CO₃ (0.5 g, 1.5 mmol) in dioxane (40 mL) was added Pd₂(dba)₃ (0.05 g, 0.054 mmol) in one portion under nitrogen at room temperature. Then the mixture was degassed by bubbling nitrogen for 10 minutes and then stirred at 96° C. under nitrogen for 16 h. The solvent was evaporated at 40° C. at reduced pressure and the residue was purified by column chromatography (silica gel, 200-300 mesh, ethyl acetate/methanol 10:1, v/v) to give 3-(8-(6-((3aS,6aS)-hexahydrocyclopenta[b]pyrrol-1(2H)-yl)pyridin-2-ylamino)-[1,2,4]triazolo[1,5-a]pyridin-6-yl)benzamide containing additional unidentified impurities. This was purified further by preparative-HPLC (Gemini 5u C18 150×21.2 mm; inject volume: 3 ml/inj, flow rate: 20 mL/min; wavelength: 214 nm and 254 nm; gradient conditions: 53% acetonitrile/47% water (0.1% TFA, v/v) initially, proceed to 61% acetonitrile/39% water (0.1% TFA, v/v) in a linear fashion after 9 min.) to give 3-(8-(6-((3aS,6aS)-hexahydrocyclopenta[b]pyrrol-1(2H)-yl)pyridin-2-ylamino)-[1,2,4]triazolo[1,5-a]pyridin-6-yl)benzamide 222-trifluoroacetate (10 mg, 4.6%) as a green solid. ¹H NMR (300 MHz, DMSO): δ 9.27 (s, 1H), 9.18 (s, 1H), 8.83 (s, 1H), 8.55 (s, 1H), 8.28 (s, 1H), 8.14 (s, 1H), 7.92 (s, 1H), 7.60 (t, 1H, J=7.7 Hz), 7.52 (brs, 1H), 7.39 (t, 1H, J=8.0 Hz), 6.66 (d, 1H, J=7.8 Hz), 6.01 (d, 1H, J=8.1 Hz), 4.24 (brs, 1H), 2.75-2.68 (m, 2H), 2.05-1.98 (m, 2H), 1.66-1.31 (m, 7H). LC/MS: 440 [M+H]⁺. HPLC: 98.70% at 214 nm, 98.90% at 254 nm, t_{R}=5.47 min.

### Biological Examples

### SYK Assay Information

### Determination of IC₅₀ of Spleen Tyrosine Kinase (SYK) Inhibition

SYK kinase assay is a standard kinase assay adapted to a 96 well plate format. This assay is performed in 96-well format for IC₅₀ determination with 8 samples which represented 10 half log dilutions and a 40 μL reaction volume. The assay measures the incorporation of radiolabeled ³³P γATP into an N-terminally biotinylated peptide substrate, derived from naturally occurring phosphoacceptor consensus sequence (Biotin-11aa DY*E). Phosphorylated products were detected upon termination of reactions with EDTA and the addition of Streptavidin coated beads. Representative results are in Table II above.

Assay plates: 96-well MultiScreen 0.65 um filter plates (Millipore Cat. No.: MADVNOB10)

Streptavidin coated beads: Streptavidin Sepharose™, suspension 5.0 mL, in 50 mM EDTA/PBS diluted (1:100), (Amersham, Cat. No.: 17-5113-01)
Compounds: 10 mM in 100% dimethylsulfoxide (DMSO), final conc.: compound 0.003-100 uM in 10% DMSO
Enzyme: Recombinant human full length SYK protein (Invitrogen Cat. No.: PV4089) dephosphotylated by protein tyrosine phosphatase PTP1B, working solution 8.89 nM, final conc.:0.004 μM.
Peptide 1: biotinylated peptide is derived from a naturally occurring phosphor-acceptor con-sensus sequence (Biotin-EPEGDYEEVLE), special order from QCB, stock solution 20 mM, final conc.: 10 μM.
ATP: Adenosine-5′-triphosphate 20 mM, (ROCHE Cat. No.: 93202720), final concentration: 20 μM
Buffer: HEPES: 2-Hydroxyethyl piperazine-2-ethanesulfonic acid (Sigma, Cat. No.: H-3375) final concentration: 50 mM HEPES pH7.5
BSA: Bovine Serum Albumin Fraction V, fatty acid free (Roche Diagnostics GmbH, Cat. No. 9100221) diluted to a final concentration of 0.1%
EDTA: EDTA stock solution 500 mM, (GIBCO, Cat. No.: 15575-038) final concentration: 0.1 mM
DTT: 1,4-Dithiothreitol (Roche Diagnostics GmbH, Cat. No.: 197777), final conc.: 1 mM MgCl₂×6H₂O: MERCK, Cat. No.: 105833.1000, final concentration: 10 mM
Assay Dilution Buffer (ADB): 50 mM HEPES, 0.1 mM EGTA, 0.1 mM Na Vanadate, 0.1 mM β-glycerophosphate, 10 mM MgCl₂, 1 mM DTT, 0,1% BSA, pH 7.5
Bead wash buffer: 10 g/L PBS (Phosphate buffered saline) with 2M NaCl+ 1% phosphoric acid.

### Experimental Method

In 20 μL volume, 18 μL of recombinant human full length SYK [8.89 nM] was mixed with 2 μL of 10× concentrations of the test compounds, [usually 100 μM-0.003 μM] in [10%] DMSO and the mixture was incubated for 15 min at RT.

The kinase reaction was initiated by the addition of 20 μL 2× substrate cocktail containing the Biotin-peptide substrate [20 μM], ATP [40 μM] and ³³PγATP [2μCi/rxn]. After incubation at RT for 30 min, the reaction was terminated by the transfer of 25 μL of the reaction sample to a 96 well 0.65μm Millipore MADVNOB membrane/plate containing 100 μL 5 mM EDTA and 10% Streptavidine coated beads in PBS.

The unbound radionucleotides were washed under vacuum with 3×250 μL 2M NaCl; 2×250 μL 2M NaCl+1% phosphoric acid and 1×250 μL H₂O. After the last wash, membrane/plates were transferred to an adaptor plate, heat dried for 1 hour min at 60° C., and 60 μL scintillation cocktail was added to each well and the amount of radioactivity was counted in a top counter.

The percent inhibition was calculated based on the uninhibited enzyme rate:

% Inhibition=(1-((Test−Positive Control)/(Negative Control−Positive Control)))*100

The IC₅₀ was calculated using a non-linear curve fit with XLfit software (ID Business Solution Ltd., Guilford, Surrey, UK).

| | IC₅₀ h-syk-gst-sf9-c (inactive- |
|---|---|
| Compound | dephosphorylated)/M |
| I-1 | 0.206 |
| I-2 | 0.06542 |
| I-3 | 0.0026 |
| I-4 | 0.3972 |
| I-5 | 0.01353 |
| I-6 | 2.8077 |
| I-7 | 0.06465 |
| I-8 | 0.04882 |
| I-9 | 4.86775 |
| I-10 | 0.0149 |
| I-11 | 0.00665 |
| I-12 | 0.51345 |
| I-13 | 2.63047 |
| I-14 | 1.11725 |
| I-15 | 0.0635 |
| I-16 | 2.42635 |
| I-17 | 0.24875 |
| I-18 | 0.10965 |
| I-19 | 0.1085 |
| I-20 | 0.05945 |
| I-21 | 0.13005 |
| I-22 | 0.06795 |
| I-23 | 0.0307 |
| I-24 | 0.18975 |
| I-25 | 0.28425 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound of Formula I 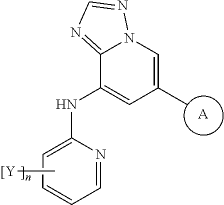
wherein:
A is phenyl or heterocycloalkyl, optionally substituted with one or more A′;
each A′ is independently C(═O)NHR, C(═O)R, C(═O)OR, OR, NHC(═O)R, CH₂NHR, lower alkyl, hydroxy lower alkyl, or hydroxy lower alkyl amino;
each R is independently H, or R′;
each R′ is independently lower alkyl, heterocycloalkyl, phenyl, heteroaryl, heteroaryl lower alkyl, or bicyclic heteroaryl, optionally substituted with one or more R″;
each R″ is independently hydroxy, lower alkyl amido, carboxy, oxo, lower alkoxy, lower alkyl amino, or lower dialkyl amino;
each Y is independently lower alkyl, lower alkoxy, lower haloalkyl, lower hydroxyalkyl, or heterocycloalkyl, optionally substituted with lower alkyl; and
n is 0, 1, or 2;
or a pharmaceutically acceptable salt thereof.

2. The compound of claim 1, wherein A is phenyl.

3. The compound of claim 2, wherein A′ is C(═O)NHR.

4. The compound of claim 3, wherein R is H.

5. The compound of claim 3, wherein R is phenyl, optionally substituted with one or more R″.

6. The compound of claim 3, wherein R is phenyl, optionally substituted with carboxy.

7. The compound of claim 3, wherein R is phenyl, optionally substituted with carboxy and methoxy.

8. The compound of claim 3, wherein R is heteroaryl, optionally substituted with one or more R″.

9. The compound of claim 3, wherein R is bicyclic heteroaryl, optionally substituted with one or more R″.

10. The compound of claim 1, wherein n is 2 and both Y are methoxy.

11. The compound of claim 1, wherein n is 1 and Y is methylpyrrolidinyl.

12. A compound selected from the group consisting of:
[1,4]Diazepan-1-yl-{3-[8-(5,6-dimethoxy-pyridin-2-ylamino)-[1,2,4]triazolo[1,5-a]pyridin-6-yl]-phenyl}-methanone;
3-[8-(5,6-Dimethoxy-pyridin-2-ylamino)-[1,2,4]triazolo[1,5-a]pyridin-6-yl]-N-(4-N-methyl)-benzamide
4-{3-[8-(5,6-Dimethoxy-pyridin-2-ylamino)-[1,2,4]triazolo[1,5-a]pyridin-6-yl]-benzoylamino}-benzoic acid;
4-[8-(5,6-Dimethoxy-pyridin-2-ylamino)-[1,2,4]triazolo[1,5-a]pyridin-6-yl]-N-(2-pyridin-4-yl-ethyl)-benzamide;
4-{3-[8-(5,6-Dimethoxy-pyridin-2-ylamino)-[1,2,4]triazolo[1,5-a]pyridin-6-yl]-benzoylamino}-2-methoxy-benzoic acid;
3-[8-(5,6-Dimethoxy-pyridin-2-ylamino)-[1,2,4]triazolo[1,5-a]pyridin-6-yl]-N-(2-oxo-2,3-dihydro-1H-indol-5-yl)-benzamide;
3-[8-(5,6-Dimethoxy-pyridin-2-ylamino)-[1,2,4]triazolo[1,5-a]pyridin-6-yl]-N-(1H-indazol-5-yl)-benzamide;
4-(3-{8-[6-(2-Methyl-pyrrolidin-1-yl)-pyridin-2-ylamino]-[1,2,4]triazolo[1,5-a]pyridin-6-yl}-benzoylamino)-benzoic acid;
4-{8-[6-(2-Methyl-pyrrolidin-1-yl)-pyridin-2-ylamino]-[1,2,4]triazolo[1,5-a]pyridin-6-yl}-benzoic acid methyl ester;
N-{1-[8-(5,6-Dimethoxy-pyridin-2-ylamino)-[1,2,4]triazolo[1,5-a]pyridin-6-yl]-piperidin-3-yl}-terephthalamic acid;
2-Methoxy-4-(3-{8-[6-(2-methyl-pyrrolidin-1-yl)-pyridin-2-ylamino]-[1,2,4]triazolo[1,5-a]pyridin-6-yl}-benzoylamino)-benzoic acid;
4-{8-[6-(2-Methyl-pyrrolidin-1-yl)-pyridin-2-ylamino]-[1,2,4]triazolo[1,5-a]pyridin-6-yl}-benzoic acid;
3-{8-[6-(2-Methyl-pyrrolidin-1-yl)-pyridin-2-ylamino]-[1,2,4]triazolo[1,5-a]pyridin-6-yl}-benzoic acid methyl ester;
3-{8-[6-(2-Methyl-pyrrolidin-1-yl)-pyridin-2-ylamino]-[1,2,4]triazolo[1,5-a]pyridin-6-yl}-benzoic acid;
4-({1-[8-(5,6-Dimethoxy-pyridin-2-ylamino)-[1,2,4]triazolo[1,5-a]pyridin-6-yl]-piperidine-3-carbonyl}-amino)-benzoic acid;
3-{8-[(3aS,6aS)-6-(Hexahydro-cyclopenta[b]pyrrol-1-yl)-pyridin-2-ylamino]-[1,2,4]triazolo[1,5-a]pyridin-6-yl}-benzamide;
4-[8-(5,6-Dimethoxy-pyridin-2-ylamino)-[1,2,4]triazolo[1,5-a]pyridin-6-yl]-N-(2-dimethylamino-ethyl)-benzamide;
4-[8-(5,6-Dimethoxy-pyridin-2-ylamino)-[1,2,4]triazolo[1,5-a]pyridin-6-yl]-benzamide;
4-{8-[6-((S)-2-Methyl-pyrrolidin-1-yl)-pyridin-2-ylamino]-[1,2,4]triazolo[1,5-a]pyridin-6-yl}-benzamide;
{3-[8-(5,6-Dimethoxy-pyridin-2-ylamino)-[1,2,4]triazolo[1,5-a]pyridin-6-yl]-phenyl}-methanol;
N-(2-Dimethylamino-ethyl)-4-{8-[6-((S)-2-methyl-pyrrolidin-1-yl)-pyridin-2-ylamino]-[1,2,4]triazolo[1,5-a]pyridin-6-yl}-benzamide; compound with trifluoro-acetic acid;
(3-{8-[6-((S)-2-Methyl-pyrrolidin-1-yl)-pyridin-2-ylamino]-[1,2,4]triazolo[1,5-a]pyridin-6-yl}-phenyl)-methanol;
2-(3-{8-[6-((S)-2-Methyl-pyrrolidin-1-yl)-pyridin-2-ylamino]-[1,2,4]triazolo[1,5-a]pyridin-6-yl}-benzylamino)-ethanol;
3-{8-[6-((S)-2-Methyl-pyrrolidin-1-yl)-pyridin-2-ylamino]-[1,2,4]triazolo[1,5-a]pyridin-6-yl}-benzamide; and
[6-((S)-2-Methyl-pyrrolidin-1-yl)-pyridin-2-yl]-{6-[3-(piperidin-4-ylaminomethyl)-phenyl]-[1,2,4]triazolo[1,5-a]pyridin-8-yl}-amine.

13. A method for treating an inflammatory or autoimmune condition comprising administering to a patient in need thereof a therapeutically effective amount of the compound of claim 1.

14. The method of claim 13, further comprising administering an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, or an agent for treating immunodeficiency disorders.

15. A method for treating an inflammatory condition comprising administering to a patient in need thereof a therapeutically effective amount of claim 1.

16. A method for treating rheumatoid arthritis comprising administering to a patient in need thereof a therapeutically effective amount of the compound of claim 1.

17. A method for treating asthma comprising administering to a patient in need thereof a therapeutically effective amount of the compound of claim 1.

18. A method for treating an immune disorder including lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease, and Leukemia, comprising administering to a patient in need thereof a therapeutically effective amount of the compound of claim 1.

19. A method for treating an inflammatory condition comprising co-administering to a patient in need thereof a therapeutically effective amount of an anti-inflammatory compound in combination with the compound of claim 1.

20. A method for treating an immune disorder comprising co-administering to a patient in need thereof a therapeutically effective amount of an immunosuppressant compound in combination with the compound of claim 1.

21. A pharmaceutical composition comprising the compound of claim 1, admixed with at least one pharmaceutically acceptable carrier, excipient or diluent.

22. The pharmaceutical composition of claim 21, further comprising an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, and an agent for treating immunodeficiency disorders.
